(19) United States
(12) Patent Application Publication
(10) Pub. No.: US 2024/0391969 A1
(43) Pub. Date: Nov. 28, 2024

(54) METHODS AND COMPOSITIONS TO SPREAD PROTEIN CARGOES ACROSS MULTI-NUCLEATED CELLS

(71) Applicant: University of Florida Research Foundation, Incorporated, Gainesville, FL (US)

(72) Inventors: Eric Tzy-Shi Wang, Gainesville, FL (US); Keril K. Poukalov, Gainesville, FL (US)

(73) Assignee: University of Florida Research Foundation, Incorporated, Gainesville, FL (US)

(21) Appl. No.: 17/927,337

(22) PCT Filed: May 21, 2021
(86) PCT No.: PCT/US2021/033680

§ 371 Date: Nov. 22, 2022

Related U.S. Application Data
(60) Provisional application No. 63/029,303, filed on May 22, 2020.

(51) Int. Cl. C07K 14/47; A61K 48/00; C12N 15/86

(52) U.S. Cl. CPC C07K 14/4707; A61K 48/0058; C12N 15/86; C07K 2319/09; C07K 2319/095; C12N 2750/14122; C12N 2750/14143

(57) ABSTRACT

Methods and compositions useful for delivering a protein to a plurality of nuclei in a cell. Fusion proteins comprising one or more signals useful for promoting delivery of the proteins to a plurality of nuclei in a multinucleate cell. Methods comprising administering isolated nucleic acids to a multinucleate cell, wherein the isolated nucleic acid comprises a sequence encoding a fusion protein, the fusion protein comprising, a protein of interest fused to at least the following migration signals: (a) at least one nuclear export signal (NFS); and (b) at least one nuclear localization signal (NLS) and/or at least one nucleolar localization signal (NoLS). Fusion proteins comprising: (a) a protein of interest; and (b) at least the following migration signals: (i) a nuclear export signal (NFS); (ii) a nuclear localization signal (NLS); and (iii) a nucleolar localization signal (NoLS). Methods of making and using the same. Methods of delivering recombinant genes and/or fusion proteins to multinucleate cells.

### CROSS-REFERENCE TO RELATED APPLICATIONS

This application is a national stage filing under 35 U.S.C. 371 of International Patent Application Serial No. PCT/US2021/033680, filed May 21, 2021, which claims the benefit under 35 U.S.C. § 119(e) of the filing date of U.S. Patent Application Ser. No. 63/029,303, filed May 22, 2020, the contents of which are hereby incorporated herein in its entirety by reference.

### STATEMENT REGARDING FEDERALLY SPONSORED RESEARCH OR DEVELOPMENT

This invention was made with government support under grant number DP5 O017865 and RO1 AG058636 awarded by the National Institutes of Health. The government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Aug. 13, 2023, is named U119670081US01-SUBSEQ-PRW and is 190, 610 bytes in size.

### BACKGROUND OF THE INVENTION

Gene therapy has become an intense focus of research over the past decade. The focus of gene therapies has been to effect a change in an organism such that the organism itself produces a beneficial product, refrains from making a deleterious product, or a combination thereof. However, for such therapies to be successful, they should target the appropriate cellular machinery, including targeting relevant nucleic acids, and accomplish such targeting with a great enough efficiency to effectuate the change without causing toxicity or producing deleterious off-target effects. Various techniques have been employed to achieve this targeting of cellular reprogramming, with mixed results. Examples of some techniques are the inclusion of nuclear localization signals (NLS) and nuclear export signals (NES). However, while these signals may facilitate nuclear entry, they can inhibit and/or prevent the effective and efficient spread of such gene therapies across multiple nuclei, which is problematic, for example, in multinucleate cells. New directions and therapies that could improve understanding and treating of multinucleate cells are desperately needed.

### SUMMARY OF THE INVENTION

In some aspects, the disclosure relates to methods and compositions for delivering recombinant genes and/or fusion proteins to multinucleate cells. In some embodiments, the method comprises administering an isolated nucleic acid to a multinucleate cell, wherein the isolated nucleic acid comprises a sequence encoding a fusion protein, the fusion protein comprising, a protein of interest fused to at least the following migration signals: (a) at least one nuclear export signal (NES); and (b) at least one nuclear localization signal (NLS) and/or at least one nucleolar localization signal (NoLS). In some embodiments, an isolated nucleic acid encodes an amino acid sequence comprising a sequence with at least 70% identity to SEQ ID NO: 13.

In some embodiments, fusion protein further comprises at least one additional migration signal, wherein the additional migration signal may be identical or distinct from the existing migration signals of the fusion protein. In some embodiments, the fusion protein further comprises at least two additional migration signals, wherein the additional migration signals may be identical or distinct from the existing migration of the fusion protein.

In some embodiments, the isolated nucleic acid encodes a fusion protein wherein at least one of the migration signals is positioned at the C-terminus of the protein of interest. In some embodiments, the isolated nucleic acid encodes a fusion protein wherein at least one of the migration signals is positioned at the N-terminus of the protein of interest. In some embodiments, a fusion protein comprises a sequence with at least 70% identity to SEQ ID NO: 13. In some embodiments, a fusion protein comprises a migration signal comprising a sequence with at least 70% identity to a sequence of any one of SEQ ID NO: 14-570. In some embodiments, a fusion protein comprises a migration signal comprising a sequence of any one of SEQ ID NO: 14-570.

In some embodiments, the protein of interest is a therapeutic protein. In some embodiments, the protein of interest is a nuclear protein. In some embodiments, the protein of interest is a transcriptional factor, transcriptional repressor, RNA binding protein. DNA modifying protein (e.g., enzyme), DNA editing protein (e.g., enzyme). Cas protein (e.g., Cas9. Cas13, etc . . . ). In some embodiments, the protein of interest is DUX4. In some embodiments the protein of interest may be used to treat Facioscapulohumeral dystrophy. In some embodiments, the protein of interest may carry RNA (e.g., mRNA, miRNA (microRNA), shRNA (short hairpin RNA, small hairpin RNA), gRNA (guide RNA)). In some embodiments, the protein of interest may carry RNA to multiple nuclei.

In some embodiments, the isolated nucleic acid is flanked by adeno-associated virus (AAV) inverted terminal repeats (ITRs).

In some embodiments, the multinucleate cell is a skeletal muscle cell.

In some embodiments, the nucleic acid is administered to a subject. In some embodiments, the subject is mammalian. In some embodiments, the subject is human.

In some aspects, the disclosure relates to a fusion protein comprising: (a) a protein of interest, and (b) at least the following migration signals: (i) a nuclear export signal (NES); (ii) a nuclear localization signal (NLS); and (iii) a nucleolar localization signal (NoLS). In some embodiments, the fusion protein further comprises at least one additional migration signal, wherein the additional migration signal may be identical or distinct from the existing migration signals of the fusion protein. In some embodiments, the fusion protein further comprises at least two additional migration signals, wherein the additional migration signals may be identical or distinct from the existing migration signals of the fusion protein.

In some embodiments, at least one of the migration signals is positioned at the C-terminus of the protein of interest. In some embodiments, at least one of the migration signals is positioned at the N-terminus of the protein of interest.

In some embodiments, at least one of the migration signals is linked to the protein of interest via a linker. In some embodiments, at least one of the migration signals is linked to at least one other migration signal via a linker.

In some embodiments, at least one of the migration signals comprises a sequence with at least 95% identity to SEQ ID NO: 1. In some embodiments, at least one of the migration signals comprises a sequence with at least 95% identity to SEQ ID NO: 3. In some embodiments, at least one of the migration signals comprises a sequence of SEQ ID NO: 1. In some embodiments, at least one of the migration signals comprises a sequence of SEQ ID NO: 3. In some embodiments, at least one of the migration signals comprises a sequence of SEQ ID NO: 1 and at least one of the migration signals comprises a sequence of SEQ ID NO: 3. In some embodiments, a migration signal comprises a sequence with at least 70% identity to a sequence of any one of SEQ ID NO: 14-570. In some embodiments, a migration signal comprises a sequence of any one of SEQ ID NO: 14-570.

In some embodiments, at least one of the migration signals is linked to at least one other migration signal via a linker.

In some aspects, the disclosure relates to an isolated nucleic acid comprising a nucleic acid sequence encoding at least one of the fusion proteins of the disclosure or of the methods of the disclosure.

In some embodiments, an isolated nucleic acid further comprises a promoter operably linked to a sequence encoding a fusion protein. In some embodiments, a promoter is a constitutive promoter, an inducible promoter, or a tissue specific promoter. In some embodiments, a promoter is a tissue specific promoter. In some embodiments, a tissue specific promoter is specific to skeletal muscle. In some embodiments, a tissue specific promoter is specific to liver tissue.

In some embodiments, an isolated nucleic acid further comprises at least one additional regulatory sequence.

In some aspects, the disclosure relates to a recombinant adeno-associated virus (rAAV), comprising: (a) an isolated nucleic acid as described herein flanked by adeno-associated virus (AAV) inverted terminal repeats (ITRs); and (b) an AAV capsid protein.

In some embodiments, AAV capsid protein exhibits a tropism for skeletal muscle. In some embodiments, an AAV capsid protein exhibits a tropism for liver tissue.

In some embodiments, an AAV capsid protein serotype is selected from: AAV1 and AAV6. In some embodiments, an AAV capsid protein serotype is AAV6. In some embodiments, an AAV capsid protein serotype is selected from: AAV7; AAV8; and AAV9.

In some aspects, the disclosure relates to a composition comprising any of the isolated nucleic acids of the disclosure, or any of the rAAVs of the disclosure, and a pharmaceutically acceptable excipient.

In some embodiments, a composition is formulated for intramuscular administration, intrathecal administration, intravenous administration, retrograde intravenous administration, intravascular administration, systemic administration, intra-arterial administration, intraportal administration, intratracheal administration, or direct injection.

In some aspects, the disclosure relates to a method of delivering a fusion protein to multinucleate cells, comprising, administering any of the fusion proteins of the disclosure, any of the isolated nucleic acids of the disclosure, any of the rAAVs of the disclosure, or any of the compositions of the disclosure to a subject.

In some embodiments, a protein of interest is a therapeutic protein in any of the fusion proteins of the disclosure, any of the isolated nucleic acids of the disclosure, any of the rAAVs of the disclosure, or any of the composition of the disclosure.

In some embodiments, a protein of interest is a transcriptional factor, transcriptional repressor, RNA binding protein, DNA modifying protein, DNA editing protein, Cas protein, DUX4, or variant thereof (e.g., dominant negative mutant), or an protein carrying an RNA in any of the fusion proteins of the disclosure, any of the isolated nucleic acids of the disclosure, any of the rAAVs of the disclosure, or any of the composition of the disclosure.

In some embodiments, a protein of interest is a nuclear protein in any of the fusion proteins of the disclosure, any of the isolated nucleic acids of the disclosure, any of the rAAVs of the disclosure, or any of the composition of the disclosure.

In some embodiments, a subject is mammalian. In some embodiments, a subject is human. In some embodiments, a subject has a disorder. In some embodiments, a disorder is Facioscapulohumeral dystrophy.

In some aspects, the disclosure relates to an isolated nucleic acid encoding at least one of the fusion proteins as disclosed herein.

In some embodiments, the isolated nucleic acid further comprises a promoter operably linked to the isolated nucleic acid encoding the fusion protein. In some embodiments, the promoter is a constitutive promoter, an inducible promoter, or a tissue specific promoter. In some embodiments, the promoter is a tissue specific promoter. In some embodiments, the tissue specific promoter is specific to skeletal muscle. In some embodiments, the tissue specific promoter is specific to liver tissue.

In some embodiments, the isolated nucleic acid further comprises at least one additional regulatory sequence.

In some aspects, the disclosure relates to a recombinant adeno-associated virus (rAAV), comprising: (a) at least one of the isolated nucleic acids as described herein flanked by adeno-associated virus (AAV) inverted terminal repeats (ITRs); and (b) an AAV capsid protein.

In some embodiments, the AAV capsid protein exhibits a tropism for skeletal muscle. In some embodiments, the AAV capsid protein serotype is selected from: AAV1. AAV6; AAV7; AAV8; and AAV9.

In some aspects, the disclosure relates to a composition comprising at least one of the isolated nucleic acids as described herein, or at least one of the rAAVs as disclosed herein, and a pharmaceutically acceptable excipient.

In some embodiments, the composition is formulated for intramuscular administration.

In some aspects, the disclosure relates to a method of delivering at least one of the fusion proteins as disclosed herein, to multinucleate cells. In some embodiments, the method comprises, administering at least one of the fusion proteins as disclosed herein, at least one of the isolated nucleic acids as disclosed herein, at least one of the rAAVs as disclosed herein, or at least one of the compositions as disclosed herein, to a subject (e.g., to one or more multinucleate cells in a subject).

In some embodiments, the protein of interest used in any of the fusion proteins as disclosed herein, any of the isolated nucleic acids as disclosed herein, any of the rAAVs as disclosed herein, or any of the compositions as disclosed herein, or as used in any of the methods as disclosed herein, may be a therapeutic protein.

In some embodiments, the protein of interest used in any of the fusion protein as disclosed herein, any of the isolated nucleic acids as disclosed herein, any of the rAAVs as disclosed herein, or any of the compositions as disclosed herein, or as used in any of the methods as disclosed herein, wherein the protein of interest is a nuclear protein.

In some embodiments, the protein of interest used in any of the fusion protein as disclosed herein, any of the isolated nucleic acids as disclosed herein, any of the rAAVs as disclosed herein, or any of the compositions as disclosed herein, or as used in any of the methods as disclosed herein, wherein the protein of interest is a transcriptional factor, transcriptional repressor. RNA binding protein. DNA modifying protein (e.g., enzyme). DNA editing protein (e.g., enzyme), Cas protein (e.g., Cas9, Cas13, etc. . . . ). In some embodiments, the protein of interest is DUX4 or variant thereof e.g., dominant negative form of DUX4). In some embodiments the protein of interest may be used to treat Facioscapulohumeral dystrophy. In some embodiments, the protein of interest may carry RNA (e.g., mRNA, miRNA (microRNA), shRNA (short hairpin RNA, small hairpin RNA), gRNA (guide RNA)). In some embodiments, the protein of interest may carry RNA to multiple nuclei.

In some embodiments, the subject in any of the methods as described herein is mammalian. In some embodiments, the subject is human.

These and other aspects and embodiments will be described in greater detail herein. The description of some exemplary embodiments of the disclosure are provided for illustration purposes only and not meant to be limiting. Additional compositions and methods are also embraced by this disclosure.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description. Drawings, Examples, and Claims.

### BRIEF DESCRIPTION OF THE DRAWINGS

Further aspects of the disclosure will be readily appreciated upon review of the Detailed Description of its various aspects and embodiments, described below, when taken in conjunction with the accompanying Drawings.

FIGS. 1A-1F show expression of GFP in C2C12 murine myoblasts transfected with NCT_GFP_017 plasmid. FIG. 1A. FIG. 1C, and FIG. 1E: GFP Signal of myoblasts post serum withdrawal to form myotubes. FIGS. 1B, FIG. 1D, and FIG. 1F: Brightfield Imaging of myoblasts post serum withdrawal to form myotubes.

FIGS. 2A-2D show expression of GFP in C2C12 murine myoblasts transfected with NCT_GFP_018 plasmid. FIG. 2A and FIG. 2C: GFP Signal of myoblasts post scrum withdrawal to form myotubes. FIG. 2B and FIG. 2D: Brightfield Imaging of myoblasts post serum withdrawal to form myotubes.

FIGS. 3A-3F show expression of GFP in C2C12 murine myoblasts transfected with NCT_GFP_021 plasmid. FIG. 3A, FIG. 3C, and FIG. 3E: GFP Signal of myoblasts post serum withdrawal to form myotubes. FIG. 3B, FIG. 3D, and FIG. 3F: Brightfield Imaging of myoblasts post serum withdrawal to form myotubes.

FIGS. 4A-4F show expression of GFP in C2C12 murine myoblasts transfected with NCT_GFP_020 plasmid. FIG. 4A. FIG. 4C, and FIG. 4E: GFP Signal of myoblasts post scrum withdrawal to form myotubes. FIGS. 4B, FIG. 4D, and FIG. 4F: Brightfield Imaging of myoblasts post serum withdrawal to form myotubes.

FIGS. 5A-SC show expression of enhanced green fluorescent protein (EGFP) in C2C12 murine myoblasts. Stable C2C12 cells were generated expressing EGFP and were mixed 50:50 with non-expressing C2C12 and fused on gelatin micro-molds to enhance fusion and alignment. Myotubes were imaged on day 8 post-serum withdrawal. FIG. 5A shows a construct in which the NLS is weaker as compared to the NES. FIG. 5B shows a construct in which the NLS is approximately the same strength as the NES. FIG. 5C shows a construct using NCT20, using a mutated SV40 NLS (SEQ ID NO: 3) and Alyref NES+Rev NuLS (SEQ ID NO: 1).

FIGS. 6A-6L show the results of an in vivo study using either construct 1: CBh promoter driving SV40 NLS-EGFP (PKKKRKV: SEQ ID NO: 5) or construct 2: CBh promoter driving NLS-EGFP-NES-NuLS (NCT20; SEQ ID NO: 11). Constructs were injected into tibialis anterior (TA) of C57BL6 mice at two different doses. TA fibers were harvested at either 3 or 8 weeks and imaged using fluorescence in situ hybridization against EGFP. Construct, dose, and time period are indicated on each figure. Imaging was performed using tile scans with 20× dry objective magnification.

FIGS. 7A-7H show the results of an in vivo study using either construct 1: CBh promoter driving SV40 NLS-EGFP (PKKKRKV; SEQ ID NO. 5) or construct 2: CBh promoter driving NLS-EGFP-NFS-NuLS (NCT20; SEQ ID NO: 11). Constructs were injected into tibialis anterior (TA) of C57BL6 mice at two different doses. TA fibers were harvested at either 3 or 8 weeks and imaged using fluorescence in sin hybridization against EGFP. Construct, dose, and time period are indicated on each figure. Imaging was performed using tile scans with 40× oil objective magnification.

FIGS. 8A-8J show DUX4 constructs and the results of in vitro experiments in C2C12 murine myoblasts cell lines. S+375-397 DUX4 construct contains HOX1 and HOX2 DNA binding domains but only part of the C-terminal domain (FIG. 8A). Identified by Mitsuhashi et al. as a potential inhibitory construct which can bind the DUX4 promoter without being toxic to cells (FIG. 8B). An S+375-397 dominant negative (SEQ ID NO: 12) and an S+375-397 dominant negative fused to ALYREF (S+375-397 dominant negative fused+ALYREF SEQ ID NO: 13) were used to inhibit a DUX4 promoter, greater inhibition was shown using the ALYREF sequence (FIGS. 8C-8J).

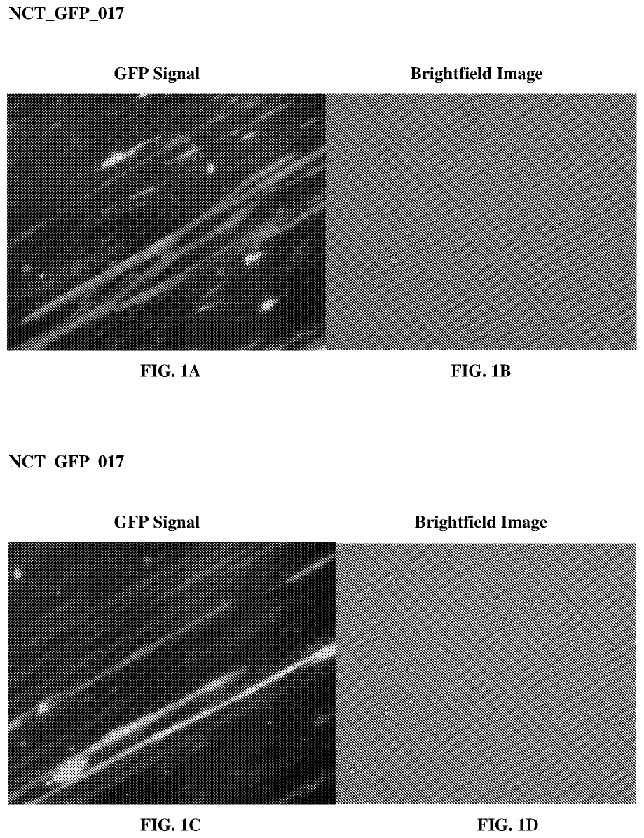

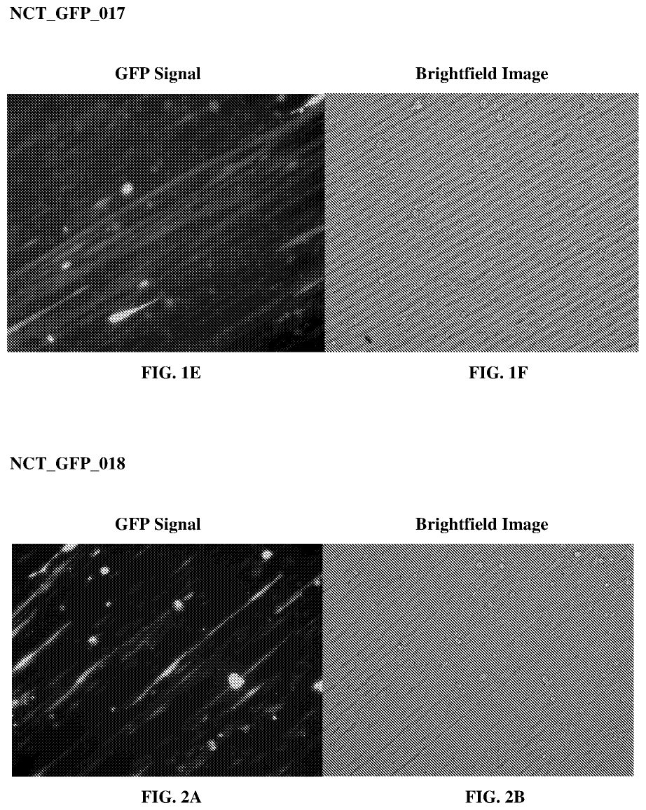

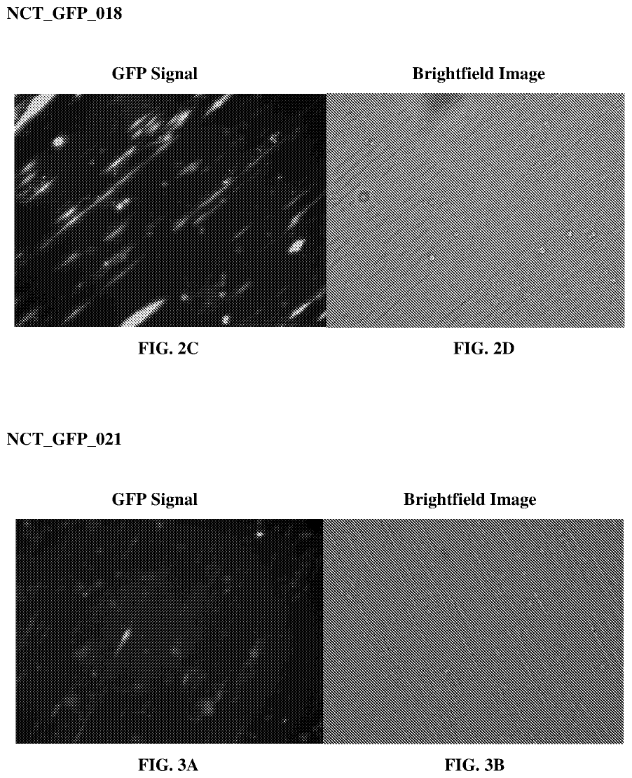

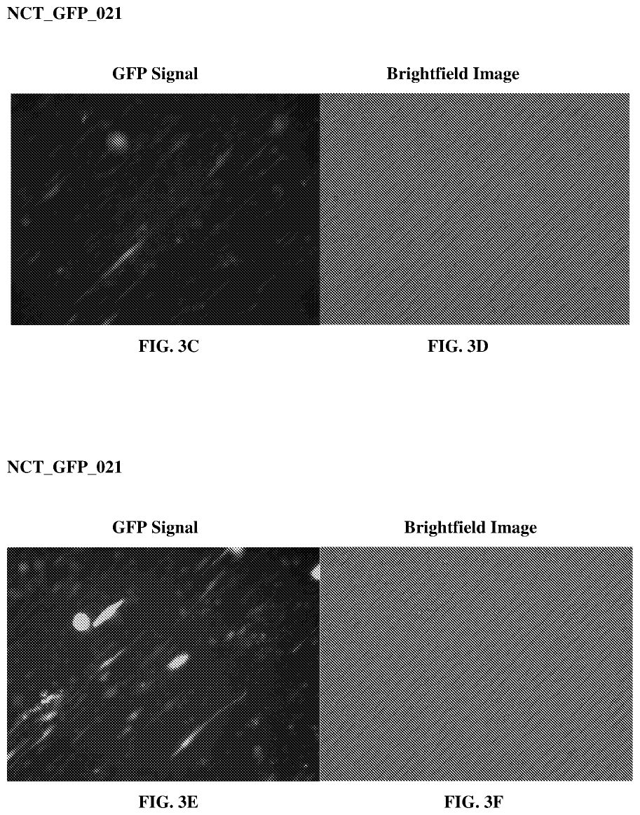

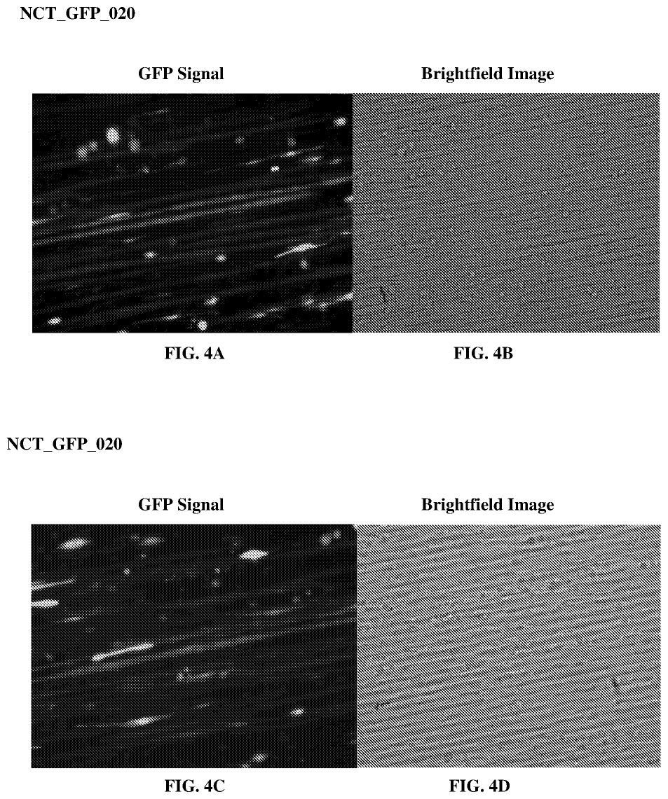

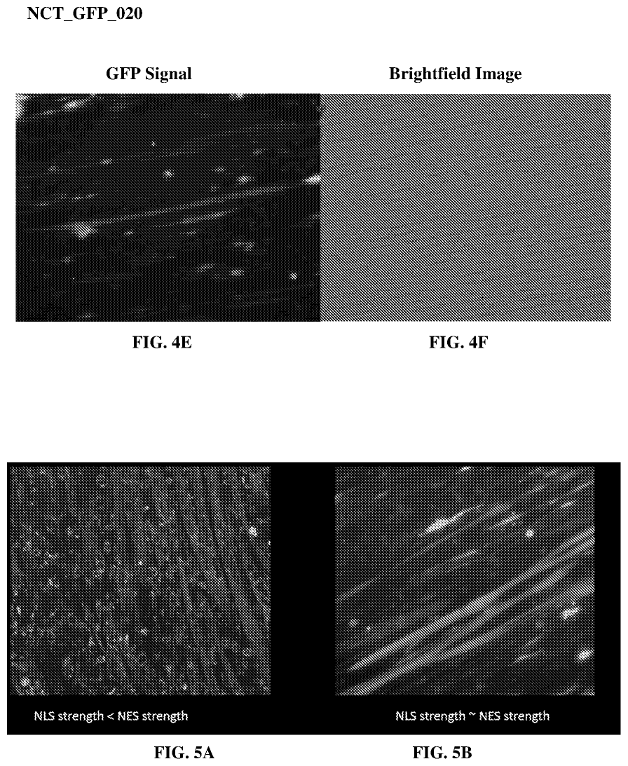

### DETAILED DESCRIPTION OF CERTAIN EMBODIMENTS

In some aspects, the disclosure relates to methods and compositions for delivering recombinant genes and/or fusion proteins to multinucleate cells. The terms “multinucleate,” “multinucleated,” and “polynuclear,” as may be used interchangeably herein, refer to a eukaryotic cell which has at least two (e.g., more than one) nuclei per cell (e.g., multiple nuclei (e.g., 2 or mare)) sharing one common cytoplasm. In some embodiments, the methods and compositions are useful for delivering a protein to a plurality of nuclei in a cell. In some embodiments, the disclosure relates to fusion proteins comprising one or more signals useful for promoting d-livery of the proteins to a plurality of nuclei in a multinucleate cell.

### Fusion Proteins

In some embodiments, a fusion protein comprises a protein of interest fused to one or more (e.g., 1, 2, 3, 4, 5, 6, 7, 8, 9, 10, or more) migration signals. The term “fusion protein,” as may be used herein, refers to a hybrid (e.g., chimeric, recombinant) polypeptide which comprises protein domains from at least two different proteins. A protein may comprise different domains, for example, a migration signal (e.g., NES, NLS, NoLS) and a protein of interest. Any of the fusion proteins provided herein may be produced by any method known in the art. For example, the proteins provided herein may be produced via recombinant protein expression and purification, which is especially suited for fusion proteins comprising a peptide linker. Methods for fusion protein expression and purification are well known, and include those described by Green and Sambrook. Molecular Cloning: A Laboratory Manual (4th ed., Cold Spring Harbor Laboratory Press. Cold Spring Harbor. N.Y. (2012)), the entire contents of which are incorporated herein by reference. A fusion protein can be encoded by a recombinant nucleic acid (e.g., DNA, RNA). In some embodiments, a fusion protein comprises one or more migration signals at its N-terminus (N-terminal migration signal(s). In some embodiments, a fusion protein comprises one or more migration signals at its C-terminus (C-terminal migration signal(s)). In some embodiments, a fusion protein comprises a protein of interest that is fused to only N-terminal migration signal(s). In some embodiments, a fusion protein contains N-terminal migration signals and does not contain C-terminal migrations signals. In some embodiments, a fusion protein contains C-terminal migration signals and does not contain N-terminal migration signals. In some embodiments, a fusion protein contains both N-terminal migration signals and C-terminal migration signals. In some embodiment, a fusion protein comprises a protein of interest that is fused to only C-terminal migration signal(s). In some embodiments, a fusion protein comprises a protein of interest that is fused to both N-terminal and C-terminal migration signal(s). In some embodiments, a fusion protein comprises one or more migration signals at the N-terminus. In some embodiments, a fusion protein comprises one or more migration signals at its C-terminus. In some embodiments, a fusion protein comprises a protein of interest fused to migration signal(s) only at the N-terminus. In some embodiments, a fusion protein comprises a protein of interest that is fused to only to migration signal(s) at the C-terminus. In some embodiments, a fusion protein comprises a protein of interest that is fused to migration signal(s) at both its N-terminus and C-terminus.

The term “protein of interest.” as may be used herein, refers to any protein (e.g., natural, synthetic, fragment, or variation thereof) which is the subject of (e.g., the target of) intervention. By this, the term is intended to represent the protein which is to be introduced into a system (e.g., organism, mammal, human, cell) to effect the environment into which it introduced (e.g., the system (e.g., organism, mammal, human, cell)). The protein of interest may be a naturally occurring protein. In such instances, the protein of interest may not be expressed by the subject, may be expressed in too little quantity, or a mutant may be expressed by the subject. Further, the protein may be a mutant or variant (e.g., non-naturally occurring, recombinant, engineered) of a protein. In such instances, the protein of interest may be not naturally found in the system (e.g., organism, mammal, human, cell) or may be introduced to alleviate a disease or disorder of the system (e.g., organism, mammal, human, cell). In some embodiments, the protein of interest is a naturally occurring protein. In some embodiments, the protein of interest is a mutant or variant (e.g., non-naturally occurring, recombinant, engineered) of a protein. In some embodiments, a protein of interest is a dominant negative variant (e.g., non-naturally occurring, recombinant, engineered) of a wild-type protein.

In some embodiments, the protein of interest may be a therapeutic protein. The term “therapeutic protein,” as may be used herein, refers to a protein which is intended to be used as, used as, and/or is a, treatment. The terms “treatment,” “treat,” and “treating,” as may be used interchangeably herein, refer to a clinical intervention aimed to reverse, alleviate, delay the onset of, or inhibit the progress of an, indication, disease, disorder, or one or more symptoms thereof. In some embodiments, treatment may be administered after one or more symptoms have developed and/or after a disease has been diagnosed. In other embodiments, treatment may be administered in the absence of symptoms (e.g., to prevent or delay onset of a symptom or inhibit onset or progression of a disease). For example, treatment may be administered to a susceptible individual (e.g., subject) prior to the onset of symptoms (e.g., in light of a history of symptoms and/or in light of genetic or other susceptibility factors). Treatment may also be continued after symptoms have resolved, for example, to prevent or delay their recurrence. In some embodiments, the therapeutic protein is intended to treat an indication, disease, or disorder of a multinucleate cell. In some embodiments, the therapeutic protein is intended to treat an indication, disease, or disorder of skeletal muscle. In some embodiments, the protein of interest is a nuclear protein. The term “nuclear protein.” as may be used herein, is intended to have the meaning as commonly attributed to it in the art. Generally, a nuclear protein refers to a protein found in the cell nucleus. In some embodiments, the protein of interest is a transcriptional factor, transcriptional repressor, RNA binding protein, DNA modifying protein (e.g., enzyme), DNA editing protein (e.g., enzyme), Cas protein (e.g., Cas9, Cas13, etc . . . ). In some embodiments, the protein of interest is DUX4. In some embodiments, a protein of interest is a variant of a wild-type protein. In some embodiments, a protein of interest is a DUX4 variant. In some embodiments, a DUX4 variant is a dominant negative variant. In some embodiment, a DUX 4 variant is a S+375-397 variant. In some embodiments the protein of interest may be usec to treat Facioscapulohumeral dystrophy. In son embodiments, the protein of interest may carry RNA (e.g. mRNA, miRNA (microRNA), shRNA (short hairpin RNA, small hairpin RNA), gRNA (guide RNA)). In some embodiments, the protein of interest may carry RNA to multiple nuclei. In some embodiments, a fusion protein comprises a protein of interest fused to a migration signal. The term “migration signal,” as may be used herein refers to peptides comprising amino acid sequences which facilitate migration of themselves (i.e., the peptide comprising the migration signal) and/or increase the residence time of a peptide or protein by means of affinity for an environment, non-affinity for another environment, or by the result of mechanical or chemical migration. Migration signals may also facilitate the migration of any peptide, protein, or other molecule (e.g., nucleic acid) they may be associated with (e.g., fused to: linked to; bound to by other mechanical, chemical, or biological interaction or bond). Generally, this is effectuated within a cell of an organism. The facilitation may occur as the result of recognition of the migration signal by import/export machinery within a cell or as the result of recognition and binding with other components which may be the target of migration (e.g., nucleic acids). In some embodiments, the migration signals facilitate nuclear export, nuclear localization, and/or nucleolar localization of a peptide. In some embodiments, the migration signals are nuclear export signals (NES), nuclear localization signals (NLS), and nucleolar localization signals (NoLS).

The terms “nuclear export signal, “nuclear export sequence,” and “NES.” as may be used interchangeably herein, refer to peptides (and their respective amino acid sequences) (generally 8-15 amino acid residues in length), usually attached to (e.g., part of) a protein, which promotes the protein for export from the cell nucleus to the cell cytoplasm through the nuclear pore complex using nuclear transport. In some embodiments, the NES peptide contains four hydrophobic residues. This is in contrast to the nuclear localization signal (NLS), which targets a protein located in the cytoplasm for import to the nucleus. The NES is recognized and bound by exportins. A common spacing of the hydrophobic residues found in an NES follows the following pattern LaaaLaaLaL, where “L” denotes a hydrophobic residue (often Leucine (“Leu” or “L”)) and “x” denotes any other amino acid. The spacing and configuration is believed to facilitate the protein's interaction with exportin. Various NES motifs are in the art and would be apparent to the skilled artisan. The terms also include, where context implies or requires, the nucleic acid sequences encoding the NES.

The terms “nuclear localization signal.” “nuclear localization sequence,” and “NLS” as may be used interchangeably herein, refer to peptides (and their respective amino acid sequences), usually attached to (e.g., part of) a protein, which promotes the protein import into the cell nucleus from the cell cytoplasm through the nuclear pore complex using nuclear transport. In some embodiments, an NLS often consists of one or more short peptides of positively charged amino acid residues of Lysine (“Lys” or “K”) or Arginine (“Arg” or “R”) exposed on the protein surface. This is in contrast to the nuclear export signal (NES), which targets a protein located in the nucleus for export to the cell cytoplasm. The NLS is recognized and bound by importins. Various NLS motifs are in the art and would be apparent to the skilled artisan. The spacing and configuration is believed to facilitate the protein's interaction with importin. The terms also include, where context implies or requires, the nucleic acid sequences encoding the NLS.

NLS can be further classified as either monopartite (i.e., of “one” part) or bipartite (i.e., of “two” parts). The classification as monopartite or bipartite results from the presence of a short spacer between the basis amino acid of a bipartite NLS, which is absent in a monopartite NLS.

The terms “nucleolar localization signal,” “NoLS,” and “Nucleolar Targeting Signal,” as may be used interchangeably herein, refer to peptides (and their respective amino acid sequences), usually attached to (e.g., part of) a protein, which promotes the protein localization to the cell nucleolus in the nucleus and promotes the protein's migration into the nucleolar compartment from the nucleoplasm. In some embodiments, an NoLS often comprises of primarily one or more short peptides of positively charged amino acid residues of Arginine (“Arg” or “R”) exposed on the protein surface. In some embodiments, an NoLS is a positively charged peptide. In some embodiments, an NoLS may have an isoelectric point above 12.6. In some embodiments, an NoLS is composed entirely of arginine residues. In some embodiments, an NoLS comprises at least 4 residues. In some embodiments, an NoLS comprises at least 5 residues. In some embodiments, an NoLS comprises at least 6 residues. In some embodiments, an NoLS comprises at least 4 (e.g., 4 or more) arginine residues. In some embodiments, an NoLS comprises at least 5 (e.g., 5 or more) arginine residues. In some embodiments, an NoLS comprises at least 6 (e.g., 6 or more) arginine residues. In some embodiments, an NoLS comprises at least 7 (e.g., 7 or more) arginine residues. In some embodiments, an NoLS comprises at least 8 (e.g., 8 or more) arginine residues. In some embodiments, an NoLS comprises at least 9 (e.g., 9 or more) arginine residues. In some embodiments, an NoLS consists of 9 arginine residues. In some embodiments, an NoLS comprises at least 10 (e.g., 10 or more) arginine residues. In some embodiments, an NoLS comprises at least 11 (e.g., 11 or more) arginine residues. In some embodiments, an NoLS comprises at least 12 (e.g., 12 or more) arginine residues. In some embodiments, an NoLS comprises at least 4 (e.g., 4 or more) arginine residues fused to an NLS. In some embodiments, an NoLS comprises a strong basic charge. In some embodiments, an NoLS comprises both arginine and lysine. In some embodiments, an NoLS comprises at least 4 (e.g., 4, 5, 6, 7, 8, 9, 10, 11, 12, or more) residues of arginine and/or lysine. In some embodiments, the NoLS is fused to an NLS. Motifs (e.g., peptides, sequences) involved in regulating nucleolar localization are not well defined and are believed to result from direct or indirect interaction with one of the nucleolar building blocks (e.g., rDNA, its transcripts, protein components, which facilitates localization. This is in contrast to NLS and/or NES, which serve as recognition motifs for binding with import/export machinery (respectively). In some embodiments, the NoLS includes an RNA binding motif. Various NLS motifs are in the art and would be apparent to the skilled artisan (for a more detailed background see for example, Martin et al., Principals of protein targeting to the nucleolus. Nucleus. 2015; 6(4): 314-325 (2015)), some exemplary NoLS can be found in Table 2. The terms also include, where context implies or requires, the nucleic acid sequences encoding the NoLS.

NES, NLS, and NoLS signals are known in the art and the skilled artisan will readily be able to ascertain and locate a vast number of such migration signals (see for example, Michael Bernhofer, Tatyana Goldberg, Silvana Wolf, Mohamed Ahmed, Julian Zaugg, Mikael Boden, Burkhard Rost, NLSdb—major update for database of nuclear localization signals and nuclear export signals, Nucleic Acids Research, Volume 46, Issue D1, 4 Jan. 2018, Pages D503-D508, //doi.org/10.1093/nar/gkx1021). For example, without limitation, various publications and resources exist which provide information on such signals (e.g., databases), such as NLSdb (rostlab.org/services/nlsdb1/browse.php) and NESdb (prodata.swmed.edu/LRNes/IndexFiles/namesGood.php), which (in addition to those listed in Berhofer hereinabove) without limitation, are contemplated as encompassed in this disclosure.

In sane embodiments, a fusion protein comprises a protein of interest and migration signals comprising, an NES and at least one migration signal selected from: (1) an NLS; and (2) an NoLS. In some embodiments, the fusion protein comprises migration signals comprising an NES and at least two migration signals selected from: (1) an NLS; and (2) an NoLS, wherein the at least two migration signals are distinct from one another. In some embodiments, the fusion protein comprises migration signals comprising an NES and at least two migration signals selected from: (1) an NLS; and (2) an NoLS, wherein the at least two migration signals are identical from one another. In some embodiments, the fusion protein may comprise multiple migration signals of the same type (e.g., more than one NES, more than one NLS, more than one NoLS). In some embodiments, the fusion protein may comprise multiple migration signals of the same type, but fewer than 20 migration signals (e.g., more than one, but less than 20, NES, more than one, but less than 20, NLS, more than one, but less than 20, NoLS). In some embodiments, the multiple migration signals of the same type may be identical (e.g., the same sequence). In some embodiments, the multiple migration signals of the same type may be non-identical (e.g., different sequences).

In some embodiments, a fusion protein comprises a protein of interest, wherein the protein of interest has properties of an NES, NLS, and/or NoLS. In some embodiments, a fusion protein comprises a protein of interest, wherein the protein of interest has properties of an NES, NLS, and/or NoLS, wherein the fusion protein further comprises at least one migration signal comprising an NES, an NLS, and/or an NoLS, wherein the at least one migration signal has properties distinct from those of the protein of interest. For clarity, without limitation, if a protein of interest has properties of an NES, an at least one migration signal may comprise an NLS and/or NoLS, but as can be envisioned by the skilled artisan, each combination and permutation of such a fusion protein are contemplated herein. In structuring a fusion protein in this way it is contemplated to exploit a fusion protein's inherent migration signal properties (e.g., as an NES, NILS, and/or NoLS) without the inclusion of an additional migration signal to incorporate the same property. It is well-known to the skilled artisan how to assess such properties of a protein of interest. For example, without limitation, such properties may be observed as a result of sequences in a protein known to be part of, or a whole, NES, NLS, or NoLS. Additionally, charges or other protein properties analogous or similar to those of an NES, NLS, and/or NoLS may be observed or measured. One of skill in the art will readily be able to ascertain proteins of this type without undue experimentation. For example, various assays exist to measure the nuclear localization, nucleolar localization, or cytoplasmic localization (e.g., as in the case of NES signals) activity of molecular components (e.g., sequences, nucleic acids, proteins). For example, and without limitation, heterokaryon assays for measurement and quantification of nucleucytoplasmic shuttling of proteins (see for example, McNicoll, F. and Müller-McNicoll. M. (2018). A Quantitative Heterokaryon Assay to Measure the Nucleocytoplasmic Shuttling of Proteins. Bio-protocol 8 (17): e2472. DOI: 10.21769/BioProtoc.2472; and Flach J. Bossie M. Vogel J, et al. A yeast RNA-binding protein shuttles between the nucleus and the cytoplasm. Mol Cell Biol. 1994; 14 (12): 8399-8407. doi: 10.1128/mcb.14.12.8399).

In some embodiments, the disclosure relates to a method of selecting proteins of interest for use in a fusion protein of the present disclosure, comprising, selecting a protein of interest based on inherent activity analogous to an NES, NLS, and/or NoLS. In some embodiments, a protein of interest is selected for having inherent properties of an NES, NLS, or NoLS, or a gene encoding a recombinant protein encoding the fusion is produced, and then attached to a migration signal. In some embodiments, a migration signal is distinct from the inherent NES, NLS, or NoLS property exhibited by, or quantified from, a protein of interest. In some embodiments, a fusion protein comprises a protein of interest selected for having inherent properties of an NES, NLS, or NoLS, and a migration signal. In some embodiments, a fusion protein comprises a protein of interest selected for having inherent properties of an NES, NLS, or NoLS, and a migration signal distinct from the inherent NES, NLS, or NoLS property exhibited by, or quantified from, the protein of interest. In some embodiments, an isolated nucleic acid encodes the fusion protein.

In some embodiments, the fusion protein comprises a protein of interest and migration signals comprising, an NES, an NLS, and an NoLS. In some embodiments, the fusion protein may comprise multiple migration signals of the same type (e.g., more than one NES, more than one NLS, more than one NoLS). In some embodiments, the multiple migration signals of the same type may be identical (e.g., the same sequence). In some embodiments, the multiple migration signals of the same type may be non-identical (e.g., different sequence).

In some embodiments, the fusion protein comprises a protein of interest, more than one NES (e.g., 2, 3, 4, 5, or more), and at least one migration signal selected from: (1) an NES; and (2) an NoLS. In some embodiments, the fusion protein comprises a protein of interest, at least one NES (e.g., 1, 2, 3, 4, 5, or more), and more than one NLS (e.g., 2, 3, 4, 5, or more). In some embodiments, the fusion protein comprises a protein of interest, at least one NES (e.g., 1, 2, 3, 4, 5, or more), and more than one NoLS (e.g., 2, 3, 4, 5, or more). In some embodiments, the fusion protein comprises a protein of interest, at least one NES (e.g., 1, 2, 3, 4, 5, or more), more than one NLS (e.g., 2, 3, 4, 5, or more), and an NoLS. In some embodiments, the fusion protein comprises a protein of interest, at least one NES (e.g., 10, 2, 3, 4, 5, or more), more than one NoLS (e.g., 2, 3, 4, 5, or morel, and an NLS. In some embodiments, the fusion protein comprises a protein of interest, at least one NES (e.g., 1, 2, 3, 4, 5, or more), more than one NLS (e.g., 2, 3, 4, 5, or more), and more than one NoLS (e.g., 2, 3, 4, 5, or mom). In some embodiments, the fusion protein comprises a protein of interest, more than one NES (e.g., 2, 3, 4, 5, or more), more than one NLS (e.g., 2, 3, 4, 5, or more), and more than one NoLS (e.g., 2, 3, 4, 5, or more).

In some embodiments, at least one NES of the fusion protein comprises a sequence with at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to a sequence selected from SEQ ID NO: 1-2 or 14-261. In some embodiments, at least one NES of the fusion protein comprises a sequence selected from SEQ ID NO: 1-2 or 14-261. In some embodiments, at least one NES of the fusion protein comprises a sequence of SEQ ID NO: 1, 2, or 14-261.

In some embodiments, at least one NLS of the fusion protein comprises a sequence with at least 80% identity (e.g., at least 80%, at least 81%, at least 82%, at least 83%, at least 4%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to a sequence selected from SEQ ID NO: 3-5 or 262-570. In some embodiments, at least one NLS of the fusion protein comprises a sequence selected from SEQ ID NO: 3-5 or 262-570. In some embodiments, at least one NLS of the fusion protein comprises a sequence of SEQ ID NO: 3-5 or 262-570.

In some embodiments, at least one NoLS of the fusion protein comprises a sequence with at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to a sequence selected from SEQ ID NO: 6. In some embodiments, at least one NoLS of the fusion protein comprises a sequence selected from SEQ ID NO: 6.

In some embodiments, a fusion protein comprises a protein of interest and one or more of an NES, NLS, and/or NoLS of Table 2, or a variant of any one thereof (e.g., having one or more, for example 1, 2, 3, 4, 5, 6, 7, 8, 9, or 10 amino acid insertions, deletions, and/or substitutions relative to the sequences listed in Table 2).

The terms “percent identity,” “sequence identity,” “% identity,” “% sequence identity,” and % identical,” as they may be interchangeably used herein, refer to a quantitative measurement of the similarity between two sequences (e.g., nucleic acid or amino acid). The percent identity of genomic DNA sequence, intron and exon sequence, and amino acid sequence between humans and other species varies by species type, with chimpanzee having the highest percent identity with humans of all species in each category. Percent identity can be determined using the algorithms of Karlin and Altschul. Proc. Natl. Acad. Sci. USA 87:2264-68, 1990, modified as in Karlin and Altschul, Proc. Natl. Acad. Sci. USA 90:5873-77, 1993. Such algorithms is incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences. Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at leas, 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (i.e., 0.1%), hundredths of a percent (i.e., 0.01%), etc.).

In some embodiments, at least one of the migration signals is positioned at the C-terminus of the protein of interest. In some embodiments, at least one of the migration signals is positioned at the N-terminus of the protein of interest. In some embodiments, at least one migration signal is positioned at each the N-terminus of the protein of interest and the C-terminus of the protein of interest. In some embodiments, all of the migration signals of the fusion protein are position at the N-terminus of the protein of interest. In some embodiments, all of the migration signals of the fusion protein are position at the C-terminus of the protein of interest.

In some embodiments, at least one of the migration signals is linked to the protein of interest via a linker. The term “linker.” as may be used herein, refers to a molecule linking two other molecules or moieties. Linkers are well known in the art and can comprise any suitable combination of nucleic acids or amino acids to facilitate the proper function of the structures they join. The linker can be a series of amino acids. The linker can be an amino acid sequence in the case of a linker joining two fusion proteins. For example, a protein (e.g., protein of interest) can be fused to a migration signal (e.g., NES, NLS, NoLS)(e.g., fusion protein as disclosed herein) by an amino acid linker sequence. The linker can also be a nucleotide sequence in the case of joining two nucleotide sequences together. In other embodiments, the linker is an organic molecule, group, polymer, or chemical moiety. In some embodiments, the linker is 1-100 amino acids in length, for example: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 30-35; 35-40; 40-45; 45-50; 50-60; 60-70; 70-80; 80-90; 90-100; 100-150; or 150-200 amino acids in length. In some embodiments, the inker is 5-1,000 nucleotides in length, for example: 5; 6; 7; 8; 9; 10; 11; 12; 13; 14; 15; 16; 17; 18; 19; 20; 21; 22; 23; 24; 25; 26; 27; 28; 29; 30; 30-35; 35-40; 40-45; 45-50; 50-60; 60-70; 70-80: 80-90; 90-100; 100-150; 150-200; 200-300; 300-500; 500-1,000; 1,000-2,000; or 2,000-5.000 nucleotides. Longer or shorter linkers are also contemplated. In some embodiments, at least one of the migration signal, is linked to at least one other migration signal via a linker. In some embodiments, the fusion protein comprises a linker between the protein of interest, at least one migration signal, wherein at least one migration signal is linked to at least one other migration signal via a linker. In some embodiments, the fusion protein comprises a linker between the protein of interest, at least one migration signal, wherein each migration signal positioned next to another migration signal is linked to the migration signal via a linker. In some embodiments, where all of the migration signals are positioned at one end of the protein of interest (e.g., at either the N-terminus of the protein of interest or the C-terminus of the protein of interest), they are linked to the protein of interest via a linker at such terminus (e.g., either the N-terminus of the protein of interest or the C-terminus of the protein of interest). In some embodiments, where migration signals are positioned at both ends of the protein of interest (e.g., at least one at the N-terminus of the protein of interest and at least one at the C-terminus of the protein of interest), at least one migration signal is linked to the protein of interest via a linker at such terminus (e.g., either the N-terminus or the C-terminus of the protein of interest). In some embodiments, where migration signals are positioned at both ends of the protein of interest (e.g., at least one at the N-terminus of the protein of interest and at least one at the C-terminus of the protein of interest), the migration signals at either terminus are linked to the protein of interest via a linker at such terminus (e.g., either the N-terminus or the C-terminus of the protein of interest).

In some embodiments, a fusion protein comprises a DUX4 protein sequence, or variant thereof. In some embodiments, a fusion protein comprises a DUX4 protein sequence of an S+375-397 dominant negative. In some embodiments, a fusion protein comprises a sequence with an S+375-397 dominant negative DUX4 protein sequence and an ALYREF sequence. In some embodiments, a fusion protein comprises a DUX4 protein sequence with at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9%) identity to SEQ ID NO: 13. In some embodiments, a fusion protein comprises a DUX4 protein sequence of SEQ ID NO: 13.

### Nucleic Acids

In some aspects, the disclosure relates to an isolated nucleic acid encoding at least one of the fusion proteins as disclosed herein. The term “isolated.” as may be used herein, refers to a characteristic of a material as provided herein (e.g., nucleic acid (e.g., RNA, DNA, polynucleotide), amino acid, peptide (e.g., polypeptide, protein), vector (e.g., viral vector (e.g., adeno-associated viral vector))), as being altered or removed from its natural state (e.g., native or original environment if it is naturally occurring) such material would otherwise be found. Therefore, a naturally-occurring nucleic acid or peptide present in a living animal is not isolated, but the same nucleic acid or peptide, separated by human intervention from some or all of the coexisting materials in the natural system, is “isolated.” For example, a nucleic acid or a peptide naturally present in a living animal is not “isolated,” but the same nucleic acid or peptide partially or completely separated from the coexisting materials of its natural state or host is “isolated.” An artificial, recombinant, or engineered material, for example, a non-naturally occurring nucleic acid construct or peptide construct, are, accordingly, also referred to as isolated. In some embodiments, an isolated nucleic acid is a recombinant nucleic acid in any environment. An isolated material can exist in substantially purified form, or can exist or; a non-native environment such as, for example, a vector or host cell, however, a material does not have to be purified in order to be isolated. Accordingly, a material may be part of a vector and/or part of a composition, and still be isolated in that such vector or composition is not part of the environment in which the material is found in its natural state.

In some embodiments, the isolated nucleic acid further comprises a promoter operably linked to the isolated nucleic acid encoding the fusion protein. The term “operably linked,” as may be used herein, refers to an arrangement of sequences or regions wherein the components are configured so as to perform their usual or intended function. Thus, a regulatory or control sequence operably linked to a coding sequence is capable of affecting the expression of the coding sequence. The regulatory or control sequences need not be contiguous with the coding sequence, so long as they function to direct the proper expression or polypeptide production.

Thus, as a non-limiting example, intervening untranslated but transcribed sequences can be present between a promoter sequence and the coding sequence and the promoter sequence can still be considered operably linked to the coding sequence. A promoter sequence, as described herein, is a DNA regulatory region a short distance from the 5′ end of a gene that acts as the binding site for RNA polymerase. The promoter sequence may bind RNA polymerase in a cell and/or initiate transcription of a downstream 3′ direction) coding sequence. The promoter sequence may be a promoter capable of initiating transcription in prokaryotes or eukaryotes. Some non-limiting examples of eukaryotic promoters include the cytomegalovirus (CMV) promoter, the chicken β-actin (CBA) promoter, and a hybrid form of the CBA promoter (CBh). In some embodiments, the promoter is a constitutive promoter, an inducible promoter, or a tissue specific promoter. In some embodiments, the promoter is a tissue specific promoter. In some embodiments, the tissue specific promoter is specific to skeletal muscle. In some embodiments, the tissue specific promoter is specific to liver tissue.

In some embodiments, the isolated nucleic acid further comprises at least one additional regulatory sequence. The terms “regulatory sequence,” “regulatory signal,” “control sequence,” and “control signal,” as may be used interchangeably herein, refer to sequences that are responsible for expressing a particular nucleic acid or may include other sequences, such as heterologous, synthetic, or partially synthetic sequences. The sequences can be of eukaryotic, prokaryotic, or viral origin that stimulate or repress transcription of a gene in a specific or non-specific manner and in an inducible or non-inducible manner. Regulatory or control regions may include origins of replication, RNA splice sites, introns, chimeric or hybrid introns, promoters, enhancers, transcriptional termination sequences, poly A sites, locus control regions, signal sequences that direct the polypeptide into the secretory pathways of the target cell, and introns. A heterologous regulatory region is not naturally associated with the expressed nucleic acid to which it is linked. Included among the heterologous regulatory regions are regulatory regions from a different species, regulatory regions from a different gene, hybrid regulatory sequences, and regulatory sequences that do not occur in nature, but which are designed by one of ordinary skill in the art.

In some embodiments, at least one NES of the fusion protein comprises a sequence with at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to a sequence selected from SEQ ID NO: 1-2 or 14-261. In some embodiments, at least one NES of the fusion protein comprises a sequence selected from SEQ ID NO: 1-2 or 14-261. In some embodiments, at least one NES of the fusion protein comprises a sequence of SEQ ID NO: 1-2 or 14-161.

In some embodiments, at least one NLS of the fusion protein comprises a sequence with at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to a sequence selected from SEQ ID NO: 3-5 or 262-570. In some embodiments, at least one NLS of the fusion protein comprises a sequence selected from SEQ ID NO: 3-5 or 262-570. In some embodiments, at least one NLS of the fusion protein comprises a sequence of SEQ ID NO: 3-5 or 262-570.

In some embodiments, at least one NoLS of the fusion protein comprises a sequence with at least 80% identity (e.g., at least 80%, at least 81%, at least 82%, at least 83%, at least F4%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, a least 98%, at least 98.5%, at least 99%, at least 99.5%, at least 99.6%, at least 99.7%, at least 99.8%, at least 99.9% identity) to a sequence selected from SEQ ID NO: 6. In some embodiments, at least one NoLS of the fusion protein comprises a sequence selected from SEQ ID NO: 6.

In some embodiments, an isolated nucleic acid encodes a fusion protein comprising a DUX4 protein sequence, or variant thereof. In some embodiments, an isolated nucleic acid encodes a fusion protein comprising a DUX4 protein sequence of an S+375-397 dominant negative. In some embodiments, an isolated nucleic acid encodes a fusion protein comprising a sequence with an S+375-397 dominant negative DUX4 protein sequence and an ALYREF sequence. In some embodiments, an isolated nucleic acid encodes a fusion protein comprising a DUX4 protein sequence with at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least, 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9%) identity to SEQ In NO: 13. In some embodiments, an isolated nucleic acid encodes a fusion protein comprising a DUX4 protein sequence of SEQ ID NO: 13.

### Viral Vectors

In some aspects, the disclosure relates to a viral vector comprising a recombinant virus and at least one of any of the isolated nucleic acids as described herein. In some embodiments, the recombinant virus is a recombinant adeno-associated virus (rAAV). In some embodiments, the recombinant virus is a recombinant lentivirus (rLV).

In same embodiments, the rAAV, comprises: (a) at least one of the isolated nucleic acids as described herein flanked by adeno-associated virus (AAV) inverted terminal repeats (ITRs); and (b) an AAV capsid protein.

Adeno-associated virus (AAV) is a small (20 nanometer (nm)) replication-defective, non-enveloped DNA virus, that depends on the presence of a second virus, for example, adenovirus or herpesvirus, for productive infection. AAV is not known to cause disease and induces a very mild immune response. AAV can infect both dividing and non-dividing cells and stably incorporates its genome into that of the host cell. AAV has a variety of different sub-types. e.g., scrotypes, and are known in the art. AAV vectors based on scrotype 2 (AAV2) provided a proof-of-concept for non-toxic and stable gene transfer in murine and large animal models. AAV vectors having distinct tissue targeting capabilities have been developed for gene therapy and research applications. AAV serotype affects tissue tropism of the respective viral particles and allows to target specific cell types or tissues, making AAV vectors attractive for in vivo gene delivery applications in which only a specific cell type or tissue is targeted and/or gene transfer into non-targeted cells or tissues is not desirable.

Wild-type (wt) AAV particles harbor a single-stranded DNA genome comprising two genes: the AAV rep gene and the AAV cap gene. The AAV rep gene encodes proteins controlling viral replication, structural gene expression, and integration into the host genome. The AAV cap gene encodes capsid structural proteins. The 5′ and 3′ termini of the AAV genome each comprise an inverted terminal repeat (ITR) region, which is involved in multiplication of the AAV genome. In some embodiments, an AAV ITR sequence comprises 145 nucleotides. In general, an AAV ITR sequence is a self-complementary nucleic acid structure that is able to form a hairpin, which plays a role in AAV self-priming for synthesis of the second DNA AAV strand during the viral life cycle. Recombinant AAV (rAAV) vectors am generally produced by replacing the viral genes, or parts thereof, with a heterologous expression cassettes. The term “expression cassette,” as may be used herein, refers to a nucleic acid construct comprising nucleic acid elements sufficient for the expression of a gene product. Typically, an expression cassette comprises a nucleic acid encoding a gene product operably linked to a promoter sequence. In some embodiments, the expression cassette of the rAAVs described herein is any of the isolated nucleic acids of the present disclosure.

Typically, rAAV genomes up to about 5 kb in length can efficiently be packaged into infectious viral particles useful for gene transfer. In some embodiments, the rAAV construct is a single-stranded rAAV construct (ssAAV). That is, the rAAV construct contains two ITRs, a 5′ ITR and a 3′ ITR that comprise a functional terminal resolution sites (TRS) each. In some such embodiments, the AAV construct is a double-stranded, self-complementary AAV (scAAV) construct. For an overview of AAV biology. ITR function, and scAAV constructs, see McCarty D M. Self-complementary AAV vectors; advances and applications. Mol Thor. 2008 October; 16(10): at pages 1648-51, first full paragraph, incorporated herein by reference for disclosure of AAV and scAAV constructs. ITR function, and role of TRS ITR in scAAV constructs. An rAAV vector comprising a TRS ITR cannot correctly be nicked during the replication cycle and, accordingly, produces a self-complementary, double-stranded AAV (scAAV) genome, which can efficiently be packaged into infectious AAV particles. Various rAAV, ssAAV, and scAAV vectors, as well as the advantages and drawbacks of each class of vector for specific applications and methods of using such vectors in gene transfer applications are well known to those of skill in the art (see, for example. Choi V W, Samulski R J. McCarty D M. Effects of adeno-associated virus DNA hairpin structure on recombination. J Virol. 2005 June; 79(11):6801-7; McCarty D M. Young S M Jr. Samulski R J. Integration of adeno-associated 20 virus (AAV) and recombinant AAV vectors. Annu Rev Genet. 2004; 38:819-45: McCarty D M. Monahan P E. Samulski R J. Self-complementary recombinant adeno-associated virus (scAAV) vectors promote efficient transduction independently of DNA synthesis. Gene Ther. 2001 August; 8(16):1248-54; and McCarty D M. Self-complementary AAV vectors: advances and applications. Mot Ther. 2008 October; 16(10):1648-56; all references cited in this application are incorporated herein by reference for disclosure of AAV, rAAV, and scAAV vectors).

In some embodiments, rAAV vectors are engineered to target specific cells, cell types, or tissues, for example, skeletal muscle or liver tissue. The AAV sequences of a rAAV construct provided herein typically comprise the cisacting 5′ and 5′ inverted terminal repeat sequences (See. e.g., B. J. Carter, in “Handbook of Parvoviruses”, ed., P. Tijsser, CRC Press. pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). AAV ITR sequences may be obtained from any known AAV, including presently identified mammalian AAV types. In addition to the major elements identified above for the rAAV vectors and constructs, a rAAV vector may also include additional transcriptional control elements. Transcriptional control elements are known to those of skill in the art and exemplary elements include transcription initiation, termination, promoter and enhancer sequences. RNA processing signals such as splicing and polyadenylation (polyA) signals, sequences that stabilize cytoplasmic mRNA, sequences that enhance translation efficiency (e.g., Kozak consensus sequences), sequences that enhance protein stability, and, if appropriate, sequences that enhance secretion of the encoded product. A great number of expression control sequences, including promoters which are native, constitutive, inducible and/or tissue-specific, are known in die art and may be utilized. Adeno-associated viral ITR sequences may be obtained from any known AAV, including presently identified mammalian AAV types.

In some embodiments, the AAV capsid protein is selected from an AAV having a serotype of: AAV1, AAV2, AAV3, AAV3b, AAV4, AAV5, AAV6, AAV7, AAV8, AAVrh.8, AAV9, AAV10, AAVrh.10, AAVrh.39, AAVrh.43, AAV.PHPB, AAV.PHPB.e. AAVrh32.33, or a variant thereof. In some embodiments, the AAV capsid protein is of serotype AAV1. In some embodiments, the AAV capsid protein is of serotype AAV2. In some embodiments, the AAV capsid protein is of scrotype AAV3. In some embodiments, the AAV capsid protein is of serotype AAV3b. In some embodiments, the AAV capsid protein is of serotype AAV4. In some embodiments, the AAV capsid protein is of scrotype AAV5. In some embodiments, the AAV capsid protein is of serotype AAV6. In some embodiments, the AAV capsid protein is of serotype AAV7. In some embodiments, the AAV capsid protein is of serotype AAV8. In some embodiments, the AAV capsid protein is of serotype AAVrh.8. In some embodiments, the AAV capsid protein is of serotype AAV9. In some embodiments, the AAV capsid protein is of serotype AAV10. In some embodiments, the AAV capsid protein is of serotype AAVrh.10. In some embodiments, the AAV capsid protein is of serotype AAVrh.39. In some embodiments, the AAV capsid protein is of serotype AAVrh.43. In some embodiments, the AAV capsid protein is of serotype AAV.PHPB. In some embodiments, the AAV capsid protein is of serotype AAV.PHPB.e. In some embodiments, the AAV capsid protein is of serotype AAVrh32.33.

In some embodiments, the AAV capsid protein exhibits a tropism for tissue having multinucleate cells. In some embodiments, the AAV capsid protein exhibits a tropism for multinucleate cells. In some embodiments, the AAV capsid protein exhibits a tropism for skeletal muscle. In some embodiments, the AAV capsid protein serotype is selected from: AAV1; AAV6; AAV7; AAV8; and AAV9.

### Compositions

In some aspects, the disclosure relates to a composition comprising at least one of the fusion proteins as described herein, at least one of the isolated nucleic acids as described herein, or at least one of the rAAVs as disclosed herein, and a pharmaceutically acceptable excipient.

In some embodiments, at least one of the fusion proteins as described herein, at least one of the isolated nucleic acids as described herein, or at least one of the rAAVs as disclosed herein can be formulated for administration to a subject as a composition, which, as used herein comprises at least one of the fusion proteins as described herein, at least one of the isolated nucleic acids as described herein, or at least one of the rAAVs as disclosed herein and a pharmaceutically acceptable carrier, diluent, or excipient. A carrier, diluent, or excipient that is “pharmaceutically acceptable” includes one that is sterile and pyrogen free. Suitable pharmaceutical carriers, diluents, and excipients are well known in the art. The carrier(s) should be “acceptable” in the sense of being compatible with the at least one of the fusion protein, at least one isolated nucleic acid, or at least one rAAV and not deleterious to the recipients thereof.

Formulations suitable for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. Aqueous solutions may be suitably buffered (preferably to a pH of from about 3 to about 9). The preparation of suitable parenteral formulations under sterile conditions is readily accomplished by standard pharmaceutical techniques well-known to those skilled in the art.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the fusion protein, nucleic acid, rAAV and/or rLV and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the agents, can be dissolved and administered in a pharmaceutical excipient such as Water-for-Injection, 0.9% saline, or 5% glucose solution.

Any of the fusion proteins, nucleic acids, rAAVs, and/or rLVs disclosed herein may be administered by any administration route known in the art, such as parenteral administration, oral administration, buccal administration, sublingual administration (e.g., tablets, capsules, ovules, elixirs, solutions or suspensions, which may contain flavoring or coloring agents, for immediate-, delayed- or controlled-release applications) topical administration, or inhalation, in the form of a pharmaceutical formulation (e.g., comprising a composition) comprising the active ingredient, optionally in the form of a non-toxic organic, or inorganic, acid, or base, addition salt, in a pharmaceutically acceptable dosage form. Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the fusion proteins, nucleic acids, rAAVs, and/or rLVs of the disclosure may be combined with various sweetening or flavoring agents, coloring matter or dyes, with emulsifying and/or suspending agents and with diluents such as water, ethanol, propylene glycol and glycerin, and combinations thereof. Additionally, lubricating agents such as magnesium stearate, stearic acid, glyceryl behenate and tale may be included.

The formulations may be presented in unit-dose or multi-dose containers, for example scaled ampoules or vials, and may be stored in a freeze-dried (lyophilized) condition requiring only the addition of the sterile liquid carrier immediately prior to use.

In some embodiments, any of the fusion proteins, nucleic acids, rAAVs, and/or rLVs described herein can be administered to a subject in single or divided doses. In some embodiments, any of the fusion proteins, nucleic acids, rAAVs, and/or rLVs described herein is administered to a subject in a single dose. In some embodiments, any of the fusion proteins, nucleic acids, rAAVs, and/or rLVs described is administered to a subject in divided doses (e.g., multiple or sequential doses). In some embodiments, any of the fusion proteins, nucleic acids, rAAVs, and/or rLVs described herein can be administered to a subject at a dose of between 25 and 100 μg per subject or between 0.4 to 1.7 μg/kg per subject, administered in single or divided doses (e.g., multiple or sequential doses). A physician in any event may determine the actual dosage which will be most suitable for any subject, which will vary with the age, weight, and the particular indications (e.g., disease or disorder) to be treated or prevented.

### Methods

In some aspects, the disclosure relates to a method of delivering at least one of the fusion proteins as disclosed herein, to multinucleate cells. Various examples of multinucleate cells exist throughout nature and are well known to the skilled artisan. Multinucleate cells can occur as part of a natural development, for example cells of the mammalian placenta, skeletal muscle, liver tissue, and osteoclasts of bone tissue.

In some embodiments, the multinucleate cells are mammalian. In some embodiments, the multinucleate cells are cells of the placenta. In some embodiments, the multinucleate cells are skeletal muscle cells. In some embodiments, the multinucleate cells are cells of the liver (e.g., hepatocytes). In some embodiments, the multinucleate cells are osteoclasts. In some embodiments, the multinucleate cells are human cells.

Multinucleate cells can also occur as the result of fusion of cells or as the result of exposure to a pathogen, for example Human Immuno-deficiency Virus (HIV). In some embodiments, the multinucleate cells are cells resulting from fusion of cells. In some embodiments, the multinucleate cells am cells resulting from exposure to a pathogen. In some embodiments, the multinucleate cells are cells resulting from exposure to IV.

In some aspects, the method comprises, administering at least one of the fusion protein as disclosed herein, at least one of the isolated nucleic acids as disclosed herein, at least one of the rAAVs as disclosed herein, at least one of the rLVs as disclosed herein, or at least one of the compositions as disclosed herein, to a subject.

In some aspects, the disclosure relates to a method of administering the fusion proteins, compositions, and/or isolated nucleic acids of the disclosure to a multinucleate tissue in a subject. In some embodiments, administration is directly to the tissue. In some embodiments, administration is by intravenous administration.

The term “subject,” as used herein, refer to any organism in need of treatment or diagnosis using the subject matter herein. For example without limitation, subjects may include mammals and non-mammals. As used herein, a “mammal,” refers to any animal constituting the class Mammalia (e.g., a human, mouse, rat, cat, dog, sheep, rabbit, horse, cow, goat, pig, guinea pig, hamster, chicken, turkey, or a non-human primate (e.g., Marmoset, Macaque)). In some embodiments, the subject in any of the methods as described herein is a mammal. In some embodiments, the subject is human.

In some embodiments, the subject has a disorder. In some embodiments, the disorder is a genetic disorder. In some embodiments, the disorder is muscular dystrophy. In some embodiments, the disorder is a genetically dominant muscle disorder. In some embodiments, the disorder is oculopharyngeal muscular dystrophy. In some embodiments, the disorder is Emery-Dreifuss muscular dystrophy. In some embodiments, the disorder is myotonic dystrophy. In some embodiments, the disorder is Facioscapulohumeral dystrophy. In some embodiments, the disorder is Charcot-Marie Tooth disease. In some embodiments, the disorder is Limb Girdle muscular dystrophy. In some embodiments, the disorder is Duchenne muscular dystrophy. In some embodiments, the disorder is a muscular dystrophy. To perform such a method, an effective amount of the fusion proteins, nucleic acids, rAAVs, and/or rLVs as described herein can be administered as described herein. The terms “effective amount,” “therapeutically effective amount,” and “pharmaceutically effective amount,” as may be used interchangeably herein, refer to an amount of the fusion proteins, nucleic acids, rAAVs, and/or rLVs sufficient to elicit a desired biological response. For example, in some embodiments, an effective amount of a fusion protein, nucleic acid, rAAV, and/or rLV may refer to the amount of fusion proteins, nucleic acids, rAAVs, and/or rLVs sufficient to treat the disorder. The terms “treatment,” “treat,” and “treating.” as may be used interchangeably herein, refer to a clinical intervention aimed to reverse, alleviate, delay the onset of, or inhibit the progress of a, indication, disease, disorder, or one or more symptoms thereof, as described herein. In some embodiments, treatment may be administered after one or more symptoms have developed and/or after a disease has been diagnosed. In other embodiments, treatment may be administered in the absence of symptoms (e.g., to prevent or delay onset of a symptom or inhibit onset or progression of a disease). For example, treatment may be administered to a susceptible individual (e.g., subject) prior to the onset of symptoms (e.g., in light of a history of symptoms and/or in light of genetic or other susceptibility factors). Treatment may also be continued after symptoms have resolved, for example, to prevent or delay their recurrence. As will be appreciated by the skilled artisan, the effective amount may vary depending on various factors as, for example, on the desired biological response (e.g., on the miRNA to be inhibited, the mRNA transcript to be promoted), on the cell or tissue being targeted, and on the agent, being used.

In some embodiments, any of the isolated nucleic acids of the disclosure, any of the fusion proteins of the disclosure, any of the vectors of the disclosure, and/or any of the compositions of the disclosure are administered intramuscularly. In some embodiments, any of the isolated nucleic acids of the disclosure, any of the fusion proteins of the disclosure, any of the vectors of the disclosure, and/or any of the compositions of the disclosure are administered intrathecally. In some embodiments, any of the isolated nucleic acids of the disclosure, any of the fusion proteins of the disclosure, any of the vectors of the disclosure, and/or any of the compositions of the disclosure are administered intravenously. In some embodiments, any of the isolated nucleic acids of the disclosure, any of the fusion proteins of the disclosure, any of the vectors of the disclosure, and/or any of the compositions of the disclosure are administered by retrograde intravenous administration. In some embodiments, the composition administered intravascularly. In some embodiments, any of the isolated nucleic acids of the disclosure, any of the fusion proteins of the disclosure, any of the vectors of the disclosure, and/or any of the compositions of the disclosure are administered systemically. In some embodiments, any of the isolated nucleic acids of the disclosure, any of the fusion proteins of the disclosure, any of the vectors of the disclosure, and/or any of the compositions of the disclosure are administered intra-arterially. In some embodiments, any of the isolated nucleic acids of the disclosure, any of the fusion proteins of the disclosure, any of the vectors of the disclosure, and/or any of the compositions of the disclosure are administered by intraportal administration. In some embodiments, any of the isolated nucleic acids of the disclosure, any of the fusion proteins of the disclosure, any of the vectors of the disclosure, and/or any of the compositions of the disclosure are administered by intratracheal administration. In some embodiments, any of the isolated nucleic acids of the disclosure, any of the fusion proteins of the disclosure, any of the vectors of the disclosure, and/or any of the compositions of the disclosure are administered by direct injection.

In some embodiments, the composition is formulated for intramuscular administration. In some embodiments, the composition is formulated for intrathecal administration. In some embodiments, the composition is formulated for intravenous administration. In some embodiments, the composition is formulated for retrograde intravenous administration. In some embodiments, the composition is formulated for intravascular administration. In some embodiments, the composition is formulated for systemic administration. In some embodiments, the composition is formulated for intra-arterial administration. In some embodiments, the composition is formulated for intraportal administration. In some embodiments, the composition is formulated for intratracheal administration. In some embodiments, the composition is formulated for direct injection.

### EXAMPLES

### Example 1: Inclusion of a Nucleolar Localization Sequence (NoLS) Increases Transduction of Multiple Nuclei in Multi-Nucleated Cells

C2C12 mouse myoblasts were transfected with the experimental GFP plasmid constructs and allowed to fuse. Due to the inefficient nature of transient transfection and the fact that many nuclei never receive the plasmid, imaging of GFP distribution within the fused tubes reveals the ability of each construct to spread throughout the myotubes. Myotubes were imaged after several days of fusion in culture.

TABLE 1
Experimental Plasmid Constructs

| | | NLS | | NES | NoLS |
|---|---|---|---|---|---|
| | NLS | SEQ ID | NES | SEQ ID | SEQ ID |
| Plasmid ID | Strength | NO: | Strength | NO: | NO: |
| NCT_GFP_017 | Weak | 1 | ALYREF | 3 | N/A |
| NCT_GFP_018 | Strong | 2 | ALYREF | 3 | N/A |
| NCT_GFP_020 | Weak | 1 | ALYREF | 3 | 5 |
| NCT_GFP_021 | Strong | 2 | Weak P53 | 4 | N/A |

### Example 2: Relative Strength of Signal Tags Affects Expression In Vitro

Stable C2C12 were generated in which EGFP is stably expressed. Various Nuclear Localization Sequences (NLS) or Nuclear Export Sequences (NES) were attached to EGFP (an EF1alpha promoter was used to drive EGFP expression). Cells were mixed 50:50 with non-expressing C2C12 cells and fused on gelatin micro-molds to enhance fusion and alignment

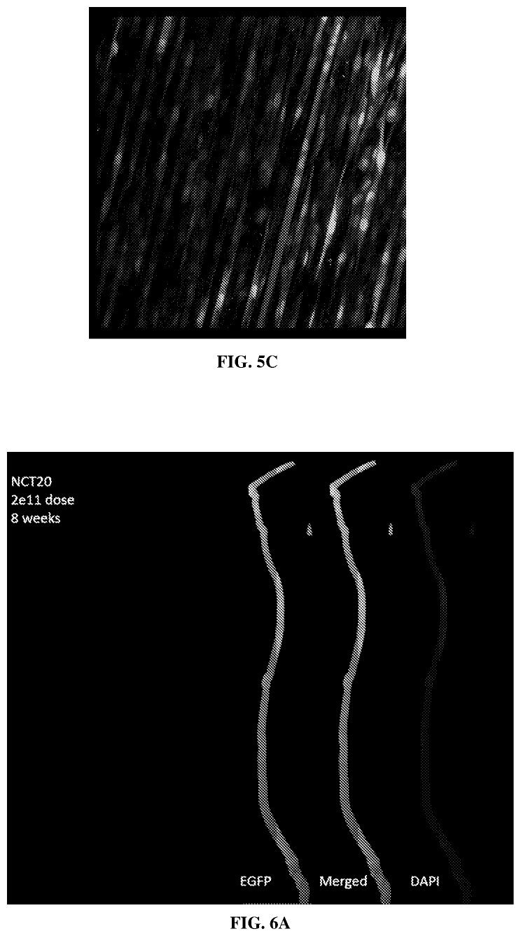

Myotubes were imaged on day 8 post-serum withdrawal (FIGS. 5A-5C). Various constructs were imaged, constructs where the NLS was weaker than the NES (FIGS. 5A and 5C) and where the NLS was approximately equal strength to the NES (FIG. 5B). As is shown, where the NLS was weaker than the NES, increased proliferation of EGFP among the nuclei was observed (FIGS. 5A and 5C) relative to equal strength tags (FIG. 5B).

### Example 3: Adeno-Associated Viral Constructs Transduce Multinucleated Cells In Vivo

Two constructs were made: construct 1: CBh promoter driving SV40 NLS-EGFP (PKKKRKV (SEQ ID NO: 5)); and construct 2: CBh promoter driving NLS-EGFP-NES-NuLS (NCT20 (SEQ ID NO: 11)). Each used an AAV6 capsid protein.

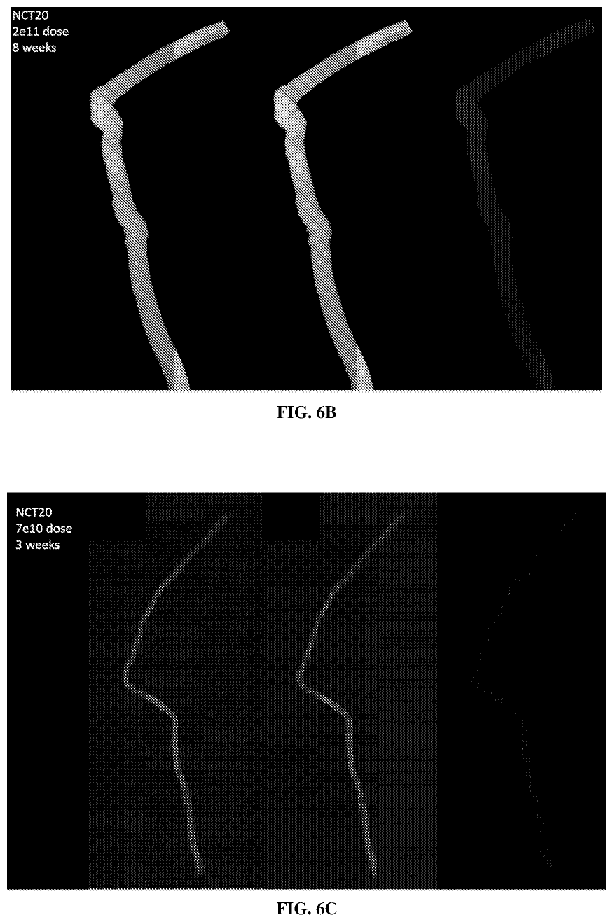

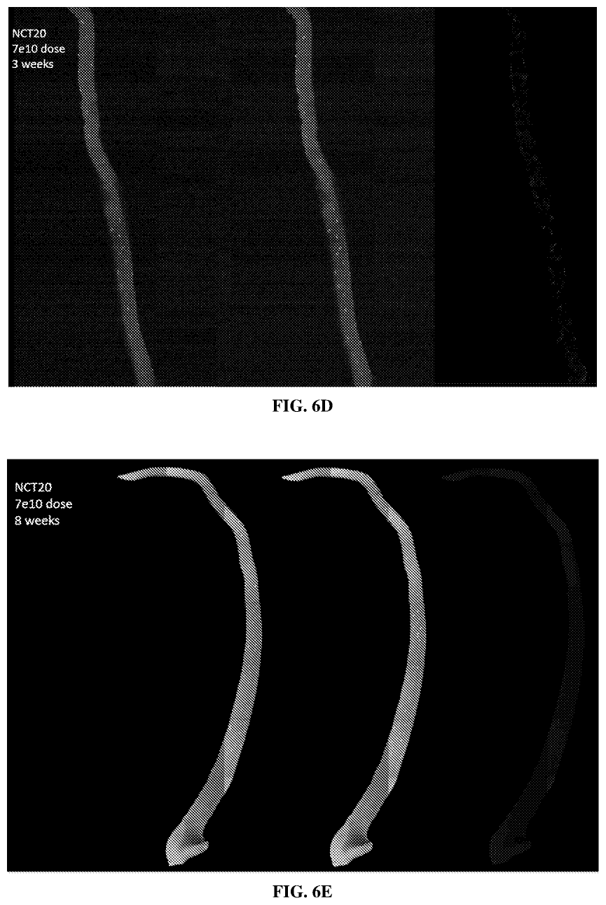

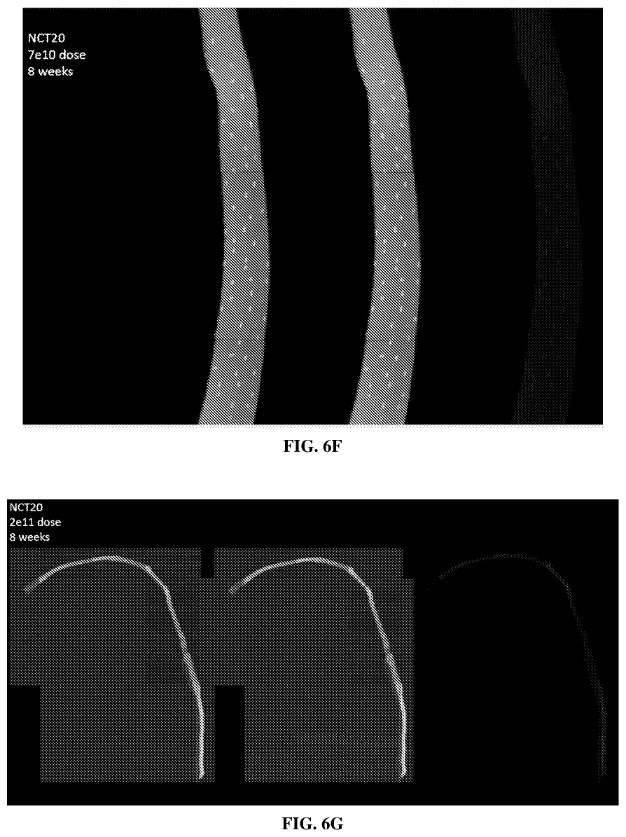

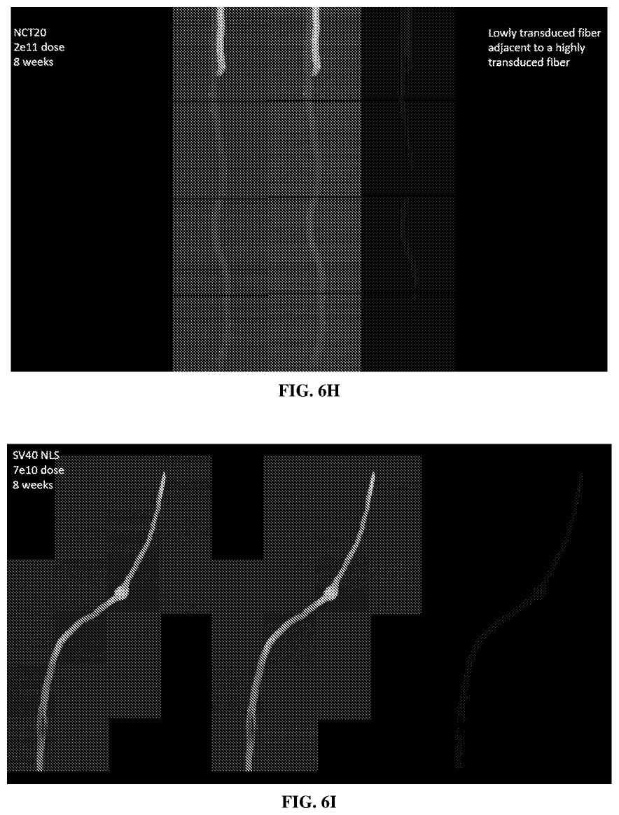

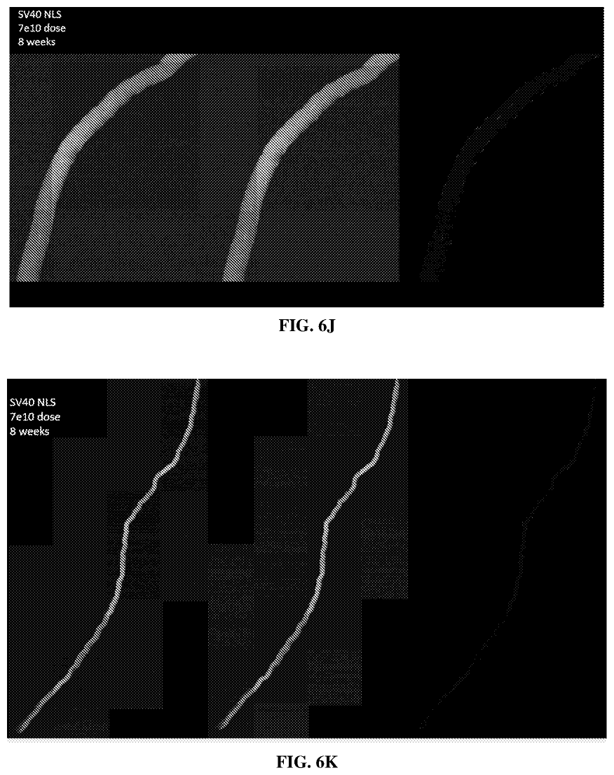

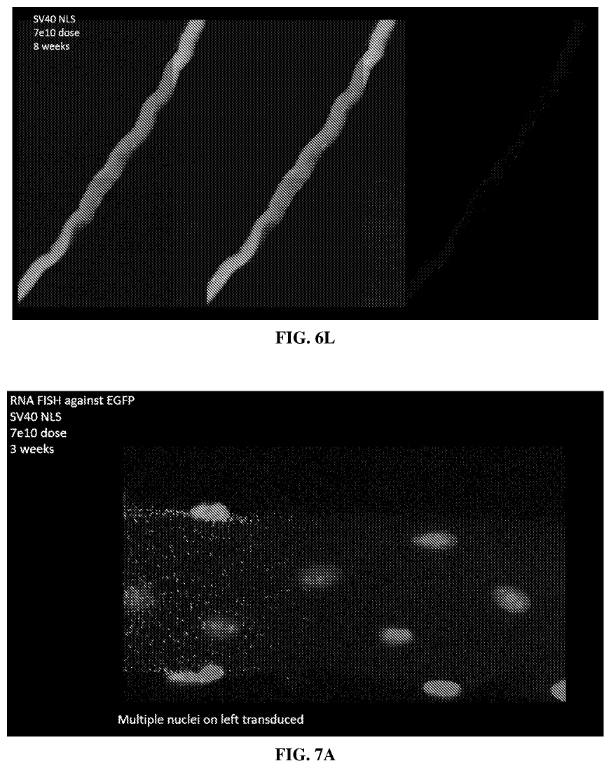

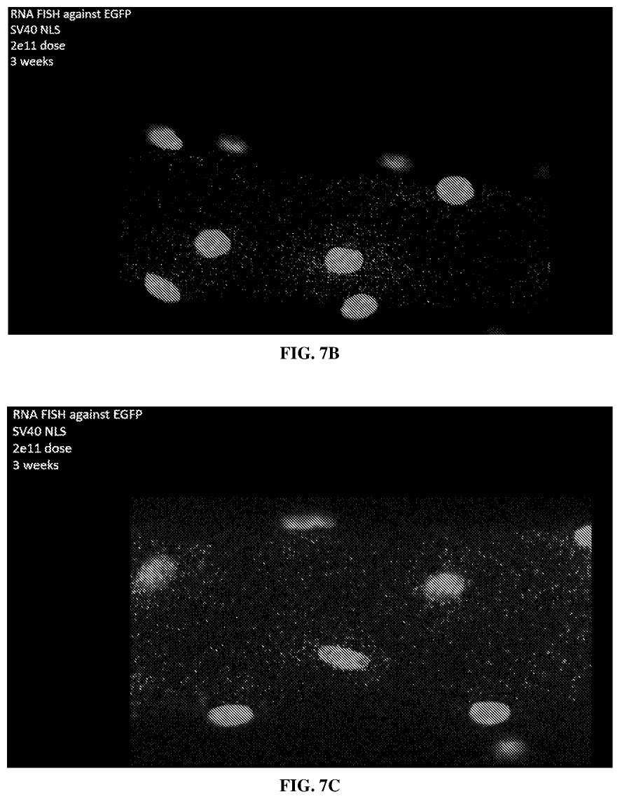

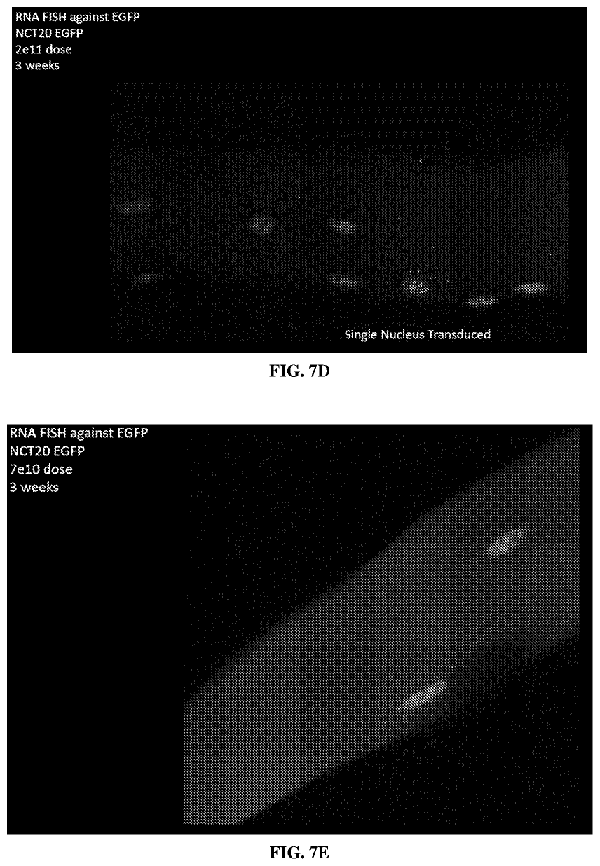

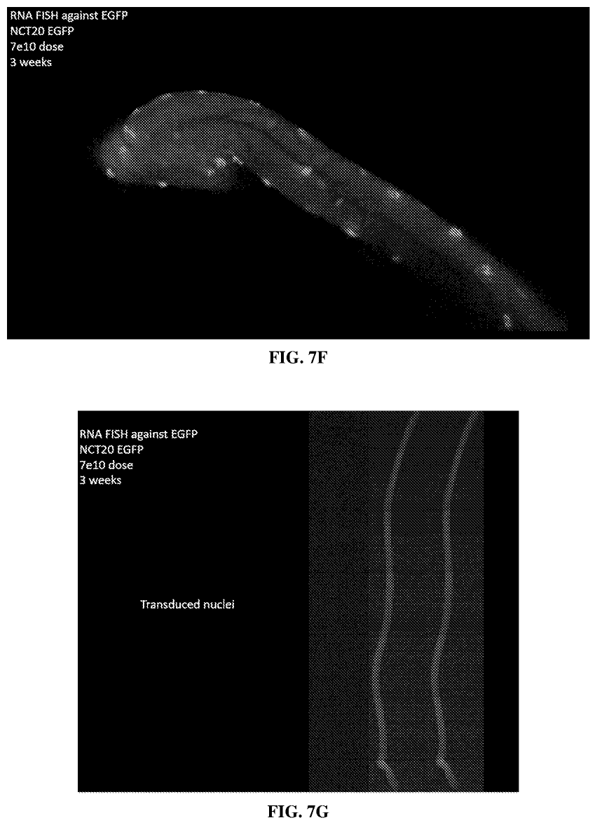

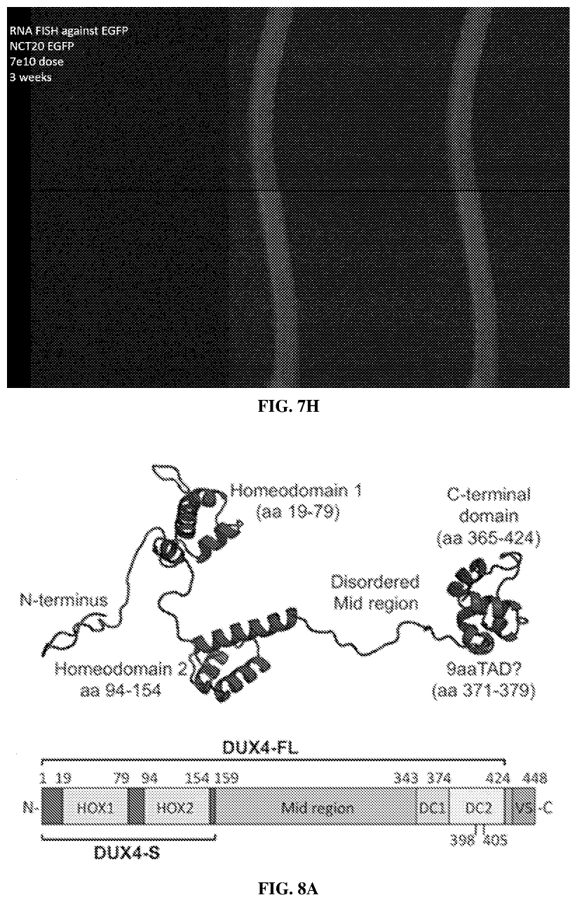

The constructs were administered in two doses, either 7×10\hat{ }10 viral genome (vg) total or 2×10\hat{ }11 vg total. Doses were delivered via intramuscular injection into the tibialis anterior (TA) of C57BL6 mice. Tibialis Anterior fibers were harvested at two time periods, either 3 weeks or 8 weeks post injection. Fibers were analyzed using immunofluorescence and fluorescent in situ hybridization (FISH) using RNA ainst EGFP (FIGS. 6A-6L and FIGS. 7A-7H). Native EGFP was also imaged for some fibers. Fibers were assessed for EGFP expression and stained using DAPI (FIGS. 6A-6L, EGFP shown in leftmost panels/fibers; DAPI shown in rightmost panels/fibers: merged in center panels/fibers), as well as using FISH (FIGS. 7A-7H). Highly and lowly transduced fibers observed in both dosage groups.

### Example 4. Application of Nuclear Spreading to Facioscapulohumeral Muscular Dystrophy (FSHD)

Facioscapulohumeral muscular dystrophy (FSHD) is a muscular dystrophy characterized by weakness of facial muscles, scapula, shoulder blades, upper arms, other muscles. Broad range of disease severity, with muscle weakness often presenting asymmetrically, which is caused by aberrant expression of DUX4 in muscle.

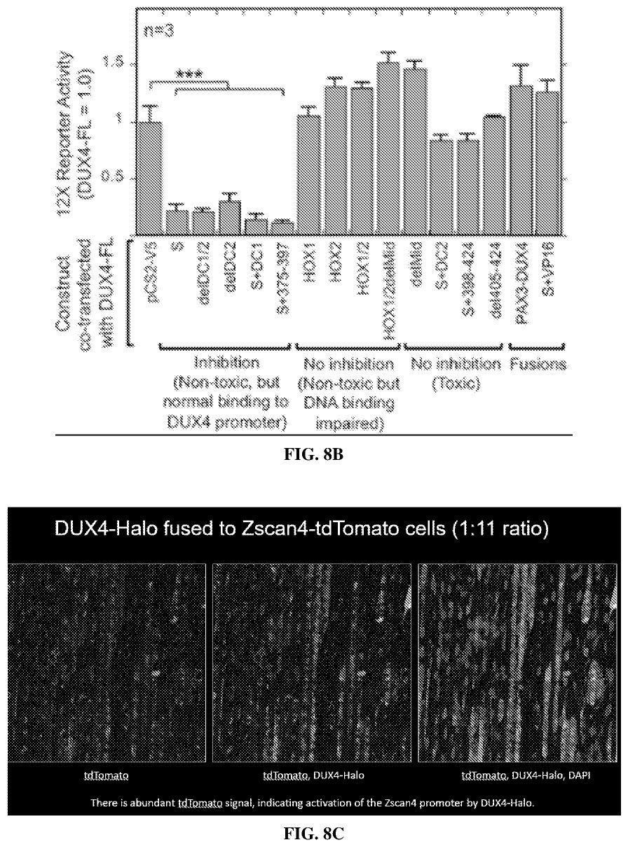

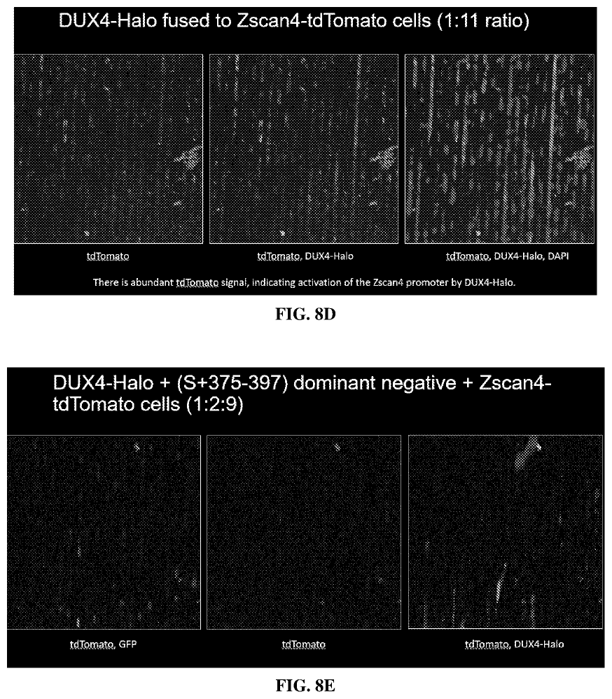

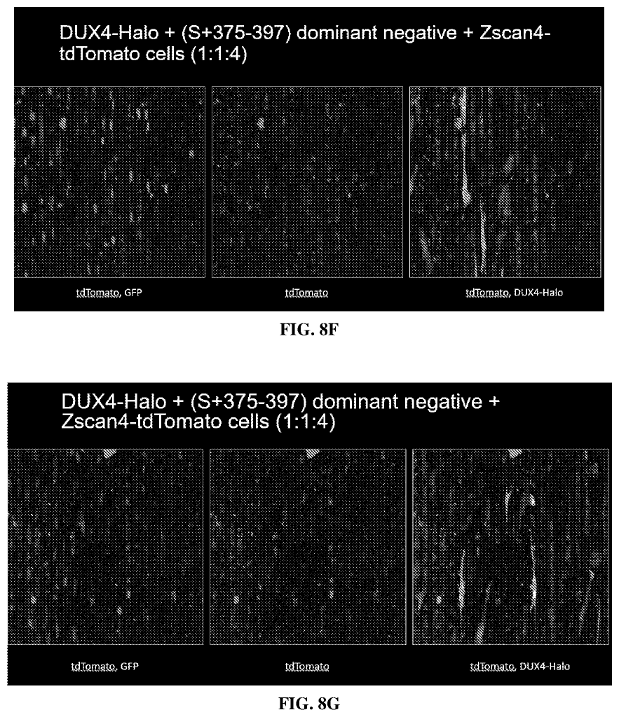

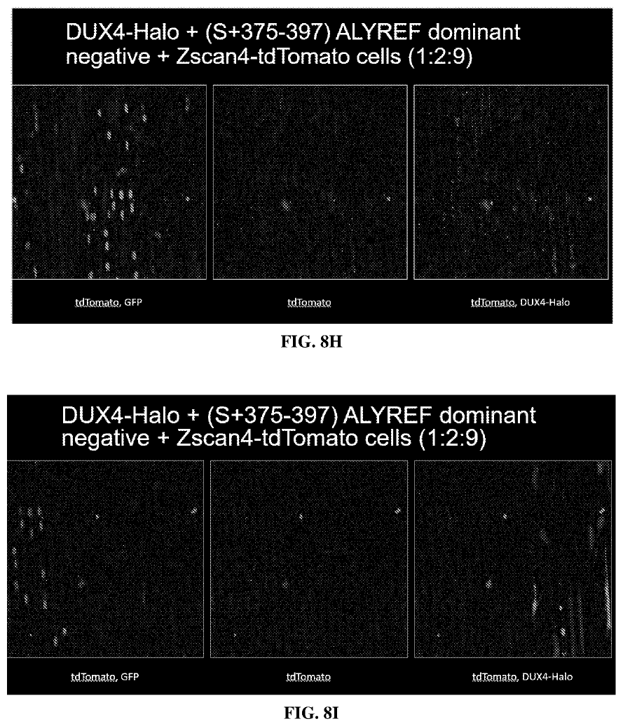

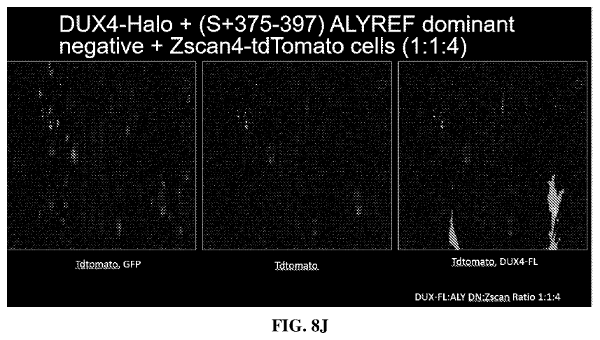

A DUX4 Dominant Negative can compete with full length DUX4 to prevent DUX4-mediated transcription. One such construct is S+375-397 DUX4, which contains HOX1 and HOX2 DNA binding domains, which but only part of the C-terminal domain (FIG. 8A). This construct was identified by Mitsuhashi et al. as a potential inhibitory construct which can bind the DUX4 promoter without being toxic to cells (FIG. 8B).

Muscle cells include cells including multiple nuclei, thus a DUX4 dominant negative may require efficient spreading to multiple myonuclei. Thus, if delivered by AAV, not all myonuclei will generate DUX4 dominant negative RNA, and if the DUX4 dominant negative has nuclear localization activity, then activity of the dominant negative will be limited to only a few nuclei. Addition of nuclear export sequences may enable the DUX4 dominant negative to spread across the myofiber.

### Experimental Design

C2C12 mouse myoblast stable cell lines created and used: Reporter cell line containing Zscan4 promoter driving tdTomato (Zscan4 is a promoter activated by DUX4): cell line that contains tet-inducible DUX4-Halo (Halo is a fluorescent protein that can be visualized upon addition of a small molecule dye); cell line that stably expresses S+375-397 DUX4 dominant negative fused to GFP; and Cell line that stably expresses S+375-397 DUX4 dominant negative fused to GFP (SEQ ID NO: 12) and an ALYREF nuclear export signal (SEQ ID NO: 13).

Cells lines were plated at varying ratios onto a gelatin substrate that facilitates formation of myotubes. The cells were then differentiated into myotubes; each myotube contains a mixture of nuclei from all the different cell lines. Once differentiated (7 days post serum withdrawal), DUX4-Halo is induced with doxycycline-containing media (500 nanograms per milliliter (ng/ml))

Myotubes were imaged 72 hours following doxycycline induction (FIGS. 8C-8J). S+375-397 dominant negative prevents binding of DUX4-Halo to the Zscan promoter, limiting expression of tdTomato, with the ALYREF construct performing more efficiently.

### Conclusions

The ALYREF dominant negative fusion prevents tdTomato expression more effectively than the dominant negative without the ALYREF sequence providing enhanced nuclear-cytoplasmic trafficking which is beneficial to limit DUX4 transcriptional activation (FIGS. 8A-8J).

### Exemplary Sequences

This Table exhibits some exemplary sequences as disclosed by the instant Specification, but is not limiting. This Specification includes a Sequence Listing submitted concurrently herewith as a text file in ASCII format. The Sequence Listing and all of the information contained therein are expressly incorporated herein and constitute part of the instant Specification as filed.

TABLE 2
Exemplary Sequences

| SEQ | | |
|---|---|---|
| ID | | |
| NO: | Sequence* | Description** |
| 1 | APPAQPPSQPQQHYSEGELEEDEDSDDA | NES (ALYREF)- |
| | | NCT_GFP_017; |
| | | NCT_GFP_018; |
| | | NCT_GFP_020 (AA) |
| | | (“Alyref NES + Rev |
| | | NuLS”) |
| 2 | RFEMFRELNEALELKDA | NES (weak P53) - |
| | | NCT_GFP_021 (AA) |
| 3 | PKKKRAVE | NLS (weak) - |
| | | NCT_GFP_017; |
| | | NCT_GFP_020 (AA) |
| | | ″Mutated SV40 NLS″ |
| 4 | MPKKKRAVE | NLS (weak) |
| 5 | PKKKRKV | NLS (strong) - |
| | | NCT_GFP_018; |
| | | NCT_GFP_021 (AA) |
| 6 | RKKRKKK | NoLS- |
| | | NCT_GFP_020 (AA) |
| 7 | MVSKGEELFTGVVPILVELDGDVNGHKFSVSGEGE | eGFP (AA) |
| | GDATYGKLTLKFICTTGKLPVPWPTLVTTLTYGVQ | |
| | CFSRYPDHMKQHDFFKSAMPEGYVQERTIFFKDDG | |
| | NYKTRAEVKFEGDTLVNRIELKGIDFKEDGNILGH | |
| | KLEYNYNSHNVYIMADKOKNGIKVNFKIRHNIEDG | |
| | SVQLADHYQQNTPIGDGPVLLPDNHYLSTQSALSK | |
| | DPNEKRDHMVLLEFVTAAGITLGMDELYKGTAAA | |
| 8 | MIEQDGLHAGSPAAWVERLFGYDWAQQTIGCSDAA | Aminoglycoside 3′ |
| | VFRLSAQGRPVLFVKTDLSGALNELQDEAARLSWL | phosphotransferase |
| | ATTGVPCAAVLDVVTEAGRDWLLLGEVPGQDLLSS | (AA) |
| | HLAPAEKVSIMADAMRRLHTLDPATCPFDHQAKHR | |
| | IERARTRMEAGLVDQDDLDEEHQGLAPAELFARLK | |
| | ARMPDGEDLVVTHGDACLPNIMVENGRFSGFIDCG | |
| | RLGVADRYQDIALATRDIAEELGGEWADRFLVLYG | |
| | IAAPDSQRIAFYRLLDEFF | |
| 9 | MSIQHFRVALIPFFAAFCLPVFAHPETLVKVKDAE | beta-lactamase (AA) |
| | DQLGARVGYIELDLNSGKILESFRPEERFPMMSTF | |
| | KVLLCGAVLSRIDAGQEQLGRRIHYSQNDLVEYSP | |
| | VTEKHLTDGMTVRELCSAAITMSDNTAANLLLTTI | |
| | GGPKELTAFLHNMGDHVTRLDRWEPELNEAIPNDE | |
| | RDTTMPVAMATTERKLLTGELLTLASRQQLIDWME | |
| | ADKVAGPLLRSALPAGWFIADKSGAGERGSRGIIA | |
| | ALGPDGKPSRIVVIYTTGSQATMDERNRQIAEIGA | |
| | SLIKHW | |
| 10 | GACGGATCGGGAGATCTCCCGATCCCCTATGGTGC | /gene = “bla” |
| | ACTCTCAGTACAATCTGCTCTGATGCCGCATAGTT | /locus_tag = “AmpR |
| | AAGCCAGTATCTGCTCCCTGCTTGTGTGTTGGAGG | promoter” |
| | TCGCTGAGTAGTGCGCGAGCAAAATTTAAGCTACA | /label = “AmpR |
| | ACAAGGCAAGGCTTGACCGACAATTGCATGAAGAA | promoter” |
| | TCTGCTTAGGGTTAGGCGTTTTGCGCTGCTTCGCG | /ApEinfo_label = “Amp |
| | ATGTACGGGCCAGATATACGCGTTGACATTGATTA | R promoter” |
| | TTGACTAGTTATTAATAGTAATCAATTACGGGGTC | /ApEinfo_fwdcolor = “pink” |
| | ATTAGTTCATAGCCCATATATGGAGTTCCGCGTTA | /ApEinfo_revcolor = “pink” |
| | CATAACTTACGGTAAATGGCCCGCCTGGCTGACCG | /ApEinfo_graphicform |
| | CCCAACGACCCCCGCCCATTGACGTCAATAATGAC | at = “arrow_data {[0 1 2 |
| | GTATGTTCCCATAGTAACGCCAATAGGGACTTTCC | 0 0 −1][]0} |
| | ATTGACGTCAATGGGTGGAGTATTTACGGTAAACT | width 5 |
| | GCCCACTTGGCAGTACATCAAGTGTATCATATGCC | offset 0” (NT) |
| | AAGTACGCCCCCTATTGACGTCAATGACGGTAAAT | |
| | GGCCCGCCTGGCATTATGCCCAGTACATGACCTTA | |
| | TGGGACTTTCCTACTTGGCAGTACATCTACGTATT | |
| | AGTCATCGCTATTACCATGGTGATGCGGTTTTGGC | |
| | AGTACATCAATGGGGGTGGATAGCGGTTTGACTCA | |
| | CGGGGATTTCCAAGTCTCCACCCCATTGACGTCAA | |
| | TGGGAGTTTGTTTTGGCACCAAAATCAACGGGACT | |
| | TTCCAAAATGTCGTAACAACTCCGCCCCATTGACG | |
| | CAAATGGGGGGTAGGCGTGTACGGTGGGAGGTCTA | |
| | TATAAGCAGAGCTCTCTGGCTAACTAGAGAACCCA | |
| | CTGCTTACTGGCTTATCGAAATTAATACGACTCAC | |
| | TATAGGGAGACCCAAGCTGGCTAGCGTTTAAACTT | |
| | AAGCTTATGCCCAAAAAGAAAAGAGCCGTGGAAGG | |
| | ATCCGAATTCATGGTTTCTAAGGGGGAAGAGCTTT | |
| | TCACGGGAGTCGTCCCGATTCTGGTGGAGTTGGAC | |
| | GGGGATGTTAATGGTCACAAGTTCTCCGTCAGTGG | |
| | TGAGGGGGAGGGGGATGCGACCTACGGCAAACTGA | |
| | CTTTGAAGTTTATATGCACAACAGGCAAACTTCCC | |
| | GTACCCTGGCCTACTTTGGTGACTACGCTTACATA | |
| | CGGGGTCCAGTGCTTCAGTAGATATCCAGATCACA | |
| | TGAAGCAGCACGATTTTTTTAAAAGCGCCATGCCA | |
| | GAGGGCTATGTTCAAGAGAGGACAATTTTCTTCAA | |
| | GGATGACGGCAACTACAAAACTCGGGCTGAGGTCA | |
| | AATTTGAAGGAGATACGCTGGTGAACAGGATAGAA | |
| | CTGAAGGGAATTGACTTCAAGGAGGATGGAAATAT | |
| | TCTCGGGCATAAATTGGAGTATAACTACAATTCTC | |
| | ATAACGTTTACATTATGGCCGATAAACAAAAAAAT | |
| | GGTATAAAGGTTAACTTCAAAATTCGGCATAACAT | |
| | AGAGGACGGGTCAGTGCAGCTCGCAGACCATTACC | |
| | AGCAAAATACGCCGATAGGTGATGGGCCGGTTCTT | |
| | TTGCCTGATAATCACTACCTCAGCACACAGAGTGC | |
| | CCTCAGCAAAGACCCAAACGAAAAACGAGATCATA | |
| | TGGTGCTCCTGGAATTTGTTACAGCGGCAGGAATA | |
| | ACACTGGGAATGGACGAACTTTACAAGGGTACCGC | |
| | GGCCGCAGCTCCTCCGGCACAACCTCCCAGCCAAC | |
| | CTCAGCAGCATTATAGCGAAGGAGAGTTGGAGGAA | |
| | GATGAAGATTCTGATGACGCGCGCAAAAAACGCAA | |
| | AAAAAAATAAGTGACTCGAGTCTAGAGGGCCCGTT | |
| | TAAACCCGCTGATCAGCCTCGACTGTGCCTTCTAG | |
| | TTGCCAGCCATCTGTTGTTTGCCCCTCCCCCGTGC | |
| | CTTCCTTGACCCTGGAAGGTGCCACTCCCACTGTC | |
| | CTTTCCTAATAAAATGAGGAAATTGCATCGCATTG | |
| | TCTGAGTAGGTGTCATTCTATTCTGGGGGGTGGGG | |
| | TGGGGCAGGACAGCAAGGGGGAGGATTGGGAAGAC | |
| | AATAGCAGGCATGCTGGGGATGCGGTGGGCTCTAT | |
| | GGCTTCTGAGGCGGAAAGAACCAGCTGGGGCTCTA | |
| | GGGGGTATCCCCACGCGCCCTGTAGCGGCGCATTA | |
| | AGCGCGGGGGGTGTGGTGGTTACGCGCAGCGTGAC | |
| | CGCTACACTTGCCAGCGCCCTAGCGCCCGCTCCTT | |
| | TCGCTTTCTTCCCTTCCTTTCTCGCCACGTTCGCC | |
| | GGCTTTCCCCGTCAAGCTCTAAATCGGGGGCTCCC | |
| | TTTAGGGTTCCGATTTAGTGCTTTACGGCACCTCG | |
| | ACCCCAAAAAACTTGATTAGGGTGATGGTTCACGT | |
| | AGTGGGCCATCGCCCTGATAGACGGTTTTTCGCCC | |
| | TTTGACGTTGGAGTCCACGTTCTTTAATAGTGGAC | |
| | TCTTGTTCCAAACTGGAACAACACTCAACCCTATC | |
| | TCGGTCTATTCTTTTGATTTATAAGGGATTTTGCC | |
| | GATTTCGGCCTATTGGTTAAAAAATGAGCTGATTT | |
| | AACAAAAATTTAACGCGAATTAATTCTGTGGAATG | |
| | TGTGTCAGTTAGGGTGTGGAAAGTCCCCAGGCTCC | |
| | CCAGCAGGCAGAAGTATGCAAAGCATGCATCTCAA | |
| | TTAGTCAGCAACCAGGTGTGGAAAGTCCCCAGGCT | |
| | CCCCAGCAGGCAGAAGTATGCAAAGCATGCATCTC | |
| | AATTAGTCAGCAACCATAGTCCCGCCCCTAACTCC | |
| | GCCCATCCCGCCCCTAACTCCGCCCAGTTCCGCCC | |
| | ATTCTCCGCCCCATGGCTGACTAATTTTTTTTATT | |
| | TATGCAGAGGCCGAGGCCGCCTCTGCCTCTGAGCT | |
| | ATTCCAGAAGTAGTGAGGAGGCTTTTTTGGAGGCC | |
| | TAGGCTTTTGCAAAAAGCTCCCGGGAGCTTGTATA | |
| | TCCATTTTCGGATCTGATCAAGAGACAGGATGAGG | |
| | ATCGTTTCGCATGATTGAACAAGATGGATTGCACG | |
| | CAGGTTCTCCGGCCGCTTGGGTGGAGAGGCTATTC | |
| | GGCTATGACTGGGCACAACAGACAATCGGCTGCTC | |
| | TGATGCCGCCGTGTTCCGGCTGTCAGCGCAGGGGC | |
| | GCCCGGTTCTTTTTGTCAAGACCGACCTGTCCGGT | |
| | GCCCTGAATGAACTGCAGGACGAGGCAGCGCGGCT | |
| | ATCGTGGCTGGCCACGACGGGCGTTCCTTGCGCAG | |
| | CTGTGCTCGACGTTGTCACTGAAGCGGGAAGGGAC | |
| | TGGCTGCTATTGGGCGAAGTGCCGGGGCAGGATCT | |
| | CCTGTCATCTCACCTTGCTCCTGCCGAGAAAGTAT | |
| | CCATCATGGCTGATGCAATGCGGCGGCTGCATACG | |
| | CTTGATCCGGCTACCTGCCCATTCGACCACCAAGC | |
| | GAAACATCGCATCGAGCGAGCACGTACTCGGATGG | |
| | AAGCCGGTCTTGTCGATCAGGATGATCTGGACGAA | |
| | GAGCATCAGGGGCTCGCGCCAGCCGAACTGTTCGC | |
| | CAGGCTCAAGGCGCGCATGCCCGACGGCGAGGATC | |
| | TCGTCGTGACCCATGGCGATGCCTGCTTGCCGAAT | |
| | ATCATGGTGGAAAATGGCCGCTTTTCTGGATTCAT | |
| | CGACTGTGGCCGGCTGGGTGTGGCGGACCGCTATC | |
| | AGGACATAGCGTTGGCTACCCGTGATATTGCTGAA | |
| | GAGCTTGGCGGCGAATGGGCTGACCGCTTCCTCGT | |
| | GCTTTACGGTATCGCCGCTCCCGATTCGCAGCGCA | |
| | TCGCCTTCTATCGCCTTCTTGACGAGTTCTTCTGA | |
| | GCGGGACTCTGGGGTTCGAAATGACCGACCAAGCG | |
| | ACGCCCAACCTGCCATCACGAGATTTCGATTCCAC | |
| | CGCCGCCTTCTATGAAAGGTTGGGCTTCGGAATCG | |
| | TTTTCCGGGACGCCGGCTGGATGATCCTCCAGCGC | |
| | GGGGATCTCATGCTGGAGTTCTTCGCCCACCCCAA | |
| | CTTGTTTATTGCAGCTTATAATGGTTACAAATAAA | |
| | GCAATAGCATCACAAATTTCACAAATAAAGCATTT | |
| | TTTTCACTGCATTCTAGTTGTGGTTTGTCCAAACT | |
| | CATCAATGTATOTTATCATGTCTGTATACCGTCGA | |
| | CCTCTAGCTAGAGCTTGGCGTAATCATGGTCATAG | |
| | CTGTTTCCTGTGTGAAATTGTTATCCGCTCACAAT | |
| | TCCACACAACATACGAGCCGGAAGCATAAAGTGTA | |
| | AAGCCTGGGGTGCCTAATGAGTGAGCTAACTCACA | |
| | TTAATTGCGTTGCGCTCACTGCCCGCTTTCCAGTC | |
| | GGGAAACCTGTCGTGCCAGCTGCATTAATGAATCG | |
| | GCCAACGCGCGGGGAGAGGCGGTTTGCGTATTGGG | |
| | CGCTCTTCCGCTTCCTCGCTCACTGACTCGCTGCG | |
| | CTCGGTCGTTCGGCTGCGGCGAGCGGTATCAGCTC | |
| | ACTCAAAGGCGGTAATACGGTTATCCACAGAATCA | |
| | GGGGATAACGCAGGAAAGAACATGTGAGCAAAAGG | |
| | CCAGCAAAAGGCCAGGAACCGTAAAAAGGCCGCGT | |
| | TGCTGGCGTTTTTCCATAGGCTCCGCCCCCCTGAC | |
| | GAGCATCACAAAAATCGACGCTCAAGTCAGAGGTG | |
| | GCGAAACCCGACAGGACTATAAAGATACCAGGCGT | |
| | TTCCCCCTGGAAGCTCCCTCGTGCGCTCTCCTGTT | |
| | CCGACCCTGCCGCTTACCGGATACCTGTCCGCCTT | |
| | TCTCCCTTCGGGAAGCGTGGCGCTTTCTCATAGCT | |
| | CACGCTGTAGGTATCTCAGTTCGGTGTAGGTCGTT | |
| | CGCTCCAAGCTGGGCTGTGTGCACGAACCCCCCGT | |
| | TCAGCCCGACCGCTGCGCCTTATCCGGTAACTATC | |
| | GTCTTGAGTCCAACCCGGTAAGACACGACTTATCG | |
| | CCACTGGCAGCAGCCACTGGTAACAGGATTAGCAG | |
| | AGCGAGGTATGTAGGCGGTGCTACAGAGTTCTTGA | |
| | AGTGGTGGCCTAACTACGGCTACACTAGAAGAACA | |
| | GTATTTGGTATCTGCGCTCTGCTGAAGCCAGTTAC | |
| | CTTCGGAAAAAGAGTTGGTAGCTCTTGATCCGGCA | |
| | AACAAACCACCGCTGGTAGCGGTGGTTTTTTTGTT | |
| | TGCAAGCAGCAGATTACGCGCAGAAAAAAAGGATC | |
| | TCAAGAAGATCCTTTGATCTTTTCTACGGGGTCTG | |
| | ACGCTCAGTGGAACGAAAACTCACGTTAAGGGATT | |
| | TTGGTCATGAGATTATCAAAAAGGATCTTCACCTA | |
| | GATCCTTTTAAATTAAAAATGAAGTITTAAATCAA | |
| | TCTAAAGTATATATGAGTAAACTTGGTCTGACAGT | |
| | TACCAATGOTTAATCAGTGAGGCACCTATCTCAGC | |
| | GATCTGTCTATTTCGTTCATCCATAGTTGCCTGAC | |
| | TCCCCGTCGTGTAGATAACTACGATACGGGAGGGC | |
| | TTACCATCTGGCCCCAGTGCTGCAATGATACCGCG | |
| | AGACCCACGCTCACCGGCTCCAGATTTATCAGCAA | |
| | TAAACCAGCCAGCCGGAAGGGCCGAGCGCAGAAGT | |
| | GGTCCTGCAACTTTATCCGCCTCCATCCAGTCTAT | |
| | TAATTGTTGCCGGGAAGCTAGAGTAAGTAGTTCGC | |
| | CAGTTAATAGTTTGCGCAACGTTGTTGCCATTGCT | |
| | ACAGGCATCGTGGTGTCACGCTCGTCGTTTGGTAT | |
| | GGCTTCATTCAGCTCCGGTTCCCAACGATCAAGGC | |
| | GAGTTACATGATCCCCCATGTTGTGCAAAAAAGCG | |
| | GTTAGCTCCTTCGGTCCTCCGATCGTTGTCAGAAG | |
| | TAAGTTGGCCGCAGTGTTATCACTCATGGTTATGG | |
| | CAGCACTGCATAATTCTCTTACTGTCATGCCATCC | |
| | GTAAGATGCTTTTCTGTGACTGGTGAGTACTCAAC | |
| | CAAGTCATTCTGAGAATAGTGTATGCGGCGACCGA | |
| | GTTGCTCTTGCCCGGCGTCAATACGGGATAATACC | |
| | GCGCCACATAGCAGAACTTTAAAAGTGCTCATCAT | |
| | TGGAAAACGTTCTTCGGGGCGAAAACTCTCAAGGA | |
| | TCTTACCGCTGTTGAGATCCAGTTCGATGTAACCC | |
| | ACTCGTGCACCCAACTGATCTTCAGCATCTTTTAC | |
| | TTTCACCAGCGTTTCTGGGTGAGCAAAAACAGGAA | |
| | GGCAAAATGCCGCAAAAAAGGGAATAAGGGCGACA | |
| | CGGAAATGTTGAATACTCATACTCTTCCTTTTTCA | |
| | ATATTATTGAAGCATTTATCAGGGTTATTGTCTCA | |
| | TGAGCGGATACATATTTGAATGTATTTAGAAAAAT | |
| | AAACAAATAGGGGTTCCGCGCACATTTCCCCGAAA | |
| | AGTGCCACCTGACGTC | |
| 11 | MPKKKRAVEGSEFMVSKGEELFTGVVPILVELDGD | NLS-EGFP-NES- |
| | VNGHKFSVSGEGEGDATYGKLTLKFICTTGKLPVP | NuLS |
| | WPTLVTTLTYGVQCFSRYPDHMKQHDFFKSAMPEG | |
| | YVQERTIFFKDDGNYKTRAEVKFEGDTLVNRIELK | |
| | GIDFKEDGNILGHKLEYNYNSHNVYIMADKQKNGI | |
| | KVNFKIRHNIEDGSVQLADHYQQNTPIGDGPVLLP | |
| | DNHYLSTQSALSKDPNEKROHMVLLEFVTAAGITL | |
| | GMDELYKGTAAAAPPAQPPSQPQQHYSEGELEEDE | |
| | DSDDARKKRKKK* | |
| 12 | MALPTPSDSTLPAEARGRGRRRRLVWTPSQSEÅLR | (S+375-397) dominant |
| | ACFERNPYPGIATRERLAQAIGIPEPRVQIWFQNE | negative (EGFP was |
| | RSRQLRQHRRESRPWPGRRGPPEGRRKRTAVTGSQ | removed from the N- |
| | TALLLRAFEKDRFPGIAAREELARETGLPESRIQI | term) |
| | WFQNRRARHPGQGGRAPAQELLASPEFLQQAQPLL | |
| | ETEAPG* | |
| 13 | MALPTPSDSTLPAEARGRGRRRRLVWTPSQSEALR | (S+375-397) ALYREF |
| | ACFERNPYPGIATRERLAQAIGIPEPRVQIWFQNE | dominant negative |
| | RSRQLRQHRRESRPWPGRRGPPEGRRKRTAVTGSQ | (EGFP was removed |
| | TALLLRAFEKDRFPGIAAREELARETGLPESRIQI | from the N-term) |
| | WFQNRRARHPGQGGRAPAQELLASPEFLQQAQPLL | |
| | ETEAPGLEGTRFEAPPAQPPSQPQQHYSEGELEED | |
| | EDSDD* | |
| 14 | DDIVFEDFARLRLKGM | NES - Beta-arrestin-2 |
| 15 | LIAGIIAMIC | NES - β-Dystroglycan |
| 16 | SLPPERMMPMDQTMHPDHTQTVIPYNPSSHESLDQ | NES - Protein kinase C |
| | VGEEKEAMNTRESGKASSSLGLQDFDLL | type |
| 17 | MTLGMIWTIIL | NES - Alpha-actinin-4 |
| 18 | FEALMRMLDNLGYRT | NES - Alpha-adducin |
| 19 | LAQQFEQLSV | NES - argonaute 1 |
| 20 | PFPQDVINKLDKLSVLRLSVS | NES - Aryl |
| | | hydrocarbon receptor |
| 21 | RLGSHTMDFFEMCASLITALAR | NES - AMPK subunit |
| | | alpha-2 |
| 22 | MSHVAVENVLNLDQQFAGLDL | NES - Putative ATP- |
| | | dependent RNA |
| | | helicase an3 |
| 23 | VIQQTLAAIVDAIKLDAI | NES - Ankyrin repeat |
| | | domain-containing |
| | | protein 11 |
| 24 | VGGELLDLLGDLNLS | NES - AP-1 complex |
| | | subunit gamma |
| 25 | VLSAVKVLMKFLELLPKDS | NES - AP-2 complex |
| | | subunit beta |
| 26 | LLERLKELNL | NES - denomatous |
| | | polyposis coli protein |
| 27 | LTKRIDSLPL | NES - denomatous |
| | | polyposis coli protein |
| 28 | ESTPDGFSCSSSLSALSLDEP | NES - denomatous |
| | | polyposis coli protein |
| 29 | EGTPINFSTATSLSDLTIESP | NES - denomatous |
| | | polyposis coll protein |
| 30 | EDTPVCFSRNSSLSSLSIDSE | NES - denomatous |
| | | polyposis coli protein |
| 31 | LWVRLYVLELYCIIL | NES - C → U-editing |
| | | enzyme APOBEC-1 |
| 32 | DVKSLESALKDLKIK | NES - Actin-related |
| | | protein 2/3 complex |
| | | subunit 1B-B |
| 33 | LLRNEVAQLKQLLLAH | NES - Cyclic AMP- |
| | | dependent transcription |
| | | factor ATF-2 |
| 34 | LEELCAARRLSL | NES - Bovine |
| | | herpesvirus infected |
| | | cell protein 27 |
| 35 | ELITFINALKL | NES - Replication |
| | | protein E1 |
| 36 | QLVEELLKIICAFQLDTGL | NES - Breast cancer |
| | | type 1 susceptibility |
| | | protein |
| 37 | DLSDLTFLEVA | NES - Breast cancer |
| | | type 2 susceptibility |
| | | protein |
| 38 | RDLERAMTTLKLWES | NES - Protein C |
| 39 | AGDILALVFGLLFAVTSV | NES - Carbonic |
| | | anhydrase 9 |
| 40 | LDSSLANLVGNLGIGNGT | NES - Clathrin |
| | | assembly lymphoid |
| | | myeloid leukemia |
| | | protein |
| 41 | SSTSGLEQDVAQLNIAEQN | NES - Cancer |
| | | susceptibility candidate |
| | | gene 3 protein |
| 42 | ASSLRDYAASTMTEFLGMFGYDDQNTRDELARKIS | NES - Zinc finger |
| | FEKLHAGSTPEAATSSMLPTSEDTLSK | protein castor homolog |
| 43 | LEMFGPEGAL | NES - Choline- |
| | | phosphate |
| | | cytidylyltransferase A |
| 44 | HLVLIG | NES - Cell division |
| | | control protein 6 |
| | | homolog |
| 45 | LRKLCERLRG | NES - Cell division |
| | | cycle 7-related protein |
| | | kinase |
| 46 | LLDKLLDLNP | NES - Cell division |
| | | cycle 7-related protein |
| | | kinase |
| 47 | EQCERA | NES - Cyclic GMP- |
| | | AMP synthase |
| 48 | VLRMMVGVNI | NES - Calcineurin B |
| | | homologous protein 1 |
| 49 | FVKVLEKVDV | NES - Calcineurin B |
| | | homologous protein 1 |
| 50 | PNCPFLENSLETLRFSISNLSMQ | NES - Baculovimal IAP |
| | | repeat-containing |
| | | protein 2 |
| 51 | LGALWLAL | NES - MHC class II |
| | | transactivator |
| 52 | IHTPVAIIELELGK | NES - COMM domain- |
| | | containing protein 1 |
| 53 | EVKVNQILKTLSEVEES | NES - COMM domain- |
| | | containing protein 1 |
| 54 | DLLGTDQDNLDLANVNLMLELLVQKKKQLEAESHA | NES - E3 ubiquitin- |
| | AQLQILMEFL | protein ligase RFWD2 |
| 55 | LAALNHISAL | NES - Metal-binding |
| | | regulatory protein Cuf1 |
| 56 | LKVIEENVCPKPAQVEPSSPSPMETSGCLPDELCQ | NES - G2/mitotic- |
| | AFSDVLIHVKDVDAD | specific cyclin-B1 |
| 57 | DLCQAFSDVILA | NES - G2/MITOTIC- |
| | | SPECIFIC CYCLIN |
| | | B1 |
| 58 | NSHVAVENALGLDQQFAGLDLN | NES - ATP-dependent |
| | | RNA helicase DDX3X |
| 59 | PFGQALRPLLDSIQI | NES - Desumoylating |
| | | isopeptidase 1 |
| 60 | LSTLDQLRL | NES - diacylglycerol |
| | | kinase zeta |
| 61 | SLEGAVSEISLRD | NES - Protein kinase |
| | | dsk1 |
| 62 | LYPELRRILTI | NES - E1B protein, |
| | | large T-antigen |
| 63 | YLAESSGPARGRGRHPGKGVKSPGEKS | NES - Transcription |
| | | factor E2F1 |
| 64 | TPSAPRPALGRPPVKRRLDLETDHQY | NES - Transcription |
| | | factor E2F1 |
| 65 | MVLTREELVI | NES - E4 34-kDa |
| | | protein |
| 66 | VSQIFPDSVMLAVQEGIDLL | NES - Adenoviral E1A |
| 67 | FDIDEAEEGVKDLKIESDV | NES - Bukaryotic |
| | | translation initiation |
| | | factor 2 subunit 2 |
| 68 | RIDRDVQILNHILDDIEFFITKLQ | NES - Epidermal |
| | | Growth Factor (EGF) |
| | | receptor Pathway |
| | | Substrate 8 |
| 69 | LCNCALEELRL | NES - ETS-related |
| | | transcription factor E1f- |
| | | 3 |
| 70 | LWEFIRDILI | NES - ETS-related |
| | | transcription factor E1f- |
| | | 3 |
| 71 | IHKKFSSIQM | NES - Hox cofactor |
| | | Extradenticle |
| 72 | RLSHLRSEEVHWLHVDMGV | NES - Focal adhesion |
| | | kinase 1 |
| 73 | FLQVRKYSLDLASLILYAYQL | NES - Focal adhesion |
| | | kinase 1 |
| 74 | DLNALLLEVEGPLCKKLSLSKVIDCDS | NES - Fanconi anemia |
| | | group A protein |
| 75 | PQVTVDVLQRMLIFALDALAAG | NES - Fanconi anemia |
| | | group A protein |
| 76 | LADLKVSIENMGLYEDL | NES - Fanconi anemia |
| | | group A protein |
| 77 | LSPGLIKKFQFLMFRLFSEAR | NES - Fanconi anemia |
| | | group A protein |
| 78 | GNEDIISRLQEMVADLELQQDLIVPLGHTPS | NES - Fanconi anemia |
| | | group A protein |
| 79 | LVVLPLELKL | NES - F-box only |
| | | protein 7 |
| 80 | FVEKLQDIQQ | NES - Hypoxia- |
| | | inducible factor 1-alpha |
| | | inhibitor |
| 81 | EVDQLRLERLQIDEQLR | NES - Fragile X mental |
| | | retardation 1 protein |
| 82 | LHGTMRPLSL | NES - Nitrogen |
| | | regulatory protein |
| | | GLN3 |
| 83 | QILPKVLHKRTLGLSAM | NES - Protein X |
| 84 | NVNFVSVGLFRCLPVPCPEDLLVEELVDGLLSL | NES - HTLV-1 basic |
| | | leucine-zipper factor |
| 85 | EAQTCENEEAETVTAMASLSVGVKPA | NES - Histone |
| | | deacetylase 4 (HD4) |
| 86 | EEAETVSAMALLSVGAEQAQAAAAREHSPRPAEEP | NES - Histone |
| | NEQEPAL | deacetylase 5 |
| 87 | LSFDESLALCVIREI | NES - E3 ubiquitin- |
| | | protein ligase MDM2 |
| 88 | LQLPPLERLTLD | NES - Protein Rev |
| 89 | LVDKFMKLDL | NES - MutS protein |
| | | homolog 5 |
| 90 | DTDIQELSEQIHRLLLQPV | NES - Period circadian |
| | | protein homolog 1 |
| 91 | ISPRLDAIKL | NES - Replication |
| | | protein E1 |
| 92 | ARSFEMTEFNQALEEIKGQVVEN | NES - |
| | | Serine/threonine- |
| | | protein kinase R↓O2 |
| 93 | DIELTLRLN | NES - NmrA-like |
| | | family domain- |
| | | containing protein 1 |
| 94 | EELQDLVDQLGFL | NES - Heat shock |
| | | factor protein HSF30 |
| 95 | IEAALSDALAALQI | NES - Heat shock |
| | | protein SSB1 |
| 96 | MATLEKLMKAFESLKSF | NES - Huntingtin |
| 97 | NVAEMVESLQSVLALGHKRNSGVPAFLTPLLRNII | NES - Huntingtin |
| | ISLARLPLYNSYTRVP | |
| 98 | MFQAAERPQEWAMEGPRDGLKKERLLDDRHDSGLD | NES - NF-kappa-B |
| | SMKDEEYEQMVKELQEIRLE | inhibitor alpha |
| 99 | VIDYIRDLQLELNS | NES - DNA-binding |
| | | protein inhibitor ID- 1 |
| 100 | LTTLNTDISILSLQASE | NES - DNA-binding |
| | | protein inhibitor ID-2 |
| 101 | VSKYPLMEEIGFLVLGMRVYHVHSDS | NES - Inositol |
| | | polyphosphate |
| | | multikinase |
| 102 | QEDILDELLGNMVLA | NES - Interferon |
| | | regulatory factor 3 |
| 103 | LQRMLPSLSLT | NES - Interferon |
| | | regulatory factor S |
| 104 | LDRKLLELLW | NES - COP9 |
| | | signalosome complex |
| | | subunit 5 |
| 105 | RCHSLTPNFLQMQLQKCEILQSDSRCKDYLVKIFE | NES - Kelch-like ECH- |
| | ELTLHKPTQ | associated protein 1 |
| 106 | IPYSINMNVFLPDITHLRTGLYKSQRPCVTQ | NES - Krueppel-like |
| | | factor 5 |
| 107 | MDVLPMCSIFQELQIV | NES - Krüppel-like |
| | | factor 6 |
| 108 | MVPLVIKLRL | NES - B-cell specific |
| | | latent nuclear protein |
| 109 | FKPDMNPALREVLEALEDEAYVVND | NES - Protein LTV1 |
| 110 | ALREVLEALEDE | NES - Protein LTV1 |
| 111 | LEKVTNTLSSLKF | NES - Protein LTV1 |
| 112 | LEQELQQLSLEL | NES - Leucine zipper |
| | | putative tumor |
| | | suppressor 2 |
| 113 | LFGDTIAYLLSL | NES - M1 protein |
| 114 | LLYCLMVMYL | NES - M1 protein |
| 115 | QLLQEKLEKLTKLK | NES - Spindle |
| | | assembly checkpoint |
| | | component MAD1 |
| 116 | DKERWEDVKEEMTSALATMRVDYE | NES - MAP Kinase- |
| | | activated protein kinase |
| | | 2 |
| 117 | ALQKKLEELELDE | NES - Dual specificity |
| | | mitogen-activated |
| | | protein kinase kinase 1 |
| | | (MAP kinase kinase 1) |
| 118 | VPEVEALLARLRAL | NES - Protein |
| | | diaphanous homolog 3 |
| 119 | DLVLLSLVL | NES - Menin |
| 120 | QLQQKLLWLL | NES - Menin |
| 121 | LVIAMDQLNL | NES - Microtubule |
| | | protein alp7 |
| 122 | PLPVLGLGGLRISSDS | NES - Mitogen- |
| | | activated protein kinase |
| | | phosphatase 3 |
| 123 | LFDLAMLALD | NES - DNA mismatch |
| | | repair protein Mih1 |
| 124 | LHLVGVNV | NES - Modulator of |
| | | KLF7 activity |
| 125 | ASLTKLFECMTLAYSGKLVS | NES - MLX-interacting |
| | | protein |
| 126 | DIQELSEQIHRLLL | NES - Period circadian |
| | | protein homolog 1 |
| 127 | LEIALRNLNL | NES - Dual specificity |
| | | protein kinase TTK |
| 128 | DEMDSGTMVRAVGDEMGTVRVASTMTDGANTMIEH | NES - |
| | DDTLPSQLGTMVINAEDEEEEGTM | Serine/threonine- |
| | | protein kinase 4 |
| 129 | DGDYEFLKSWTVEDLQKRLLALDPMMEQEIE | NES - |
| | | Serine/threonine- |
| | | protein kinase 4 |
| 130 | PVSKITFVTL | NES - mRNA export |
| | | factor EB2 |
| 131 | LPSPLASLTL | NES - mRNA export |
| | | factor EB2 |
| 132 | LCLSDLSLL | NES - Metal regulatory |
| | | transcription factor 1 |
| 133 | IQDGLLKMLSLVL | NES - Mammalian |
| | | target of rapamycin |
| 134 | ITFIFKSLGL | NES - Mammalian |
| | | target of rapamycin |
| 135 | LEWLRRLSL | NES - Mammalian |
| | | target of rapamycin |
| 136 | VQVVADVLSKLLVYGITDPD | NES - Mammalian |
| | | target of rapamycin |
| 137 | LAPNVAALLFGGNVAVRELADSYEITYNYKMTVPK | NES - Nucleoprotein |
| | SDPNV | |
| 138 | CTLSDSGRISYTVEFSLPTHHTVRLIRVTASPSÅ | NES - N nucleocapsid |
| | | protein |
| 139 | GLPKNVKEKLLSLKTLQSELFEVEKEFQ | NES - Nucleosome |
| | | assembly protein |
| 140 | KKTISPEHVIQALESLGFGSYISEVK | NES - Protein Dτ1 |
| 141 | ILMRMSKMQL | NES - Nuclear export |
| | | protein |
| 142 | MVTRFESLKI | NES - Nuclear export |
| | | protein |
| 143 | YIWALTQTLRIA | NES - Neurogenin-3 |
| 144 | SAIVAAINALTT | NES - Nuclear factor of |
| | | activated T-cells |
| 145 | DÅERNRPLNGGSEPESNSALQEDEREKKDELQTES | NES - Nibrin |
| | WSTKHEIANSDGLQDSSEELPRKLLLTEFRSLVVS | |
| | NHNSTSRNLCVNECG | |
| 146 | KEHKDIDASLDYNSRAQKQEMERAEKDYELFLQEL | NES - 60S ribosomal |
| | EEDAELRQSVNLYKN | export protein NMD3 |
| 147 | LDLPDALLPDLPKL | NES - Nitric oxide- |
| | | associated protein 1 |
| 148 | LLPLAEADKVRLSYLHIMSLACIYT | NES - Neuronal PAS |
| | | domain-containing |
| | | protein 4 |
| 149 | DLCLAVEEVSL | NES - Nucleopbosmin |
| 150 | LKRRLSTLYL | NES - Nuclear factor |
| | | erythroid 2-related |
| | | factor 2 |
| 151 | LRNQLTALRI | NES - NS2 protein |
| 152 | LLLPLMRNLEM | NES - NS2 protein |
| 153 | LVSLIRLKSKL | NES - NS2 protein |
| 154 | DEMTKKFGTLTIHDTEKYASQPELCNN | NES - Marine minute |
| | | virus |
| 155 | LCPDLPELDL | NES - N-truncated |
| | | peroxisome |
| | | proliferator-activated |
| | | receptor gamma co- |
| | | activator 1 alpha |
| 156 | LVDSLQQLRL | NES - Nuclear pore |
| | | complex protein |
| | | Nup214 |
| 157 | LELFVLRLA | NES - NUR-related |
| | | factor 1 |
| 158 | LQRIFYLKL | NES - NUR-related |
| | | factor 1 |
| 159 | LKSLGELGL | NES - Opioid growth |
| | | factor receptor |
| 160 | LSKIATILL | NES - Oligodendrocyte |
| | | transcription factor 1 |
| 161 | LLLAGLPLL | NES - Oligodendrocyte |
| | | transcription factor 1 |
| 162 | HPSTPKRHTVLYISPPPEDLLDNSR | NES - Osmotic |
| | | response element- |
| | | binding protein |
| 163 | VLSQRIGLMDV | NES - Open reading |
| | | frame 45 of Kaposi's |
| | | sarcoma-associated |
| | | herpesvirus 8 |
| 164 | LRHELVEDAVYENPL | NES - ORF9 protein |
| 165 | SLEEELDVLVLDDEGG | NES - Catenin delfa-1 |
| 166 | LTELEISSIFSHCCSLLI | NES - Nucleoprotein |
| | | (N protein) |
| 167 | EMFRELNEALELKD | NES - Cellular tumor |
| | | antigen p53 |
| 168 | NFEILMKLKESLELMELVP | NES - Tumor protein |
| | | p73 |
| 169 | LTLLLDEFENMSV | NES - |
| | | Serine/threonine- |
| | | protein kinase PAK 4 |
| 170 | VVMEFLEGGALTDIV | NES - |
| | | Serinc/threonine- |
| | | protein kinase PAK 4 |
| 171 | LKGFLDRLLV | NES - |
| | | Serine/threonine- |
| | | protein kinase PAK 4 |
| 172 | VYNLVCVALGNLEIREIR | NES - Partner and |
| | | localizer of BRCA2 |
| 173 | ESFDIDDLCSKLKNKAKCS | NES - AP-1-like |
| | | transcription factor |
| 174 | MKPALFNVLCEIKEKTVL | NES - Pre-B-cell |
| | | leukemia transcription |
| | | factor 1 |
| 175 | ILKKVLEALKDLI | NES - Proliferating cell |
| | | nuclear antigen |
| 176 | LLKDLPELALD | NES - Programmed cell |
| | | death protein 4 |
| 177 | VAEMLRDLNLG | NES - Programmed cell |
| | | death protein 4 |
| 178 | LELEALRLSL | NES - Pericentrin |
| 179 | LQDALRRLLGL | NES - Pericentrin |
| 180 | FGETLRAAVTL | NES - Pericentrin |
| 181 | LDEFNELAI | NES - Pericentrin |
| 182 | VIEKLQHELSL | NES - Pericentrin |
| 183 | RVREKQYQDTIGKLQKENNELLEQLEMLQAQLKNS | NES - CCAAT-binding |
| | TLDSPKEVEVNSEVV | factor complex subunit |
| | | Php4 |
| 184 | SLWGEHILALKNLKLDKM | NES - |
| | | Serine/threonine- |
| | | protein kinase PINK1, |
| | | mitochondrial |
| 185 | NELALKLAGLDINKT | NES - CAMP- |
| | | dependent Protein |
| | | Kinase Inhibitor alpha |
| 186 | ELKDFLKELNIQVD | NES - 1- |
| | | phosphatidylinositol- |
| | | 4,5-bisphosphate |
| | | phosphodiesterase |
| | | delta-1 |
| 187 | ISGFLAALPL | NES - Promyelocytic |
| | | leukemia protein |
| | | isoform I |
| 188 | REELWKKLEELKLKKÅLEK | NES - |
| | | Serine/threonine- |
| | | protein phosphatase 2A |
| | | 56 kDa regulatory |
| | | subunit alpha iscform |
| 189 | YEEQLVALFGSSMDLR | NES - Protein kinase, |
| | | CAMP-dependent, |
| | | regulatory subunit type |
| | | Il alpha |
| 190 | IIELLKGLDL | NES - Human |
| | | parainfluenza virus |
| | | type 2 phosphoprotein |
| 191 | LSLNLFALRI | NES - 65 kDa carly |
| | | nonstructural protein |
| 192 | LTLSSLTL | NES - 65 kDa early |
| | | nonstructural protein |
| 193 | VEVLREIQL | NES - Nuclear protein |
| | | UL4 |
| 194 | IMSQFRKLLM | NES - Tegument |
| | | protein UL47 |
| 195 | EVDNLPEDMKRLHLDD | NES - Phosphoprotein |
| 196 | KVAEKLEALSVKEETKED | NES - Ran-specific |
| | | GTPase-activating |
| | | protein |
| 197 | EEILKLLMELVFRLVC | NES - Rap guanine |
| | | nucleotide exchange |
| | | factor 1 |
| 198 | LSEALLQLQF | NES - Transcription |
| | | factor p65 |
| 199 | LEDLVRHMSL | NES - Protein Rev |
| 200 | ALSAQLYSSLSLDS | NES - Rex Protein |
| 201 | EKGNLPELEKLEINGNRLDEDSDALDLLQSKFDDL | NES - Rna1p |
| | EVDDFEE | |
| 202 | GDVFGPELDTLLDSLSLVQGGLSGSGVPSELPQLI | NES - Zinc finger |
| | PV | CCCH domain- |
| | | containing protein 7B |
| 203 | DGFNELIPEDLVTVFDERELELLIGGIAEIDIEDW | NES - E3 ubiquitin- |
| | KKHTDYR | protein ligase RSP5 |
| 204 | QMLSKEVDACVTDLLKELVRFQD | NES - Selenocysteine |
| | | insertion sequence- |
| | | binding protein 2 |
| 205 | QDQFHKMVELTVAAR | NES - Selenocysteine |
| | | insertion sequence- |
| | | binding protein 2 |
| 206 | QYESNENVVLVCSTIVCSFGKQVVEKVE | NES - Protein |
| | | scalloped |
| 207 | LEPDLSEEVSARLRLG | NES - Sentrin-specific |
| | | protease 2 |
| 208 | RRMQEMITRMQAQMQI | NES - Septin A |
| 209 | TGEQELESLVLKLSVLKDF | NES - SH2 domain- |
| | | containing inositol 5′- |
| | | phosphatase 2 |
| 210 | LESLVLKLSVLKDFLSGIQ | NES - SH2 domain- |
| | | containing inositol 5′- |
| | | phosphatase 2 |
| 211 | SRDAARCRRSKETEIFMELSAALPLKTDDVNQLDK | NES - Protein similar |
| | ASVMRITIAFLKIREMLQF | |
| 212 | DMDFLRNLFSQTLSLGSQK | NES - NAD-dependent |
| | | deacetylase sirtuin-2 |
| 213 | LTKMCTIRM | NES - Mothers against |
| | | decapentaplegic |
| | | homolog 1 |
| 214 | GIDLSGLTLQ | NES - Mothers against |
| | | decapentaplegic |
| | | homolog 4 |
| 215 | ELIIGGLDKIDL | NES - B3 ubiquitin- |
| | | protein ligase SMURF1 |
| 216 | SPKAVELTSLSDEDSGKSSQPPSPPSPAPSSFSST | NES - Zinc finger |
| | SÅSSLEAEAFIAFPGLGQLPKQLARLSVAKDPQSR | protein SNAI1 |
| 217 | MEELSQALASSFSVS | NES - Snurportia 1 |
| 218 | ESNGPVKVWGSIKGLTEGLHGFHVHEFGDNTA | NES - Superoxide |
| | | dismutase |
| 219 | LSKTLGKLWRLL | NES - Transcription |
| | | factor SOX-10 |
| 220 | EISKQLGKRWKNLTE | NES - SRY-box |
| | | transcription factor |
| | | SOX14A |
| 221 | EISKQLGRRWKMLTE | NES - SRY-box |
| | | transcription factor |
| | | SOX14B |
| 222 | ELSKTLGKLWRLLN | NES - Transcription |
| | | factor SOX-9 |
| 223 | LEKQINDLQIDK | NES - Spindle pole |
| | | component SPC72 |
| 224 | SASDDLEALGTLSLGTTEE | NES - NCK-interacting |
| | | protein with SH3 |
| | | domain |
| 225 | IYPWMKRVHL | NES - Homeotic |
| | | protein Sex combs |
| | | reduced |
| 226 | LLLKKMYLM | NES - Signal |
| | | transducer and |
| | | activator of |
| | | transcription 1- |
| | | alpha/beta |
| 227 | WDRTESLFQQLIQ | NES - Signal |
| | | transducer and |
| | | activator of |
| | | transcription 1- |
| | | alpha/beta |
| 228 | MEESTNGSLAAEFRHLQLKEQK | NES - Signal |
| | | transducer and |
| | | activator of |
| | | transcription 1- |
| | | alpha/beta |
| 229 | EERIVELFRNLMK | NES - Signal |
| | | transducer and |
| | | activator of |
| | | transcription 3 |
| 230 | LSAEFKHLTLR | NES - Signal |
| | | transducer and |
| | | activator of |
| | | transcription 3 |
| 231 | QLTTLAEKLLGPGV | NES - Signal |
| | | transducer and |
| | | activator of |
| | | transcription 3 |
| 232 | INQMFSVQLSL | NES - Double-stranded |
| | | RNA-binding protein |
| | | Staufen homolog 2 |
| 233 | EATEAQLNNSMAALNVN | NES - Sup35 protcin |
| 234 | VKKQFEELTLGEFLKL | NES - Apoptosis |
| | | inhibitor survivin |
| 235 | YKRIEELLYKISLTT | NES - Protein Tax-1 |
| 236 | NPASAPPPLPPPGQQVIHVTQDLDTDLEÅLFNSVM | NES - |
| | NPKP | NPASAPPPLPPPGQQ |
| | | VIHVTQDLDTDLEA |
| | | LFNSVMNPKP |
| 237 | HVLSSSAGNSAPNSPMAMLHIGSN | NES - Transcription |
| | | Factor EB |
| 238 | MANSANTNTVPKLYRSVIEDVINDVRDIFLDDGVD | NES - Transcription |
| | EQVLM | initiation factor IIA |
| | | subunit 1 |
| 239 | QPDASKADPLPVLENLTLK | NES - Transcription |
| | | factor IIIA |
| 240 | ILRDFFELRLK | NES ~ DNA |
| | | topoisomerase II, alpha |
| | | isozyme |
| 241 | FILEKIDGKIIIE | NES - DNA |
| | | topoisomerase IL, alpha |
| | | isozyme |
| 242 | ETVQDILKEFF | NES - DNA |
| | | topoisomerase II, beta |
| | | isozyme |
| 243 | RISYQPENIQPNRHVANIVEKLREVKLSPEEGQK | NES - Tripartite motif- |
| | | containing protein 5 |
| 244 | QRLQGLPPDRGIIRPGSLDAEIDSLTSMLADLDGG | NES - Thyroid |
| | RSHAP | receptor-interacting |
| | | protein 6 |
| 245 | WMAIAGKIRSDL | NES - Homeotic |
| | | protein Ultrabithorax |
| 246 | CILCQLLLLY | NES - Capsid-binding |
| | | protein UL94 |
| 247 | TTEELELELETLDIN | NES - MGC115246 |
| | | protein |
| 248 | TDPFLAMQVQELTRSMANLTFKQRRDAPPEGPSAK | NES - VEEV Capsid |
| | KPKK | protein |
| 249 | ELDTNFFTLYVAQG | NES - Nucleocapsid |
| | | protein VP1 |
| 250 | LERLFGRLRI | NES - Triplex capsid |
| | | protein VP19C |
| 251 | MDGAIASGVSKFATLSLHD | NES - WD repeat- |
| | | containing protein 20 |
| 252 | IEVEASDLSLSL | NES - DDB1- and |
| | | CUL4-associated factor |
| | | 8 |
| 253 | SSLQELVQQFEALPGDLVG | NES - Amyloid beta |
| | | A4 precursor protein- |
| | | binding family A |
| | | member 3 |
| 254 | EQLERLRKDMGSVAL | NES - GPN-loop |
| | | GTPase 1 |
| 255 | LRLGSQIFI | NES - Tight junction |
| | | protein ZO-2 |
| 256 | LQLVVLRDSK | NES - Tight junction |
| | | protein ZO-3 |
| 257 | LFGPIADIAL | NES - Tight junction |
| | | protein ZO-4 |
| 258 | LEKLANELPDL | NES - Tight junction |
| | | protein ZO-5 |
| 259 | LTMKEVEELELLTQKLM | NES - Zyxin |
| 260 | APPAQPPSQPQQHYSEGELEEDEDSDDA | NES - ALYREF |
| 261 | RFEMFRELNEALELKDA | NES - weak P53 |
| 262 | KRx{10}KKKL | NLS - Nucleoplasmin |
| 263 | PKKKRKV | NLS - SV40, LrgT |
| 264 | PKKNRLRRP | NLS - BRCA1 |
| 265 | PKRPRDRHDGELGGRKRARG | NLS - VirD2-Cterm |
| 266 | PLLKKIKQ | NLS - c-myb |
| 267 | PNKKKRK | NLS - SV40(VP2) |
| 268 | PPQKKIKS | NLS - N-myc |
| 269 | PPRIYPQLPSAPT | NLS - BDV-P |
| 270 | PPRKKRTVV | NLS - NS5A |
| 271 | PPVKRERTS | NLS - RanBP3 |
| 272 | PQPKKKP | NLS - p53 |
| 273 | PQSRKKLR | NLS - Max |
| 274 | PRGRRQPIPKARQP | NLS - HCV |
| 275 | PRPRKIPR | NLS - BDV-P |
| 276 | PRRGPR | NLS - HCV |
| 277 | PRRRK | NLS - SOX9 |
| 278 | PYLNKRKGKP | NLS - Pho4p |
| 279 | PKKARED | NLS - polyoma |
| 280 | PAKRARRGYK | NLS - CPVcapsid |
| 281 | KRx{7,9}PQPKKKP | NLS - p53-NLS1 |
| 282 | KSKKKAQ | NLS - HIV1423 |
| 283 | KTRKHRG | NLS - L29 |
| 284 | KVNSRKRRKEVPGPNGATEED | NLS - CTP |
| 285 | KVTKRKHDNEGSGSKRPK | NLS - hum-Ku70 |
| 286 | K[RK]{3,5)x{11,18}[RK]Kx{2,3}K | NLS - |
| 287 | LEKKVKKKFDWCA | NLS - prot.Hsci |
| 288 | LKDVRKRKLGPGH | NLS - DNAseBBV |
| 289 | LKRKLQR | NLS - Pax-QNR |
| 290 | LKRPRSPSS | NLS - EBNA1 |
| 291 | MAPSAKATAAKKAVVKGTNGKKALKVRTSATFRLP | NLS - L25 |
| | KTLKLAR | |
| 292 | MNKIPIKDLLNPG | NLS - Mat-alpha |
| 293 | MPKTRRRPRRSQRKRPPT | NLS - Rex |
| 294 | MPTEERVRKRKESNRESARRSRYRKAAHLK | NLS - opaque2 |
| 295 | NQSSNFGPMKGGNFGGRSSGPYGGGGQYFAKPRNQ | NLS - M9 |
| | GGY | |
| 296 | PÅAKRVKLD | NLS - IscMyc |
| 297 | QRKRQK | NLS - NF-KB |
| 298 | RAIKRRPGLDFDDDGEGNSKFLR | NLS - hARNT |
| 299 | REKKEKEQKEKCA | NLS - prot.Hsc9 |
| 300 | RRPSx{22}RRKRQ | NLS - PK-A |
| 301 | RRRx{11}KRRK | NLS - CBP80 |
| 302 | RRSMKRK | NLS - hVDR |
| 303 | RVHPYQR | NLS - QKI-5 |
| 304 | SÅNKVTKNKSNSSPYLNKRGKPGPDS | NLS - Pho4 |
| 305 | SDKKVRSRLIECA | NLS - Ta.alpha |
| 306 | SKRVAKRKL | NLS - c-erb-A |
| 307 | SxGTKRSYxxM | NLS - FluA |
| 308 | TEKK[QG]KSILYDCÅ | NLS - prot.Hsc3 |
| 309 | TKRSxxxM | NLS - influenzaNP |
| 310 | VNEAFETLKRC | NLS - MyoD |
| 311 | VSRKRPR | NLS - polyoma |
| 312 | VKRPCKRSFIRFI | NLS - DNAseEBV |
| 313 | YLTQETNKVETYKEQPLKTPGKKKKGKP | NLS - PTHrP |
| 314 | YNNQSSNFGPMKGGN | NLS - M9 |
| 315 | [KAR]TPIQKHWRPTVLTEGPPVKIRIETGEWE | NLS - ASVintegrase |
| | [KA] | |
| 316 | RRMKWKK | NLS - PDX-1 |
| 317 | RRKGKEK | NLS - Hunt.Dis.pro |
| 318 | RGRRRRQR | NLS - Amida |
| 319 | RIRKKLR | NLS - p54 |
| 320 | RKCLQAGMNLEARKTKK | NLS - hGlu.cort. |
| 321 | RKEWLTNFMEDRRQRKL | NLS - hDNAtopoII |
| 322 | RKKRKx{9}KAKKSK | NLS - N1N2 |
| 323 | RKKRRQRRR | NLS - HIV-1Tat |
| 324 | RKRAFHGDDPFGEGPPDKK | NLS - ICP-8 |
| 325 | RKRIREDRKATTAQKVQQMKQRLNENERKRKR | NLS - TCPTP |
| 326 | RKRKK | NLS - ystDNApolalpha |
| 327 | RKRKKMPASQRSKRRKT | NLS - hBLM |
| 328 | RKRRR | NLS - Amida |
| 329 | RLKKLKCSKx{19}KTKR | NLS - GAL4 |
| 330 | RPRRK | NLS - SRY |
| 331 | RQARRNRRRRWR | NLS - HIV-1Rev |
| 332 | RRERNKMAAAKCRNRRR | NLS - cFOS |
| 333 | RRERx{4}RPRKIPR | NLS - BDV-P |
| 334 | [KR]{4}x{20,24}K{1,4}xK | NLS - |
| 335 | KKKRERLD | NLS - RCP |
| 336 | KRKRRP | NLS - BRCA1 |
| 337 | IKYFKKFPKD | NLS - yeast, SK13 |
| 338 | HRKYEAPRHx{6}PRKR | NLS - L3 |
| 339 | KRKx{11}KKKSKK | NLS - hpoly(ADP)poly |
| 340 | HRIEEKRKRTYETFKSI | NLS - NF-kB |
| 341 | KRKx{22}KELQKQITK | NLS - HIV-1 |
| 342 | KRMRNRIAASKCRKRKL | NLS - cJUN |
| 343 | KRPAATKKAGQAKKKK | NLS - Nucloplasmin |
| 344 | EEDGPQKKKRRL | NLS - polyomaVP2 |
| 345 | KRPACTLKPECVQQLLVCSQEAKK | NLS - HCDA |
| 346 | KRPAEDMEEEQAFKRSR | NLS - humKprotein |
| 347 | KRPMNAFIVWSRDQRRK | NLS - SRY |
| 348 | KRPMNAFMVWAQAARRK | NLS - SOX9 |
| 349 | KRKKEMANKSAPEAKKKK | NLS - Nucleolin |
| 350 | APKRKSGVSKC | NLS - polyomaVP1 |
| 351 | KRAAEDDEDDDVDTKKQK | NLS - hProTalpha |
| 352 | KKKRRSREK | NLS - TCF-1 |
| 353 | KKKKKEEEGEGKKK | NLS - act/inh, betaA |
| 354 | KKKYKLK | NLS - HIV1422 |
| 335 | KKQTTLAFKPIKKGKKR | NLS - hDNAtopoII |
| 356 | CKRKTTNADRRKA | NLS - MyoD |
| 357 | KKEKKKSKK | NLS - dyskerin |
| 358 | KHRKHPG | NLS - L29 |
| 359 | KRx{9}KTKK | NLS - THOV |
| 360 | CYGSKNTGAKKRKIDDA | NLS - |
| 361 | KKSKKGRQEALERLKKA | NLS - hDNApolalpha |
| 362 | KDCVINKHHRNRCQYCRLQR | NLS - TR2 |
| 363 | KAKRQR | NLS - v-Rel |
| 364 | KKx{15}KKRK | NLS - DNAhelicaseQ1 |
| 365 | EYLSRKGKLEL | NLS - VirD2-Nterm |
| 366 | KRQRx{20}KKSKK | NLS - Mitosin |
| 367 | KRSAEGGNPPKPLKKLR | NLS - p110RB1 |
| 368 | APTKRKGS | NLS - SV400VP1 |
| 369 | KKKKRKREK | NLS - LBF-1 |
| 370 | KRPRP | NLS - adenovE1a |
| 371 | GRKRKKRT | NLS - Tst1/Oct6 |
| 372 | HKKKKIRTSPTFTTPKTLRLRRQPKYPRKSAPRRN | NLS - BIB |
| | KLDHY | |
| 373 | GGGx{3}KNRRx{6}RGGRN | NLS - Nab2 |
| 374 | GKKRSKA | NLS - H2B |
| 375 | GKKKYKLKH | NLS - HIV-1 |
| 376 | [DE]K[NIF]RR[DEK][STMNQ] | NLS - |
| 377 | [DE]KR[MQN]R[MQN]R | NLS - |
| 378 | [DE]KK[PL][GL]K[GL] | NLS - |
| 379 | R[RK]x{4,6}[RK][RK]x[RK]x{1,3}[RK] | NLS - |
| | [RK][PLQ] | |
| 380 | R[RK]{2,4}[PL][RK][MNQ]R | NLS - |
| 381 | \|R[RK]{2,4}x{15,19}[RK]{2,4}[QLM]K | NLS - |
| 382 | R[RK]{3,)?[DE]K | NLS - |
| 383 | [DE]KxRRK[MNQ] | NLS - |
| 384 | [DE]RKRR[DEPLQ] | NLS - |
| 385 | [DE]RxKKKK | NLS - |
| 386 | [DE][RK]{2,4}[GA]R[PL][GA] | NLS - |
| 387 | R[PL]xx[KR]{2,}?xx[KR]V | NLS - |
| 388 | [DE][KR]RR[KR][FYW] | NLS - |
| 389 | R[QMPL]RR[DE]R | NLS - |
| 390 | R[RK]K[RK]KR | NLS - |
| 391 | R[RK]x[KR]x[RK]{2 ,}?[DE] | NLS - |
| 392 | R[STCMNQ]R[STCMNQ]KR | NLS - |
| 393 | [DE]R{2,4}xRK[PL] | NLS - |
| 394 | RxKKKK[DE] | NLS - |
| 395 | R{2,}?PR{3,)? | NLS - |
| 396 | T[PLV]KRC | NLS - |
| 397 | R{2,3}xK{2,3}R[ST] | NLS - |
| 398 | D[KR]x{0,1}[QL][RK]{2,3}R | NLS - |
| 399 | R{2,3}K{3,4}[PLRKE] | NLS - |
| 400 | Rx{2,3}RRRRRR | NLS - |
| 401 | Rx{2,3}Hx{3,5}RRRR | NLS - |
| 402 | DR[MN]KKKKE | NLS - |
| 403 | WKQ[KR]RKF | NLS - |
| 404 | Rx[KR][KR][KR]xxRKKR | NLS - |
| 405 | RX[KR][KR]K[PLQM]R | NLS - |
| 406 | RxR{2,}?[QL]x[ST]R | NLS - |
| 407 | RxRxRxRxRxRxK | NLS - |
| 408 | RxRxRxRxRxR | NLS - |
| 409 | RxRSRSx{0,1}RxR | NLS - |
| 410 | DK[QL]KK[QL] | NLS - |
| 411 | RxRRx{4,6}RKK | NLS - |
| 412 | R{2,)}[QMN]R{3,)? | NLS - |
| 413 | [TS][RK]KK[VLI]R[PL] | NLS - |
| 414 | [QL]xKRxKxKK | NLS - |
| 415 | [QL]K{2,4}x{8,12}[RK][QL][RK][QL]KR | NLS - |
| 416 | [PVLI][RK][RK][RK][RK][RK][QMN]K | NLS - |
| 417 | [PL]xxKR[IV]K[PL][DE] | NLS - |
| 418 | [PL][RK]{2,3}K[PLI][RK]x[PLI]xK | NLS - |
| 419 | [PL][RK][RK][KR][GAPL][RK][STQM] | NLS - |
| 420 | [PL][RK][RK][DEP]R[RK][FYW] | NLS - |
| 421 | [PL][PL]x[KR]R[DE][KR][QST] | NLS - |
| 422 | [PL][KR]{5,7}[PL] | NLS - |
| 423 | [PL]R[DE]K[DE]R | NLS - |
| 424 | [PL]RKRK[PL] | NLS - |
| 425 | [RK]H[RK]xxx[RK]{2,4}xR | NLS - |
| 426 | [RK]K{2,4}x[RK][QL][RK][PL] | NLS - |
| 427 | [RK]R[MS]KxK[KR] | NLS - |
| 428 | [ST]Gx{1,3}G{3,}?x{1,2}G{3,}?[ST] | NLS - |
| 429 | [STQM]RRRK[STQM] | NLS - |
| 430 | [STQM]RKRR[STQM] | NLS - |
| 431 | [STQM]RKRK[STQM] | NLS - |
| 432 | [RK]{4,}?[QMNPL][RK]x{3,4}[RK]{2} | NLS - |
| 433 | [RK]{3,}?x(8,16)[RK]{4,}? | NLS - |
| 434 | [RK]{3,}?x[RK]x[RK]x{4,9}[RK]{3,}? | NLS - |
| 435 | [RK]{2,4}x{2,4}[QLM][RK]x{2,3}[RK]KR | NLS - |
| 436 | [RK]{2,4}x{1,2}[RK]x{0,2}[RK]x{3,5} | NLS - |
| | [RK]x{0,2}[AK][RK]{2,4}[PL] | |
| 437 | [RK]x[RK]x[KR]x{4,6}RKK | NLS - |
| 438 | [RK][PLIV][KR][RK]{2,4}[PLVI]R | NLS - |
| 439 | [PL]KxxKRR | NLS - |
| 440 | [PLV]RK[ST]R[DE]K | NLS - |
| 441 | [YFW]RRRR[PL] | NLS - |
| 442 | [KR][DE][KR][DE]xx[KR]{4,)? | NLS - |
| 443 | [KR]XXKNKX{6,8}K[KR] | NLS - |
| 444 | [KR]KRKK | NLS - |
| 445 | [KR]G{2,}?xxG{3,}?[RK] | NLS - |
| 446 | [QMN]R[RK]xXx[RK][RK] | NLS - |
| 447 | [GA][KR]KRX[KR][GA] | NLS - |
| 448 | [GA]Rx[RK]x[RK][R]x[QM] | NLS - |
| 449 | [GA]KxKKK[MNQ] | NLS - |
| 450 | [GAPLV]RKRKKR | NLS - |
| 451 | [ED]R{4,}?[ED] | NLS - |
| 452 | [DE][ST][PL]KR[STC] | NLS - |
| 453 | [KR][KR][KR][KR][KR][KR][KR] | NLS - |
| 454 | [KR][KR][QMN]R[RK][QMN]R | NLS - |
| 455 | [KR][KR]x[KR][KR][KR]x[KR][KR] | NLS - |
| 456 | [PLV]K[RK]x[RK][RK][RK][PL] | NLS - |
| 457 | [PLV]K[RK]x[QMN][RK]R | NLS - |
| 458 | [PLQ][KR]x{3,4}KKRK | NLS - |
| 459 | [PLQ]K[RK]x{1,2}[RK]x{3,6}[RK][RK]x | NLS - |
| | {1,2}[RK]x{1,2)[RK][RK] | |
| 460 | [PLQMNKR]K[KR][KR]RxK[PLQMNKR] | NLS - |
| 461 | [PLQMKR]R[KR][QM][KR]RxK | NLS - |
| 462 | [MI]VWSRD[HEQ]RRK | NLS - |
| 463 | [LF][STK][VIQM][KR]R[QMVI][STK]L | NLS - |
| 464 | [KR]{2}x{0,1}[KR]{2,4}x{25,34}K{2,4} | NLS . |
| | x{1,2}K | |
| 465 | [KR]{2,}?[PL]x{1,4][KR]{2,}?x{1,5}K | NLS - |
| | {3,}? | |
| 466 | [KR]{2,3}xxKR[KR][QLM] | NLS - |
| 467 | [DE][RK]{3,}?x[KR]{2,}?[PL] | NLS - |
| 468 | KKRKRT | NLS - |
| 469 | KRKx{10,14}[KR]{3,}?x[KR]K | NLS - |
| 470 | PK[KR][KRP][RAK][KT][VSE] | NLS - |
| 471 | PKRPx{5,8}Lx{2,4}RxKxK | NLS - |
| 472 | KRKx{2,4}DRRK | NLS - |
| 473 | PKKKxRK | NLS - |
| 474 | KRKX{5,10}KK[PL]K | NLS - |
| 475 | N[QR]RQ[RK][EG]KR[IVLS] | NLS - |
| 476 | LKKIKQ | NLS - |
| 473 | KR[GPL]R[GPL]R[GLP]RK | NLS - |
| 478 | KR[MNSQ]R[MNSQ]R | NLS - |
| 479 | KRKx{0,8}KR[PL]K | NLS - |
| 480 | KRGRGRPRK | NLS - |
| 481 | KKx{1,7}K[PL][PLIV]KK | NLS - |
| 482 | RER[MNQ]Kx{4,8}R[MNQ]RR | NLS - |
| 483 | KKRKR[KR] | NLS - |
| 484 | KKRKR[ST] | NLS - |
| 485 | Q[RK][HRK][RK]xRR | NLS - |
| 486 | KKRRK | NLS - |
| 487 | QNRRxKx[RK][RK][DQE] | NLS - |
| 488 | Px[PQLVMN][KR]{2,3}xKQ | NLS - |
| 489 | KKRRxK | NLS - |
| 490 | KK[MNQSTC]R[MNQSTC]K[MNQSTC] | NLS - |
| 491 | KKxRx{3,5}R[PVL]K | NLS - |
| 492 | K{3,4}R{2,3} | NLS - |
| 493 | K{1,}?R{2,}?[QM]R{2,} | NLS - |
| 494 | KxxKxKxKxxxxxRKK | NLS - |
| 495 | K[GA]K[AG]KK[AG] | NLS - |
| 496 | KR[RK][RK]x{2,4}[RK]x{0,2}Rx{3,5}[RK] | NLS - |
| | x{0,2}[RK]x{0,2}[RK][RK]K | |
| 497 | KR[ST]RxxR{2,4}[QL]K | NLS - |
| 498 | KRxRxRx{2,6]RKRK | NLS - |
| 499 | KRxRxxRRLK | NLS - |
| 500 | KR{3,}?[LVI] | NLS - |
| 501 | KR{2,4}x{3,6}[RK]{2,4}x{0,2}KR | NLS - |
| 502 | KKRR[DE]K | NLS - |
| 503 | KRx[DE][KR][KR]xK | NLS - |
| 504 | KRxxKKxK[DE] | NLS - |
| 505 | K[IVQM]RR[VI][STK]L | NLS - |
| 506 | K[KR][KR]RR[KR] | NLS - |
| 507 | K[KR][QMN][RK]R[QMN]R | NLS - |
| 508 | Kx[PLV][RK][RK]RK | NLS ~ |
| 509 | KxKxKxxxxxRKK | NLS - |
| 510 | KxKRQR | NLS - |
| 511 | KR[PLV][GA]KRK[PL] | NLS - |
| 512 | K[RK]{2,}?[QL]x{3,8}R{3} | NLS ~ |
| 513 | K[RK]{2,4}[ST]H | NLS - |
| 514 | K[PL]K{3,}?xKK | NLS - |
| 515 | K[PL]K{2,3}x{1,3}[RK]{2,4}x{6,9}K[KR] | NLS - |
| 516 | K[PLMN]RRK[MNQ] | NLS - |
| 517 | K[MNQ]RR[PLVI]K[PL] | NLS - |
| 518 | KRx{1,3}Hx{3,5}R[LQ]RR | NLS - |
| 519 | R[PL]xGx[KR][KR]xK | NLS - |
| 520 | RRxRxKxKQ | NLS - |
| 521 | RRxR[PVL]RK | NLS - |
| 522 | RRxRRRRR | NLS - |
| 523 | RRxKRxK[PLV] | NLS - |
| 524 | RR[TS]x[QK][KR][KN] | NLS - |
| 525 | RR[TS]x[QK][KR][KNS] | NLS - |
| 526 | RR[PLQMN]xRRRR | NLS - |
| 527 | RR[PLIV]RKxK | NLS - |
| 528 | G{2,4}[RK]x{1,3}G{3} | NLS - |
| 529 | RRR{3,5}T | NLS - |
| 530 | RRxRxRKQ | NLS - |
| 531 | RRxxKRK | NLS - |
| 532 | RRx{0,1}RRRRR | NLS - |
| 533 | R[MNQ]x{4,8}R[MNQ]RR | NLS - |
| 534 | R[MNQ]RRRRxR | NLS - |
| 535 | R[KR]{3,4}K[DE] | NLS - |
| 536 | R[KR][RK]x{0,2}[RK]x{0,2}[RK]x{3,5} | NLS - |
| | [RK]x{0,2}[AK][RK][RK][RK][PMQL] | |
| 537 | R[KR]RRRRxR | NLS - |
| 538 | R[IVLP][IVLP]KRR | NLS - |
| 539 | R[GVLIP]RRRRxR | NLS - |
| 540 | R[GA]x{0,2}[GA]R[GA]x[GA]R[GA] | NLS - |
| 541 | RIGA][IVLP]KRR | NLS - |
| 542 | GR[RK]{2,4}xx[RK][QL] | NLS - |
| 543 | RRR[PL]RK | NLS - |
| 544 | RRR[LP]xxR[PLQ] | NLS - |
| 545 | RRRRRxRR | NLS - |
| 546 | RKR{3,5}[ST] | NLS - |
| 547 | RKRx{12,16}RRKK | NLS - |
| 548 | RKR[PLQMN]R[PLQMN]R | NLS - |
| 549 | RKRKR[KR] | NLS - |
| 550 | KKKKKx{3,6}KK | NLS - |
| 551 | KKKKR[KR] | NLS - |
| 552 | RKKRRxR | NLS - |
| 553 | KKKR[KR][VPL] | NLS - |
| 554 | RKKRKR | NLS - |
| 555 | KKPx{6,9}Kx{1,3}RK | NLS - |
| 556 | RK[IVE]W[ML][TQR]N[HF] | NLS - |
| 557 | RK[PL][PLV]KK[RKH] | NLS - |
| 558 | RK[RK][QML][RK]xR | NLS - |
| 559 | RRRRRRx{0,2}R | NLS - |
| 560 | RRRRRR | NLS - |
| 561 | RRRK[STC]K | NLS - |
| 562 | RRRKKR | NLS - |
| 563 | RRKx{5,7}RRR | NLS - |
| 564 | RRKX{3,5}R[DE]R{3,}?[PLV] | NLS - |
| 565 | RRER[MNQ]Kx{4,8}R[MNQ]RRR | NLS - |
| 566 | KHLKGR | NLS - |
| 567 | RKx{7,12}RK[STMNQ]KK | NLS - |
| 568 | RK]{2,4}[PL][RK]x{7,11}[RK][QL]KH | NLS - |
| 569 | RH[RK]Hx{2,4}[RK]{2,4)}[PL]R | NLS - |
| 570 | PKKKRAVE | NLS - weak sv40 |

*Unless otherwise specified, nucleic acid sequences are described 5′ to 3′ and amino acid sequences are described N-terminus to C-terminus
**‘NT’ denotes a nucleic acid sequence; ‘AA’ denotes an amino acid sequence.

### Other Embodiments

Embodiment 1. A method comprising administering an isolated nucleic acid to a multinucleate cell, wherein the isolated nucleic acid comprises a sequence encoding a fusion protein, the fusion protein comprising, a protein of interest fused to at least the following migration signals: (a) at least one nuclear export signal (NES); and (b) at least one nuclear localization signal (NLS) and/or at least one nucleolar localization signal (NoLS), optionally, wherein the isolated nucleic acid encodes an amino acid sequence comprising a sequence with at least 70% identity to SEQ ID NO: 13.

Embodiment 2. The method of embodiment 1, wherein the fusion protein further comprises at least one additional migration signal, wherein the additional migration signal may be identical or distinct from the migration signals used in embodiment 1.

Embodiment 3. The method of any one of embodiments 1-2, wherein the fusion protein further comprises at least two additional migration signals, wherein the additional migration signals may be identical or distinct from the migration signals used in any one of embodiments 1-2.

Embodiment 4. The method of any one of embodiments 1-3, wherein the isolated nucleic acid encode a fusion protein wherein at least one of the migration signals is positioned at the C-terminus of the protein of interest.

Embodiment 5. The method of any one of embodiments 1-3, wherein the isolated nucleic acid encodes a fusion protein wherein at least one of the migration signals is positioned at the N-terminus of the protein of interest.

Embodiment 6. The method of any one of embodiments 1-5, wherein the protein of interest is a therapeutic protein.

Embodiment 7. The method of any one of embodiments 1-5, wherein the protein of interest is a nuclear protein.

Embodiment 8. The method of any one of embodiments 1-5, wherein the protein of interest is at least uric of the following: a transcriptional factor, a transcriptional repressor, an RNA binding protein, a DNA modifying protein, a DNA editing protein, and a Cas protein.

Embodiment 9. The method of any one of embodiments 1-5, wherein the protein of interest is DUX4, or variant thereof.

Embodiment 10. The method of any one of embodiments 1-5, wherein the protein of interest carries RNA.

Embodiment 11. The method of any one of embodiments 1-5, wherein the protein of interest be used to treat Facioscapulohumeral dystrophy.

Embodiment 12. The method of any one of embodiments 1-11, wherein the isolated nucleic acid is flanked by adeno-associated virus (AAV) inverted terminal repeats (ITRs).

Embodiment 13. The method of any one of embodiments 1-12, wherein the multinucleate cell is a skeletal muscle cell.

Embodiment 14. The method of any one of embodiments 1-12, wherein the multinucleate cell is a liver cell.

Embodiment 15. The method of any one of embodiments 1-14, wherein the nucleic acid is administered to a subject.

Embodiment 16. The method of any one of embodiments 1-15, wherein the subject has a disorder.

Embodiment 17. The method of embodiment 16, wherein the protein of interest treats the disorder.

Embodiment 18. The method of any one of embodiments 16-17, wherein the disorder is Facioscapulohumeral dystrophy.

Embodiment 19. The method of any one of embodiments 16-18, wherein the protein of interest treats Facioscapulohumeral dystrophy.

Embodiment 20. The method of any one of embodiments 15-19, wherein the subject is mammalian.

Embodiment 21. The method of any one of embodiments 15-20, wherein the subject is human.

Embodiment 22. A fusion protein comprising: (a) a protein of interest; and (b) at least the following migration signals: (i) a nuclear export signal (NES): (ii) a nuclear localization signal (NLS); and (iii) a nucleolar localization signal (NoLS), optionally, wherein the fusion protein comprises a sequence with at least 70% identity to SEQ ID NO: 13.

Embodiment 23. The fusion protein of embodiment 22, further comprising at least one additional migration signal, wherein the additional migration signal may be identical or distinct from the migration signals used in embodiment 22.

Embodiment 24. The fusion protein of any one of embodiments 22-23, further comprising at least two additional migration signals, wherein the additional migration signals may be identical or distinct from the migration signals used in any one of embodiments 22-23.

Embodiment 25. The fusion protein of any one of embodiments 22-24, wherein the at least one of the migration signals is positioned at the C-terminus of the protein of interest.

Embodiment 26. The fusion protein of any one of embodiments 22-24, wherein at least one of the migration signals is positioned at the N-terminus of the protein of interest. .embodiment

Embodiment 27. The fusion protein of any one of embodiments 22-26, wherein at least one of the migration signals is linked to the protein of interest via a linker.

Embodiment 28. The fusion protein of any one of embodiments 22-27, wherein at least one of the migration signals comprises a sequence with at least 95% identity to SEQ ID NO: 1-6 or 14-570.

Embodiment 29. The fusion protein of any one of embodiments 22-28, wherein at least one of the migration signals comprises a sequence with at least 95% identity to SEQ ID NO: 3.

Embodiment 30. The fusion protein of any one of embodiments 22-29, wherein at least one of the migration signals comprises a sequence of SEQ ID NO: 1.

Embodiment 31. The fusion protein of any one of embodiments 22-30, wherein at least one of the migration signals comprises a sequence of SEQ ID NO: 3.

Embodiment 32. The fusion protein of any one of embodiments 22-31, wherein at least one of the migration signals comprises a sequence of SEQ ID NO: 1 and at least one of the migration signals comprises a sequence of SEQ ID NO: 3.

Embodiment 33. The fusion protein of the method of any one of embodiments 1-21, or the fusion protein of any one of embodiments 22-31, wherein at least one of the migration signals is linked to at least one other migration signal via a linker.

Embodiment 34. An isolated nucleic acid comprising a nucleic acid sequence encoding the fusion protein of the method of any one of embodiments 1-21, or the fusion protein of any one of embodiments 22-31.

Embodiment 35. The isolated nucleic acid of embodiment 34, further comprising a promoter operably linked to the sequence encoding the fusion protein.

Embodiment 36. The isolated nucleic acid of embodiment 35, wherein the promoter is a constitutive promoter, an inducible promoter, or a tissue specific promoter.

Embodiment 37. The isolated nucleic acid of any one of embodiments 35-36, wherein the promoter is a tissue specific promoter.

Embodiment 38. The isolated nucleic acid of embodiment 37, wherein the tissue specific promoter is specific to skeletal muscle.

Embodiment 39. The isolated nucleic acid of embodiment 37, wherein the tissue specific promoter is specific to liver tissue.

Embodiment 40. The isolated nucleic acid of any one of embodiments 34-39, further comprising at least one additional regulatory sequence.

Embodiment 41. A recombinant adeno-associated virus (rAAV), comprising: (a) the isolated nucleic acid of any one of embodiments 34-40 flanked by adeno-associated virus (AAV) inverted terminal repeats (ITRs); and (b) an AAV capsid protein.

Embodiment 42. The rAAV of embodiment 41, wherein the AAV capsid protein exhibits a tropism for skeletal muscle.

Embodiment 43. The rAAV of embodiment 41, wherein the AAV capsid protein exhibits a tropism for liver tissue.

Embodiment 44. The rAAV of any one of embodiments 41-42, wherein the AAV capsid protein serotype is selected from: AAV1 and AAV6.

Embodiment 45. The rAAV of any one of embodiments 44, wherein the AAV capsid protein serotype is AAV6.

Embodiment 46. The rAAV of any one of embodiments 41-43, wherein the AAV capsid protein serotype is selected from: AAV7; AAV8; and AAV9.

Embodiment 47. A composition comprising the isolated nucleic acid of any one of embodiments 34-40, or the rAAV of any one of embodiments 41-46, and a pharmaceutically acceptable excipient.

Embodiments 48. The composition of embodiment 47, wherein the composition is formulated for intramuscular administration, intrathecal administration, intravenous administration, retrograde intravenous administration, intravascular administration, systemic administration, intra-arterial administration, intraportal administration, intratracheal administration, or direct injection.

Embodiment 49. A method of delivering a fusion protein to multinucleate cells, comprising, administering the fusion protein of any one of embodiments 22-32, the isolated nucleic acid of any one of embodiments 34-40, the rAAV of any one of embodiments 41-46, or the composition of any one of embodiments 47-48 to a subject.

Embodiment 50. The fusion protein of any one of embodiments 22-32, the isolated nucleic acid of any one of embodiments 34-40, the rAAV of any one of embodiments 41-46, or the composition of any one of embodiments 47-48, or the fusion protein of the method of embodiment 49, wherein the protein of interest is a therapeutic protein.

Embodiment 51. The fusion protein of any one of embodiments 22-32, the isolated nucleic acid of any one of embodiments 34-40, the rAAV of any one of embodiments 4146, or the composition of any one of embodiments 47-48, or the fusion protein of the method of embodiment 49, wherein the protein of interest is a transcriptional factor, transcriptional repressor. RNA binding protein. DNA modifying protein, DNA editing protein. Cas protein, DUX4, or an protein carrying an RNA.

Embodiment 52. The fusion protein of any one of embodiments 22-32, the isolated nucleic acid of any one of embodiment, 34-40, the rAAV of any one of embodiments 4146, or the composition of any one of embodiments 47-4841-42, or the fusion protein of the method of embodiment 49, wherein the protein of interest is a nuclear protein.

Embodiment 53. The method of embodiment 49, wherein the subject is mammalian.

Embodiment 54. The method of any one of embodiments 49 or 53, wherein the subject is human.

Embodiment 55. The method of any one of embodiments 49 or 53-54, wherein the subject has a disorder.

Embodiment 56. The method of embodiment 55, wherein the disorder is Facioscapulohumeral dystrophy.

### General Techniques

The practice of the subject matter of the disclosure will employ, unless otherwise indicated, conventional techniques of molecular biology (including recombinant techniques), microbiology, cell biology, biochemistry and immunology, which are within the skill of the art. Such techniques are explained fully in the literature, such as, but without limiting. Molecular Cloning: A Laboratory Manual, second edition (Sambrook, et al., 1989) Cold Spring Harbor Press; Oligonucleotide Synthesis (M. J. Gait, ed., 1984): Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press: Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds. 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999): Immunobiology C. A. Janeway and P. Travers, 1997); Antibodies (P. Finch, 1997); Antibodies: a practical approach (D. Catty, ed., IRL Press, 1988-1989); Monoclonal antibodies: a practical approach (P. Shepherd and C. Dean, eds., Oxford University Press, 2000): Using antibodies: r laboratory manual (E. Harlow and D. Lane (Cold Spring Harbor Laboratory Press, 1999); The Antibodies (M. Zanetti and J. D. Capra, eds., Harwood Academic Publishers, 1995).

### EQUIVALENTS AND SCOPE

It is to be understood that this disclosure is not limited to any or all of the particular embodiments described expressly herein, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents (i.e., any lexicographical definition in the publications and patents cited that is not also expressly repeated in the disclosure should not be treated as such and should not be read as defining any terms appearing in the accompanying claims). If there is a conflict between any of the incorporated references and this disclosure, this disclosure shall control. In addition, any particular embodiment of this disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

In the claims articles such as “a,” “an,” and “the” may mean one or more than one unless indicated to the contrary or otherwise evident from the context. Wherever used herein, a pronoun in a gender (e.g., masculine, feminine, neuter, other, etc. . . . ) the pronoun shall be construed as gender neutral (e.g., construed to refer to all genders equally) regardless of the implied gender unless the context clearly indicates or requires otherwise. Wherever used herein, words used in the singular include the plural, and words used in the plural includes the singular, unless the context clearly indicates or requires otherwise. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permulations in which one or more limitations, elements, clauses, and descriptive term from one or more of the listed claims is introduced into another claim. For example, any claim that is dependent on another claim can be modified to include one or mom limitations found in any other claim that is dependent on the same base claim. Where elements are presented as lists (e.g., in Markush group format), each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described wherein is not intended to be limited to the above Description, but rather is as set forth in the appended claims. Those of ordinary skill in the art will appreciate that various changes and modifications to this description may be made without departing from the spirit or scope of the disclosure, as defined in the following claims.

1. A method comprising administering an isolated nucleic acid to a multinucleate cell, wherein the isolated nucleic acid comprises a sequence encoding a fusion protein, the fusion protein comprising, a protein of interest fused to at least the following migration signals:
(a) at least one nuclear export signal (NES); and
(b) at least one nuclear localization signal (NLS) and/or at least one nucleolar localization signal (NoLS),
optionally, wherein the isolated nucleic acid encodes an amino acid sequence comprising a sequence with at least 70% identity to SEQ ID NO: 13.

2. The method of claim 1, wherein the fusion protein further comprises at least one additional migration signal, wherein the additional migration signal may be identical or distinct from the migration signals used in claim 1, or wherein the fusion protein further comprises at least two additional migration signals, wherein the additional migration signals may be identical or distinct from the migration signals used in claim 1.

3. (canceled)

4. The method of claim 1, wherein the isolated nucleic acid encodes a fusion protein wherein at least one of the migration signals is positioned at the C-terminus of the protein of interest or at the N-terminus of the protein of interest.

5. (canceled)

6. The method of claim 1, wherein the protein of interest is a therapeutic protein, a nuclear protein, or at least one of the following: a transcriptional factor, a transcriptional repressor, an RNA binding protein, a DNA modifying protein, a DNA editing protein, and a Cas protein.

7.-8. (canceled)

9. The method of claim 1, wherein the protein of interest is DUX4, or a variant thereof.

10. The method of claim 1, wherein the protein of interest carries RNA.

11. The method of claim 1, wherein the protein of interest is used to treat Facioscapulohumeral dystrophy.

12. The method of claim 1, wherein the isolated nucleic acid is flanked by adeno-associated virus (AAV) inverted terminal repeats (ITRs).

13. The method of claim 1, wherein the multinucleate cell is a skeletal muscle cell.

14. The method of claim 1, wherein the multinucleate cell is a liver cell.

15. The method of claim 1, wherein the nucleic acid is administered to a subject, optionally wherein the subject is mammalian.

16. The method of claim 1, wherein the subject has a disorder, optionally wherein the disorder is Facioscapulohumeral dystrophy.

17.-21. (canceled)

22. A fusion protein comprising:
(a) a protein of interest; and
(b) at least the following migration signals:
(i) a nuclear export signal (NES);
(ii) a nuclear localization signal (NLS); and
(iii) a nucleolar localization signal (NoLS),
optionally, wherein the fusion protein comprises a sequence with at least 70% identity to SEQ ID NO: 13.

23.-26. (canceled)

27. The fusion protein of claim 22, wherein at least one of the migration signals is linked to the protein of interest via a linker.

28. The fusion protein of claim 22, wherein at least one of the migration signals comprises a sequence with at least 95% identity to any one of SEQ ID NO: 1-6 or 14-570.

29. The fusion protein of claim 22, wherein at least one of the migration signals comprises a sequence with at least 95% identity to SEQ ID NO: 3.

30. The fusion protein of claim 22, wherein at least one of the migration signals comprises a sequence of SEQ ID NO: 1.

31. The fusion protein of claim 22, wherein at least one of the migration signals comprises a sequence of SEQ ID NO: 3.

32.-33. (canceled)

34. An isolated nucleic acid comprising a nucleic acid sequence encoding the fusion protein of claim 22.

35.-40. (canceled)

41. A recombinant adeno-associated virus (rAAV), comprising:
(a) the isolated nucleic acid of claim 34 flanked by adeno-associated virus (AAV) inverted terminal repeats (ITRs); and
(b) an AAV capsid protein.

42.-56. (canceled)


SEQUENCE LISTING (570 sequences)

SEQ ID NO: 1 - protein, 28 aa, Artificial Sequence
  Synthetic polypeptide
APPAQPPSQPQQHYSEGELEEDEDSDDA

SEQ ID NO: 2 - protein, 17 aa, Artificial Sequence
  Synthetic polypeptide
RFEMFRELNEALELKDA

SEQ ID NO: 3 - protein, 8 aa, Artificial Sequence
  Synthetic polypeptide
PKKKRAVE

SEQ ID NO: 4 - protein, 9 aa, Artificial Sequence
  Synthetic polypeptide
MPKKKRAVE

SEQ ID NO: 5 - protein, 7 aa, Artificial Sequence
  Synthetic polypeptide
PKKKRKV

SEQ ID NO: 6 - protein, 7 aa, Artificial Sequence
  Synthetic polypeptide
RKKRKKK

SEQ ID NO: 7 - protein, 244 aa, Artificial Sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGTAAA

SEQ ID NO: 8 - protein, 264 aa, Artificial Sequence [sequence omitted]

SEQ ID NO: 9 - protein, 286 aa, Artificial Sequence
  Synthetic
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 10 - DNA, 6246 bp, Artificial Sequence
  Synthetic
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatgcccaaaaagaaaagagccgtggaaggatccgaattcatggtttctaagggggaagagcttttcacgggagtcgtcccgattctggtggagttggacggggatgttaatggtcacaagttctccgtcagtggtgagggggagggggatgcgacctacggcaaactgactttgaagtttatatgcacaacaggcaaacttcccgtaccctggcctactttggtgactacgcttacatacggggtccagtgcttcagtagatatccagatcacatgaagcagcacgatttttttaaaagcgccatgccagagggctatgttcaagagaggacaattttcttcaaggatgacggcaactacaaaactcgggctgaggtcaaatttgaaggagatacgctggtgaacaggatagaactgaagggaattgacttcaaggaggatggaaatattctcgggcataaattggagtataactacaattctcataacgtttacattatggccgataaacaaaaaaatggtataaaggttaacttcaaaattcggcataacatagaggacgggtcagtgcagctcgcagaccattaccagcaaaatacgccgataggtgatgggccggttcttttgcctgataatcactacctcagcacacagagtgccctcagcaaagacccaaacgaaaaacgagatcatatggtgctcctggaatttgttacagcggcaggaataacactgggaatggacgaactttacaagggtaccgcggccgcagctcctccggcacaacctcccagccaacctcagcagcattatagcgaaggagagttggaggaagatgaagattctgatgacgcgcgcaaaaaacgcaaaaaaaaataagtgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 11 - protein, 292 aa, Artificial Sequence
  Synthetic
MPKKKRAVEGSEFMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGTAAAAPPAQPPSQPQQHYSEGELEEDEDSDDARKKRKKK

SEQ ID NO: 12 - protein, 181 aa, Artificial Sequence
  Synthetic
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQELLASPEFLQQAQPLLETEAPG

SEQ ID NO: 13 - protein, 215 aa, Artificial Sequence
  Synthetic
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQELLASPEFLQQAQPLLETEAPGLEGTRFEAPPAQPPSQPQQHYSEGELEEDEDSDD

SEQ ID NO: 14 - protein, 16 aa, Artificial Sequence
  Synthetic
DDIVFEDFARLRLKGM

SEQ ID NO: 15 - protein, 10 aa, Artificial Sequence
  Synthetic
LIAGIIAMIC

SEQ ID NO: 16 - protein, 63 aa, Artificial Sequence
  Synthetic
SLPPEPMMPMDQTMHPDHTQTVIPYNPSSHESLDQVGEEKEAMNTRESGKASSSLGLQDFDLL

SEQ ID NO: 17 - protein, 11 aa, Artificial Sequence
  Synthetic
MTLGMIWTIIL

SEQ ID NO: 18 - protein, 15 aa, Artificial Sequence
  Synthetic
FEALMRMLDNLGYRT

SEQ ID NO: 19 - protein, 10 aa, Artificial Sequence
  Synthetic
LAQQFEQLSV

SEQ ID NO: 20 - protein, 21 aa, Artificial Sequence
  Synthetic
PFPQDVINKLDKLSVLRLSVS

SEQ ID NO: 21 - protein, 22 aa, Artificial Sequence
  Synthetic
RLGSHTMDFFEMCASLITALAR

SEQ ID NO: 22 - protein, 21 aa, Artificial Sequence
  Synthetic
MSHVAVENVLNLDQQFAGLDL

SEQ ID NO: 23 - protein, 18 aa, Artificial Sequence
  Synthetic
VIQQTLAAIVDAIKLDAI

SEQ ID NO: 24 - protein, 15 aa, Artificial Sequence
  Synthetic
VGGELLDLLGDLNLS

SEQ ID NO: 25 - protein, 19 aa, Artificial Sequence
  Synthetic
VLSAVKVLMKFLELLPKDS

SEQ ID NO: 26 - protein, 10 aa, Artificial Sequence
  Synthetic
LLERLKELNL

SEQ ID NO: 27 - protein, 10 aa, Artificial Sequence
  Synthetic
LTKRIDSLPL

SEQ ID NO: 28 - protein, 21 aa, Artificial Sequence
  Synthetic
ESTPDGFSCSSSLSALSLDEP

SEQ ID NO: 29 - protein, 21 aa, Artificial Sequence
  Synthetic
EGTPINFSTATSLSDLTIESP

SEQ ID NO: 30 - protein, 21 aa, Artificial Sequence
  Synthetic
EDTPVCFSRNSSLSSLSIDSE

SEQ ID NO: 31 - protein, 15 aa, Artificial Sequence
  Synthetic
LWVRLYVLELYCIIL

SEQ ID NO: 32 - protein, 15 aa, Artificial Sequence
  Synthetic
DVKSLESALKDLKIK

SEQ ID NO: 33 - protein, 16 aa, Artificial Sequence
  Synthetic
LLRNEVAQLKQLLLAH

SEQ ID NO: 34 - protein, 12 aa, Artificial Sequence
  Synthetic
LEELCAARRLSL

SEQ ID NO: 35 - protein, 11 aa, Artificial Sequence
  Synthetic
ELITFINALKL

SEQ ID NO: 36 - protein, 19 aa, Artificial Sequence
  Synthetic
QLVEELLKIICAFQLDTGL

SEQ ID NO: 37 - protein, 11 aa, Artificial Sequence
  Synthetic
DLSDLTFLEVA

SEQ ID NO: 38 - protein, 15 aa, Artificial Sequence
  Synthetic
RDLERAMTTLKLWES

SEQ ID NO: 39 - protein, 18 aa, Artificial Sequence
  Synthetic
AGDILALVFGLLFAVTSV

SEQ ID NO: 40 - protein, 18 aa, Artificial Sequence
  Synthetic
LDSSLANLVGNLGIGNGT

SEQ ID NO: 41 - protein, 19 aa, Artificial Sequence
  Synthetic
SSTSGLEQDVAQLNIAEQN

SEQ ID NO: 42 - protein, 62 aa, Artificial Sequence
  Synthetic
ASSLRDYAASTMTEFLGMFGYDDQNTRDELARKISFEKLHAGSTPEAATSSMLPTSEDTLSK

SEQ ID NO: 43 - protein, 10 aa, Artificial Sequence
  Synthetic
LEMFGPEGAL

SEQ ID NO: 44 - protein, 6 aa, Artificial Sequence
  Synthetic
HLVLIG

SEQ ID NO: 45 - protein, 10 aa, Artificial Sequence
  Synthetic
LRKLCERLRG

SEQ ID NO: 46 - protein, 10 aa, Artificial Sequence
  Synthetic
LLDKLLDLNP

SEQ ID NO: 47 - protein, 6 aa, Artificial Sequence
  Synthetic
EQCERA

SEQ ID NO: 48 - protein, 10 aa, Artificial Sequence
  Synthetic
VLRMMVGVNI

SEQ ID NO: 49 - protein, 10 aa, Artificial Sequence
  Synthetic
FVKVLEKVDV

SEQ ID NO: 50 - protein, 23 aa, Artificial Sequence
  Synthetic
PNCPFLENSLETLRFSISNLSMQ

SEQ ID NO: 51 - protein, 8 aa, Artificial Sequence
  Synthetic
LGALWLAL

SEQ ID NO: 52 - protein, 14 aa, Artificial Sequence
  Synthetic
IHTPVAIIELELGK

SEQ ID NO: 53 - protein, 17 aa, Artificial Sequence
  Synthetic
EVKVNQILKTLSEVEES

SEQ ID NO: 54 - protein, 45 aa, Artificial Sequence
  Synthetic
DLLGTDQDNLDLANVNLMLELLVQKKKQLEAESHAAQLQILMEFL

SEQ ID NO: 55 - protein, 10 aa, Artificial Sequence
  Synthetic
LAALNHISAL

SEQ ID NO: 56 - protein, 50 aa, Artificial Sequence
  Synthetic
LKVIEENVCPKPAQVEPSSPSPMETSGCLPDELCQAFSDVLIHVKDVDAD

SEQ ID NO: 57 - protein, 12 aa, Artificial Sequence
  Synthetic
DLCQAFSDVILA

SEQ ID NO: 58 - protein, 22 aa, Artificial Sequence
  Synthetic
MSHVAVENALGLDQQFAGLDLN

SEQ ID NO: 59 - protein, 15 aa, Artificial Sequence
  Synthetic
PFGQALRPLLDSIQI

SEQ ID NO: 60 - protein, 9 aa, Artificial Sequence
  Synthetic
LSTLDQLRL

SEQ ID NO: 61 - protein, 13 aa, Artificial Sequence
  Synthetic
SLEGAVSEISLRD

SEQ ID NO: 62 - protein, 11 aa, Artificial Sequence
  Synthetic
LYPELRRILTI

SEQ ID NO: 63 - protein, 27 aa, Artificial Sequence
  Synthetic
YLAESSGPARGRGRHPGKGVKSPGEKS

SEQ ID NO: 64 - protein, 26 aa, Artificial Sequence
  Synthetic
TPSAPRPALGRPPVKRRLDLETDHQY

SEQ ID NO: 65 - protein, 10 aa, Artificial Sequence
  Synthetic
MVLTREELVI

SEQ ID NO: 66 - protein, 20 aa, Artificial Sequence
  Synthetic
VSQIFPDSVMLAVQEGIDLL

SEQ ID NO: 67 - protein, 19 aa, Artificial Sequence
  Synthetic
FDIDEAEEGVKDLKIESDV

SEQ ID NO: 68 - protein, 24 aa, Artificial Sequence
  Synthetic
RIDRDVQILNHILDDIEFFITKLQ

SEQ ID NO: 69 - protein, 11 aa, Artificial Sequence
  Synthetic
LCNCALEELRL

SEQ ID NO: 70 - protein, 10 aa, Artificial Sequence
  Synthetic
LWEFIRDILI

SEQ ID NO: 71 - protein, 10 aa, Artificial Sequence
  Synthetic
IHKKFSSIQM

SEQ ID NO: 72 - protein, 19 aa, Artificial Sequence
  Synthetic
RLSHLRSEEVHWLHVDMGV

SEQ ID NO: 73 - protein, 21 aa, Artificial Sequence
  Synthetic
FLQVRKYSLDLASLILYAYQL

SEQ ID NO: 74 - protein, 27 aa, Artificial Sequence
  Synthetic
DLNALLLEVEGPLCKKLSLSKVIDCDS

SEQ ID NO: 75 - protein, 22 aa, Artificial Sequence
  Synthetic
PQVTVDVLQRMLIFALDALAAG

SEQ ID NO: 76 - protein, 17 aa, Artificial Sequence
  Synthetic
LADLKVSIENMGLYEDL

SEQ ID NO: 77 - protein, 21 aa, Artificial Sequence
  Synthetic
LSPGLIKKFQFLMFRLFSEAR

SEQ ID NO: 78 - protein, 31 aa, Artificial Sequence
  Synthetic
GNEDIISRLQEMVADLELQQDLIVPLGHTPS

SEQ ID NO: 79 - protein, 10 aa, Artificial Sequence
  Synthetic
LVVLPLELKL

SEQ ID NO: 80 - protein, 10 aa, Artificial Sequence
  Synthetic
FVEKLQDIQQ

SEQ ID NO: 81 - protein, 17 aa, Artificial Sequence
  Synthetic
EVDQLRLERLQIDEQLR

SEQ ID NO: 82 - protein, 10 aa, Artificial Sequence
  Synthetic
LHGTMRPLSL

SEQ ID NO: 83 - protein, 17 aa, Artificial Sequence
  Synthetic
QILPKVLHKRTLGLSAM

SEQ ID NO: 84 - protein, 33 aa, Artificial Sequence
  Synthetic
MVNFVSVGLFRCLPVPCPEDLLVEELVDGLLSL

SEQ ID NO: 85 - protein, 26 aa, Artificial Sequence
  Synthetic
EAQTCENEEAETVTAMASLSVGVKPA

SEQ ID NO: 86 - protein, 42 aa, Artificial Sequence
  Synthetic
EEAETVSAMALLSVGAEQAQAAAAREHSPRPAEEPMEQEPAL

SEQ ID NO: 87 - protein, 15 aa, Artificial Sequence
  Synthetic
LSFDESLALCVIREI

SEQ ID NO: 88 - protein, 12 aa, Artificial Sequence
  Synthetic
LQLPPLERLTLD

SEQ ID NO: 89 - protein, 10 aa, Artificial Sequence
  Synthetic
LVDKFMKLDL

SEQ ID NO: 90 - protein, 19 aa, Artificial Sequence
  Synthetic
DTDIQELSEQIHRLLLQPV

SEQ ID NO: 91 - protein, 10 aa, Artificial Sequence
  Synthetic
ISPRLDAIKL

SEQ ID NO: 92 - protein, 23 aa, Artificial Sequence
  Synthetic
ARSFEMTEFNQALEEIKGQVVEN

SEQ ID NO: 93 - protein, 9 aa, Artificial Sequence
  Synthetic
DIELTLRLN

SEQ ID NO: 94 - protein, 13 aa, Artificial Sequence
  Synthetic
EELQDLVDQLGFL

SEQ ID NO: 95 - protein, 14 aa, Artificial Sequence
  Synthetic
IEAALSDALAALQI

SEQ ID NO: 96 - protein, 17 aa, Artificial Sequence
  Synthetic
MATLEKLMKAFESLKSF

SEQ ID NO: 97 - protein, 51 aa, Artificial Sequence
  Synthetic
MVAEMVESLQSVLALGHKRNSGVPAFLTPLLRNIIISLARLPLVNSYTRVP

SEQ ID NO: 98 - protein, 55 aa, Artificial Sequence
  Synthetic
MFQAAERPQEWAMEGPRDGLKKERLLDDRHDSGLDSMKDEEYEQMVKELQEIRLE

SEQ ID NO: 99 - protein, 14 aa, Artificial Sequence
  Synthetic
VIDYIRDLQLELNS

SEQ ID NO: 100 - protein, 17 aa, Artificial Sequence
  Synthetic
LTTLNTDISILSLQASE

SEQ ID NO: 101 - protein, 26 aa, Artificial Sequence
  Synthetic
VSKYPLMEEIGFLVLGMRVYHVHSDS

SEQ ID NO: 102 - protein, 15 aa, Artificial Sequence
  Synthetic
QEDILDELLGNMVLA

SEQ ID NO: 103 - protein, 11 aa, Artificial Sequence
  Synthetic
LQRMLPSLSLT

SEQ ID NO: 104 - protein, 10 aa, Artificial Sequence
  Synthetic
LDRKLLELLW

SEQ ID NO: 105 - protein, 44 aa, Artificial Sequence
  Synthetic
RCHSLTPNFLQMQLQKCEILQSDSRCKDYLVKIFEELTLHKPTQ

SEQ ID NO: 106 - protein, 31 aa, Artificial Sequence
  Synthetic
IPYSINMNVFLPDITHLRTGLYKSQRPCVTQ

SEQ ID NO: 107 - protein, 16 aa, Artificial Sequence
  Synthetic
MDVLPMCSIFQELQIV

SEQ ID NO: 108 - protein, 10 aa, Artificial Sequence
  Synthetic
MVPLVIKLRL

SEQ ID NO: 109 - protein, 25 aa, Artificial Sequence
  Synthetic
FKPDMNPALREVLEALEDEAYVVND

SEQ ID NO: 110 - protein, 12 aa, Artificial Sequence
  Synthetic
ALREVLEALEDE

SEQ ID NO: 111 - protein, 13 aa, Artificial Sequence
  Synthetic
LEKVTNTLSSLKF

SEQ ID NO: 112 - protein, 12 aa, Artificial Sequence
  Synthetic
LEQELQQLSLEL

SEQ ID NO: 113 - protein, 12 aa, Artificial Sequence
  Synthetic
LFGDTIAYLLSL

SEQ ID NO: 114 - protein, 10 aa, Artificial Sequence
  Synthetic
LLYCLMVMYL

SEQ ID NO: 115 - protein, 14 aa, Artificial Sequence
  Synthetic
QLLQEKLEKLTKLK

SEQ ID NO: 116 - protein, 24 aa, Artificial Sequence
  Synthetic
DKERWEDVKEEMTSALATMRVDYE

SEQ ID NO: 117 - protein, 13 aa, Artificial Sequence
  Synthetic
ALQKKLEELELDE

SEQ ID NO: 118 - protein, 14 aa, Artificial Sequence
  Synthetic
VPEVEALLARLRAL

SEQ ID NO: 119 - protein, 9 aa, Artificial Sequence
  Synthetic
DLVLLSLVL

SEQ ID NO: 120 - protein, 10 aa, Artificial Sequence
  Synthetic
QLQQKLLWLL

SEQ ID NO: 121 - protein, 10 aa, Artificial Sequence
  Synthetic
LVIAMDQLNL

SEQ ID NO: 122 - protein, 16 aa, Artificial Sequence
  Synthetic
PLPVLGLGGLRISSDS

SEQ ID NO: 123 - protein, 10 aa, Artificial Sequence
  Synthetic
LFDLAMLALD

SEQ ID NO: 124 - protein, 8 aa, Artificial Sequence
  Synthetic
LHLVGVNV

SEQ ID NO: 125 - protein, 20 aa, Artificial Sequence
  Synthetic
ASLTKLFECMTLAYSGKLVS

SEQ ID NO: 126 - protein, 14 aa, Artificial Sequence
  Synthetic
DIQELSEQIHRLLL

SEQ ID NO: 127 - protein, 10 aa, Artificial Sequence
  Synthetic
LEIALRNLNL

SEQ ID NO: 128 - protein, 59 aa, Artificial Sequence
  Synthetic
DEMDSGTMVRAVGDEMGTVRVASTMTDGANTMIEHDDTLPSQLGTMVINAEDEEEEGTM

SEQ ID NO: 129 - protein, 31 aa, Artificial Sequence
  Synthetic
DGDYEFLKSWTVEDLQKRLLALDPMMEQEIE

SEQ ID NO: 130 - protein, 10 aa, Artificial Sequence
  Synthetic
PVSKITFVTL

SEQ ID NO: 131 - protein, 10 aa, Artificial Sequence
  Synthetic
LPSPLASLTL

SEQ ID NO: 132 - protein, 9 aa, Artificial Sequence
  Synthetic
LCLSDLSLL

SEQ ID NO: 133 - protein, 13 aa, Artificial Sequence
  Synthetic
IQDGLLKMLSLVL

SEQ ID NO: 134 - protein, 10 aa, Artificial Sequence
  Synthetic
ITFIFKSLGL

SEQ ID NO: 135 - protein, 9 aa, Artificial Sequence
  Synthetic
LEWLRRLSL

SEQ ID NO: 136 - protein, 20 aa, Artificial Sequence
  Synthetic
VQVVADVLSKLLVVGITDPD

SEQ ID NO: 137 - protein, 40 aa, Artificial Sequence
  Synthetic
LAPNVAALLFGGNVAVRELADSYEITYNYKMTVPKSDPNV

SEQ ID NO: 138 - protein, 34 aa, Artificial Sequence
  Synthetic
CTLSDSGRISYTVEFSLPTHHTVRLIRVTASPSA

SEQ ID NO: 139 - protein, 28 aa, Artificial Sequence
  Synthetic
GLPKNVKEKLLSLKTLQSELFEVEKEFQ

SEQ ID NO: 140 - protein, 26 aa, Artificial Sequence
  Synthetic
KKTISPEHVIQALESLGFGSYISEVK

SEQ ID NO: 141 - protein, 10 aa, Artificial Sequence
  Synthetic
ILMRMSKMQL

SEQ ID NO: 142 - protein, 10 aa, Artificial Sequence
  Synthetic
MVTRFESLKI

SEQ ID NO: 143 - protein, 12 aa, Artificial Sequence
  Synthetic
YIWALTQTLRIA

SEQ ID NO: 144 - protein, 12 aa, Artificial Sequence
  Synthetic
SAIVAAINALTT

SEQ ID NO: 145 - protein, 85 aa, Artificial Sequence
  Synthetic
DAERNRPLNGGSEPESNSALQEDEREKKDELQTESWSTKHEIANSDGLQDSSEELPRKLLLTEFRSLVVSNHNSTSRNLCVNECG

SEQ ID NO: 146 - protein, 50 aa, Artificial Sequence
  Synthetic
KEHKDIDASLDYNSRAQKQEMERAEKDYELFLQELEEDAELRQSVNLYKN

SEQ ID NO: 147 - protein, 14 aa, Artificial Sequence
  Synthetic
LDLPDALLPDLPKL

SEQ ID NO: 148 - protein, 25 aa, Artificial Sequence
  Synthetic
LLPLAEADKVRLSYLHIMSLACIYT

SEQ ID NO: 149 - protein, 11 aa, Artificial Sequence
  Synthetic
DLCLAVEEVSL

SEQ ID NO: 150 - protein, 10 aa, Artificial Sequence
  Synthetic
LKRRLSTLYL

SEQ ID NO: 151 - protein, 10 aa, Artificial Sequence
  Synthetic
LRNQLTALRI

SEQ ID NO: 152 - protein, 11 aa, Artificial Sequence
  Synthetic
LLLPLMRNLEM

SEQ ID NO: 153 - protein, 11 aa, Artificial Sequence
  Synthetic
LVSLIRLKSKL

SEQ ID NO: 154 - protein, 27 aa, Artificial Sequence
  Synthetic
DEMTKKFGTLTIHDTEKYASQPELCNN

SEQ ID NO: 155 - protein, 10 aa, Artificial Sequence
  Synthetic
LCPDLPELDL

SEQ ID NO: 156 - protein, 10 aa, Artificial Sequence
  Synthetic
LVDSLQQLRL

SEQ ID NO: 157 - protein, 9 aa, Artificial Sequence
  Synthetic
LELFVLRLA

SEQ ID NO: 158 - protein, 9 aa, Artificial Sequence
  Synthetic
LQRIFYLKL

SEQ ID NO: 159 - protein, 9 aa, Artificial Sequence
  Synthetic
LKSLGELGL

SEQ ID NO: 160 - protein, 9 aa, Artificial Sequence
  Synthetic
LSKIATLLL

SEQ ID NO: 161 - protein, 9 aa, Artificial Sequence
  Synthetic
LLLAGLPLL

SEQ ID NO: 162 - protein, 25 aa, Artificial Sequence
  Synthetic
HPSTPKRHTVLYISPPPEDLLDNSR

SEQ ID NO: 163 - protein, 11 aa, Artificial Sequence
  Synthetic
VLSQRIGLMDV

SEQ ID NO: 164 - protein, 15 aa, Artificial Sequence
  Synthetic
LRHELVEDAVYENPL

SEQ ID NO: 165 - protein, 16 aa, Artificial Sequence
  Synthetic
SLEEELDVLVLDDEGG

SEQ ID NO: 166 - protein, 18 aa, Artificial Sequence
  Synthetic
LTELEISSIFSHCCSLLI

SEQ ID NO: 167 - protein, 14 aa, Artificial Sequence
  Synthetic
EMFRELNEALELKD

SEQ ID NO: 168 - protein, 19 aa, Artificial Sequence
  Synthetic
NFEILMKLKESLELMELVP

SEQ ID NO: 169 - protein, 13 aa, Artificial Sequence
  Synthetic
LTLLLDEFENMSV

SEQ ID NO: 170 - protein, 15 aa, Artificial Sequence
  Synthetic
VVMEFLEGGALTDIV

SEQ ID NO: 171 - protein, 10 aa, Artificial Sequence
  Synthetic
LKGFLDRLLV

SEQ ID NO: 172 - protein, 18 aa, Artificial Sequence
  Synthetic
VYNLVCVALGNLEIREIR

SEQ ID NO: 173 - protein, 19 aa, Artificial Sequence
  Synthetic
ESFDIDDLCSKLKNKAKCS

SEQ ID NO: 174 - protein, 18 aa, Artificial Sequence
  Synthetic
MKPALFNVLCEIKEKTVL

SEQ ID NO: 175 - protein, 13 aa, Artificial Sequence
  Synthetic
ILKKVLEALKDLI

SEQ ID NO: 176 - protein, 11 aa, Artificial Sequence
  Synthetic
LLKDLPELALD

SEQ ID NO: 177 - protein, 11 aa, Artificial Sequence
  Synthetic
VAEMLRDLNLG

SEQ ID NO: 178 - protein, 10 aa, Artificial Sequence
  Synthetic
LELEALRLSL

SEQ ID NO: 179 - protein, 11 aa, Artificial Sequence
  Synthetic
LQDALRRLLGL

SEQ ID NO: 180 - protein, 11 aa, Artificial Sequence
  Synthetic
FGETLRAAVTL

SEQ ID NO: 181 - protein, 9 aa, Artificial Sequence
  Synthetic
LDEFNELAI

SEQ ID NO: 182 - protein, 11 aa, Artificial Sequence
  Synthetic
VIEKLQHELSL

SEQ ID NO: 183 - protein, 50 aa, Artificial Sequence
  Synthetic
RVREKQYQDTIGKLQKENNELLEQLEMLQAQLKNSTLDSPKEVEVNSEVV

SEQ ID NO: 184 - protein, 18 aa, Artificial Sequence
  Synthetic
SLWGEHILALKNLKLDKM

SEQ ID NO: 185 - protein, 15 aa, Artificial Sequence
  Synthetic
NELALKLAGLDINKT

SEQ ID NO: 186 - protein, 14 aa, Artificial Sequence
  Synthetic
ELKDFLKELNIQVD

SEQ ID NO: 187 - protein, 10 aa, Artificial Sequence
  Synthetic
ISGFLAALPL

SEQ ID NO: 188 - protein, 19 aa, Artificial Sequence
  Synthetic
REELWKKLEELKLKKALEK

SEQ ID NO: 189 - protein, 16 aa, Artificial Sequence
  Synthetic
YEEQLVALFGSSMDLR

SEQ ID NO: 190 - protein, 10 aa, Artificial Sequence
  Synthetic
IIELLKGLDL

SEQ ID NO: 191 - protein, 10 aa, Artificial Sequence
  Synthetic
LSLNLFALRI

SEQ ID NO: 192 - protein, 8 aa, Artificial Sequence
  Synthetic
LTLSSLTL

SEQ ID NO: 193 - protein, 9 aa, Artificial Sequence
  Synthetic
VEVLREIQL

SEQ ID NO: 194 - protein, 10 aa, Artificial Sequence
  Synthetic
IMSQFRKLLM

SEQ ID NO: 195 - protein, 16 aa, Artificial Sequence
  Synthetic
EVDNLPEDMKRLHLDD

SEQ ID NO: 196 - protein, 18 aa, Artificial Sequence
  Synthetic
KVAEKLEALSVKEETKED

SEQ ID NO: 197 - protein, 16 aa, Artificial Sequence
  Synthetic
EEILKLLMELVFRLVC

SEQ ID NO: 198 - protein, 10 aa, Artificial Sequence
  Synthetic
LSEALLQLQF

SEQ ID NO: 199 - protein, 10 aa, Artificial Sequence
  Synthetic
LEDLVRHMSL

SEQ ID NO: 200 - protein, 14 aa, Artificial Sequence
  Synthetic
ALSAQLYSSLSLDS

SEQ ID NO: 201 - protein, 42 aa, Artificial Sequence
  Synthetic
EKGNLPELEKLEINGNRLDEDSDALDLLQSKFDDLEVDDFEE

SEQ ID NO: 202 - protein, 37 aa, Artificial Sequence
  Synthetic
GDVFGPELDTLLDSLSLVQGGLSGSGVPSELPQLIPV

SEQ ID NO: 203 - protein, 42 aa, Artificial Sequence
  Synthetic
DGFNELIPEDLVTVFDERELELLIGGIAEIDIEDWKKHTDYR

SEQ ID NO: 204 - protein, 23 aa, Artificial Sequence
  Synthetic
QMLSKEVDACVTDLLKELVRFQD

SEQ ID NO: 205 - protein, 15 aa, Artificial Sequence
  Synthetic
QDQFHKMVELTVAAR

SEQ ID NO: 206 - protein, 28 aa, Artificial Sequence
  Synthetic
QYESNENVVLVCSTIVCSFGKQVVEKVE

SEQ ID NO: 207 - protein, 16 aa, Artificial Sequence
  Synthetic
LEPDLSEEVSARLRLG

SEQ ID NO: 208 - protein, 16 aa, Artificial Sequence
  Synthetic
RRMQEMITRMQAQMQI

SEQ ID NO: 209 - protein, 19 aa, Artificial Sequence
  Synthetic
TGEQELESLVLKLSVLKDF

SEQ ID NO: 210 - protein, 19 aa, Artificial Sequence
  Synthetic
LESLVLKLSVLKDFLSGIQ

SEQ ID NO: 211 - protein, 54 aa, Artificial Sequence
  Synthetic
SRDAARCRRSKETEIFMELSAALPLKTDDVNQLDKASVMRITIAFLKIREMLQF

SEQ ID NO: 212 - protein, 19 aa, Artificial Sequence
  Synthetic
DMDFLRNLFSQTLSLGSQK

SEQ ID NO: 213 - protein, 9 aa, Artificial Sequence
  Synthetic
LTKMCTIRM

SEQ ID NO: 214 - protein, 10 aa, Artificial Sequence
  Synthetic
GIDLSGLTLQ

SEQ ID NO: 215 - protein, 12 aa, Artificial Sequence
  Synthetic
ELIIGGLDKIDL

SEQ ID NO: 216 - protein, 70 aa, Artificial Sequence
  Synthetic
SPKAVELTSLSDEDSGKSSQPPSPPSPAPSSFSSTSASSLEAEAFIAFPGLGQLPKQLARLSVAKDPQSR

SEQ ID NO: 217 - protein, 15 aa, Artificial Sequence
  Synthetic
MEELSQALASSFSVS

SEQ ID NO: 218 - protein, 32 aa, Artificial Sequence
  Synthetic
ESNGPVKVWGSIKGLTEGLHGFHVHEFGDNTA

SEQ ID NO: 219 - protein, 12 aa, Artificial Sequence
  Synthetic
LSKTLGKLWRLL

SEQ ID NO: 220 - protein, 15 aa, Artificial Sequence
  Synthetic
EISKQLGKRWKNLTE

SEQ ID NO: 221 - protein, 15 aa, Artificial Sequence
  Synthetic
EISKQLGRRWKMLTE

SEQ ID NO: 222 - protein, 14 aa, Artificial Sequence
  Synthetic
ELSKTLGKLWRLLN

SEQ ID NO: 223 - protein, 12 aa, Artificial Sequence
  Synthetic
LEKQINDLQIDK

SEQ ID NO: 224 - protein, 19 aa, Artificial Sequence
  Synthetic
SASDDLEALGTLSLGTTEE

SEQ ID NO: 225 - protein, 10 aa, Artificial Sequence
  Synthetic
IYPWMKRVHL

SEQ ID NO: 226 - protein, 9 aa, Artificial Sequence
  Synthetic
LLLKKMYLM

SEQ ID NO: 227 - protein, 13 aa, Artificial Sequence
  Synthetic
WDRTFSLFQQLIQ

SEQ ID NO: 228 - protein, 22 aa, Artificial Sequence
  Synthetic
MEESTNGSLAAEFRHLQLKEQK

SEQ ID NO: 229 - protein, 13 aa, Artificial Sequence
  Synthetic
EERIVELFRNLMK

SEQ ID NO: 230 - protein, 11 aa, Artificial Sequence
  Synthetic
LSAEFKHLTLR

SEQ ID NO: 231 - protein, 14 aa, Artificial Sequence
  Synthetic
QLTTLAEKLLGPGV

SEQ ID NO: 232 - protein, 11 aa, Artificial Sequence
  Synthetic
INQMFSVQLSL

SEQ ID NO: 233 - protein, 17 aa, Artificial Sequence
  Synthetic
EATEAQLNNSMAALNVN

SEQ ID NO: 234 - protein, 16 aa, Artificial Sequence
  Synthetic
VKKQFEELTLGEFLKL

SEQ ID NO: 235 - protein, 15 aa, Artificial Sequence
  Synthetic
YKRIEELLYKISLTT

SEQ ID NO: 236 - protein, 39 aa, Artificial Sequence
  Synthetic
NPASAPPPLPPPGQQVIHVTQDLDTDLEALFNSVMNPKP

SEQ ID NO: 237 - protein, 24 aa, Artificial Sequence
  Synthetic
HVLSSSAGNSAPNSPMAMLHIGSN

SEQ ID NO: 238 - protein, 40 aa, Artificial Sequence
  Synthetic
MANSANTNTVPKLYRSVIEDVINDVRDIFLDDGVDEQVLM

SEQ ID NO: 239 - protein, 19 aa, Artificial Sequence
  Synthetic
QPDASKADPLPVLENLTLK

SEQ ID NO: 240 - protein, 11 aa, Artificial Sequence
  Synthetic
ILRDFFELRLK

SEQ ID NO: 241 - protein, 13 aa, Artificial Sequence
  Synthetic
FILEKIDGKIIIE

SEQ ID NO: 242 - protein, 11 aa, Artificial Sequence
  Synthetic
ETVQDILKEFF

SEQ ID NO: 243 - protein, 34 aa, Artificial Sequence
  Synthetic
RISYQPENIQPNRHVANIVEKLREVKLSPEEGQK

SEQ ID NO: 244 - protein, 40 aa, Artificial Sequence
  Synthetic
QRLQGLPPDRGIIRPGSLDAEIDSLTSMLADLDGGRSHAP

SEQ ID NO: 245 - protein, 12 aa, Artificial Sequence
  Synthetic
WMAIAGKIRSDL

SEQ ID NO: 246 - protein, 10 aa, Artificial Sequence
  Synthetic
CILCQLLLLY

SEQ ID NO: 247 - protein, 15 aa, Artificial Sequence
  Synthetic
TTEELELELETLDIN

SEQ ID NO: 248 - protein, 39 aa, Artificial Sequence
  Synthetic
TDPFLAMQVQELTRSMANLTFKQRRDAPPEGPSAKKPKK

SEQ ID NO: 249 - protein, 14 aa, Artificial Sequence
  Synthetic
ELDTNFFTLYVAQG

SEQ ID NO: 250 - protein, 10 aa, Artificial Sequence
  Synthetic
LERLFGRLRI

SEQ ID NO: 251 - protein, 19 aa, Artificial Sequence
  Synthetic
MDGAIASGVSKFATLSLHD

SEQ ID NO: 252 - protein, 12 aa, Artificial Sequence
  Synthetic
IEVEASDLSLSL

SEQ ID NO: 253 - protein, 19 aa, Artificial Sequence
  Synthetic
SSLQELVQQFEALPGDLVG

SEQ ID NO: 254 - protein, 15 aa, Artificial Sequence
  Synthetic
EQLERLRKDMGSVAL

SEQ ID NO: 255 - protein, 9 aa, Artificial Sequence
  Synthetic
LRLGSQIFI

SEQ ID NO: 256 - protein, 10 aa, Artificial Sequence
  Synthetic
LQLVVLRDSK

SEQ ID NO: 257 - protein, 10 aa, Artificial Sequence
  Synthetic
LFGPIADIAL

SEQ ID NO: 258 - protein, 11 aa, Artificial Sequence
  Synthetic
LEKLANELPDL

SEQ ID NO: 259 - protein, 17 aa, Artificial Sequence
  Synthetic
LTMKEVEELELLTQKLM

SEQ ID NO: 260 - protein, 28 aa, Artificial Sequence
  Synthetic
APPAQPPSQPQQHYSEGELEEDEDSDDA

SEQ ID NO: 261 - protein, 17 aa, Artificial Sequence
  Synthetic
RFEMFRELNEALELKDA

SEQ ID NO: 262 - protein, 24 aa, Artificial Sequence
  Synthetic
KRKRKRKRKRKRKRKRKRKRKKKL

SEQ ID NO: 263 - protein, 7 aa, Artificial Sequence
  Synthetic
PKKKRKV

SEQ ID NO: 264 - protein, 9 aa, Artificial Sequence
  Synthetic
PKKNRLRRP

SEQ ID NO: 265 - protein, 20 aa, Artificial Sequence
  Synthetic
PKRPRDRHDGELGGRKRARG

SEQ ID NO: 266 - protein, 8 aa, Artificial Sequence
  Synthetic
PLLKKIKQ

SEQ ID NO: 267 - protein, 7 aa, Artificial Sequence
  Synthetic
PNKKKRK

SEQ ID NO: 268 - protein, 8 aa, Artificial Sequence
  Synthetic
PPQKKIKS

SEQ ID NO: 269 - protein, 13 aa, Artificial Sequence
  Synthetic
PPRIYPQLPSAPT

SEQ ID NO: 270 - protein, 9 aa, Artificial Sequence
  Synthetic
PPRKKRTVV

SEQ ID NO: 271 - protein, 9 aa, Artificial Sequence
  Synthetic
PPVKRERTS

SEQ ID NO: 272 - protein, 7 aa, Artificial Sequence
  Synthetic
PQPKKKP

SEQ ID NO: 273 - protein, 8 aa, Artificial Sequence
  Synthetic
PQSRKKLR

SEQ ID NO: 274 - protein, 14 aa, Artificial Sequence
  Synthetic
PRGRRQPIPKARQP

SEQ ID NO: 275 - protein, 8 aa, Artificial Sequence
  Synthetic
PRPRKIPR

SEQ ID NO: 276 - protein, 6 aa, Artificial Sequence
  Synthetic
PRRGPR

SEQ ID NO: 277 - protein, 5 aa, Artificial Sequence
  Synthetic
PRRRK

SEQ ID NO: 278 - protein, 10 aa, Artificial Sequence
  Synthetic
PYLNKRKGKP

SEQ ID NO: 279 - protein, 7 aa, Artificial Sequence
  Synthetic
PKKARED

SEQ ID NO: 280 - protein, 10 aa, Artificial Sequence
  Synthetic
PAKRARRGYK

SEQ ID NO: 281 - protein, 16 aa, Artificial Sequence
  Synthetic
  misc_feature (3)..(4): Xaa can be any naturally occurring amino acid
  misc_feature (5)..(12): Xaa can be any naturally occurring amino acid
  misc_feature (5)..(12): may not be present
KRXXXXXXXXXXKKKL

SEQ ID NO: 282 - protein, 7 aa, Artificial Sequence
  Synthetic
KSKKKAQ

SEQ ID NO: 283 - protein, 7 aa, Artificial Sequence
  Synthetic
KTRKHRG

SEQ ID NO: 284 - protein, 21 aa, Artificial Sequence
  Synthetic
KVNSRKRRKEVPGPNGATEED

SEQ ID NO: 285 - protein, 18 aa, Artificial Sequence
  Synthetic
KVTKRKHDNEGSGSKRPK

SEQ ID NO: 286 - protein, 35 aa, Artificial Sequence
  Synthetic
  misc_feature (9)..(11): may not be present
  misc_feature (12)..(23): Xaa can be any naturally occurring amino acid
  misc_feature (24)..(28): may not be present
  misc_feature (24)..(28): Xaa can be any naturally occurring amino acid
  misc_feature (32)..(33): Xaa can be any naturally occurring amino acid
  misc_feature (34)..(34): may not be present
  misc_feature (34)..(34): Xaa can be any naturally occurring amino acid
KRKRKRKRKRKXXXXXXXXXXXXXXXXXRKKXXXK

SEQ ID NO: 287 - protein, 13 aa, Artificial Sequence
  Synthetic
LEKKVKKKFDWCA

SEQ ID NO: 288 - protein, 13 aa, Artificial Sequence
  Synthetic
LKDVRKRKLGPGH

SEQ ID NO: 289 - protein, 7 aa, Artificial Sequence
  Synthetic
LKRKLQR

SEQ ID NO: 290 - protein, 9 aa, Artificial Sequence
  Synthetic
LKRPRSPSS

SEQ ID NO: 291 - protein, 42 aa, Artificial Sequence
  Synthetic
MAPSAKATAAKKAVVKGTNGKKALKVRTSATFRLPKTLKLAR

SEQ ID NO: 292 - protein, 13 aa, Artificial Sequence
  Synthetic
MNKIPIKDLLNPG

SEQ ID NO: 293 - protein, 18 aa, Artificial Sequence
  Synthetic
MPKTRRRPRRSQRKRPPT

SEQ ID NO: 294 - protein, 30 aa, Artificial Sequence
  Synthetic
MPTEERVRKRKESNRESARRSRYRKAAHLK

SEQ ID NO: 295 - protein, 38 aa, Artificial Sequence
  Synthetic
NQSSNFGPMKGGNFGGRSSGPYGGGGQYFAKPRNQGGY

SEQ ID NO: 296 - protein, 9 aa, Artificial Sequence
  Synthetic
PAAKRVKLD

SEQ ID NO: 297 - protein, 6 aa, Artificial Sequence
  Synthetic
QRKRQK

SEQ ID NO: 298 - protein, 23 aa, Artificial Sequence
  Synthetic
RAIKRRPGLDFDDDGEGNSKFLR

SEQ ID NO: 299 - protein, 13 aa, Artificial Sequence
  Synthetic
REKKEKEQKEKCA

SEQ ID NO: 300 - protein, 31 aa, Artificial Sequence
  Synthetic
  misc_feature (5)..(26): Xaa can be any naturally occurring amino acid
  misc_feature (6)..(26): may not be present
RRPSXXXXXXXXXXXXXXXXXXXXXXRRKRQ

SEQ ID NO: 301 - protein, 18 aa, Artificial Sequence
  Synthetic
  misc_feature (4)..(14): Xaa can be any naturally occurring amino acid
  misc_feature (5)..(14): may not be present
RRRXXXXXXXXXXXKRRK

SEQ ID NO: 302 - protein, 7 aa, Artificial Sequence
  Synthetic
RRSMKRK

SEQ ID NO: 303 - protein, 7 aa, Artificial Sequence
  Synthetic
RVHPYQR

SEQ ID NO: 304 - protein, 26 aa, Artificial Sequence
  Synthetic
SANKVTKNKSNSSPYLNKRGKPGPDS

SEQ ID NO: 305 - protein, 13 aa, Artificial Sequence
  Synthetic
SDKKVRSRLIECA

SEQ ID NO: 306 - protein, 9 aa, Artificial Sequence
  Synthetic
SKRVAKRKL

SEQ ID NO: 307 - protein, 11 aa, Artificial Sequence
  Synthetic
  misc_feature (2)..(2): Xaa can be any naturally occurring amino acid
  misc_feature (9)..(10): Xaa can be any naturally occurring amino acid
SXGTKRSYXXM

SEQ ID NO: 308 - protein, 14 aa, Artificial Sequence
  Synthetic
TEKKQGKSILYDCA

SEQ ID NO: 309 - protein, 8 aa, Artificial Sequence
  Synthetic
  misc_feature (5)..(7): Xaa can be any naturally occurring amino acid
TKRSXXXM

SEQ ID NO: 310 - protein, 11 aa, Artificial Sequence
  Synthetic
VNEAFETLKRC

SEQ ID NO: 311 - protein, 7 aa, Artificial Sequence
  Synthetic
VSRKRPR

SEQ ID NO: 312 - protein, 13 aa, Artificial Sequence
  Synthetic
YKRPCKRSFIRFI

SEQ ID NO: 313 - protein, 28 aa, Artificial Sequence
  Synthetic
YLTQETNKVETYKEQPLKTPGKKKKGKP

SEQ ID NO: 314 - protein, 15 aa, Artificial Sequence
  Synthetic
YNNQSSNFGPMKGGN

SEQ ID NO: 315 - protein, 33 aa, Artificial Sequence
  Synthetic
KARTPIQKHWRPTVLTEGPPVKIRIETGEWEKA

SEQ ID NO: 316 - protein, 7 aa, Artificial Sequence
  Synthetic
RRMKWKK

SEQ ID NO: 317 - protein, 7 aa, Artificial Sequence
  Synthetic
RRKGKEK

SEQ ID NO: 318 - protein, 8 aa, Artificial Sequence
  Synthetic
RGRRRRQR

SEQ ID NO: 319 - protein, 7 aa, Artificial Sequence
  Synthetic
RIRKKLR

SEQ ID NO: 320 - protein, 17 aa, Artificial Sequence
  Synthetic
RKCLQAGMNLEARKTKK

SEQ ID NO: 321 - protein, 17 aa, Artificial Sequence
  Synthetic
RKEWLTNFMEDRRQRKL

SEQ ID NO: 322 - protein, 20 aa, Artificial Sequence
  Synthetic
  misc_feature (6)..(14): Xaa can be any naturally occurring amino acid
  misc_feature (7)..(14): may not be present
RKKRKXXXXXXXXXKAKKSK

SEQ ID NO: 323 - protein, 9 aa, Artificial Sequence
  Synthetic
RKKRRQRRR

SEQ ID NO: 324 - protein, 19 aa, Artificial Sequence
  Synthetic
RKRAFHGDDPFGEGPPDKK

SEQ ID NO: 325 - protein, 32 aa, Artificial Sequence
  Synthetic
RKRIREDRKATTAQKVQQMKQRLNENERKRKR

SEQ ID NO: 326 - protein, 5 aa, Artificial Sequence
  Synthetic
RKRKK

SEQ ID NO: 327 - protein, 17 aa, Artificial Sequence
  Synthetic
RKRKKMPASQRSKRRKT

SEQ ID NO: 328 - protein, 5 aa, Artificial Sequence
  Synthetic
RKRRR

SEQ ID NO: 329 - protein, 32 aa, Artificial Sequence
  Synthetic
  misc_feature (10)..(28): Xaa can be any naturally occurring amino acid
  misc_feature (11)..(28): may not be present
RLKKLKCSKXXXXXXXXXXXXXXXXXXXKTKR

SEQ ID NO: 330 - protein, 5 aa, Artificial Sequence
  Synthetic
RPRRK

SEQ ID NO: 331 - protein, 12 aa, Artificial Sequence
  Synthetic
RQARRNRRRRWR

SEQ ID NO: 332 - protein, 17 aa, Artificial Sequence
  Synthetic
RRERNKMAAAKCRNRRR

SEQ ID NO: 333 - protein, 15 aa, Artificial Sequence
  Synthetic
  misc_feature (5)..(8): Xaa can be any naturally occurring amino acid
  misc_feature (6)..(8): may not be present
RRERXXXXRPRKIPR

SEQ ID NO: 334 - protein, 38 aa, Artificial Sequence
  Synthetic
  misc_feature (9)..(32): Xaa can be any naturally occurring amino acid
  misc_feature (29)..(31): may not be present
  misc_feature (32)..(35): may not be present
  misc_feature (37)..(37): Xaa can be any naturally occurring amino acid
KRKRKRKRXXXXXXXXXXXXXXXXXXXXXXXXKKKKXK

SEQ ID NO: 335 - protein, 8 aa, Artificial Sequence
  Synthetic
KKKRERLD

SEQ ID NO: 336 - protein, 6 aa, Artificial Sequence
  Synthetic
KRKRRP

SEQ ID NO: 337 - protein, 10 aa, Artificial Sequence
  Synthetic
IKYFKKFPKD

SEQ ID NO: 338 - protein, 19 aa, Artificial Sequence
  Synthetic
  misc_feature (10)..(15): Xaa can be any naturally occurring amino acid
HRKYEAPRHXXXXXXPRKR

SEQ ID NO: 339 - protein, 20 aa, Artificial Sequence
  Synthetic
  misc_feature (4)..(14): Xaa can be any naturally occurring amino acid
KRKXXXXXXXXXXXKKKSKK

SEQ ID NO: 340 - protein, 17 aa, Artificial Sequence
  Synthetic
HRIEEKRKRTYETFKSI

SEQ ID NO: 341 - protein, 34 aa, Artificial Sequence
  Synthetic
  misc_feature (4)..(25): Xaa can be any naturally occurring amino acid
KRKXXXXXXXXXXXXXXXXXXXXXXKELQKQITK

SEQ ID NO: 342 - protein, 17 aa, Artificial Sequence
  Synthetic
KRMRNRIAASKCRKRKL

SEQ ID NO: 343 - protein, 16 aa, Artificial Sequence
  Synthetic
KRPAATKKAGQAKKKK

SEQ ID NO: 344 - protein, 12 aa, Artificial Sequence
  Synthetic
EEDGPQKKKRRL

SEQ ID NO: 345 - protein, 24 aa, Artificial Sequence
  Synthetic
KRPACTLKPECVQQLLVCSQEAKK

SEQ ID NO: 346 - protein, 17 aa, Artificial Sequence
  Synthetic
KRPAEDMEEEQAFKRSR

SEQ ID NO: 347 - protein, 17 aa, Artificial Sequence
  Synthetic
KRPMNAFIVWSRDQRRK

SEQ ID NO: 348 - protein, 17 aa, Artificial Sequence
  Synthetic
KRPMNAFMVWAQAARRK

SEQ ID NO: 349 - protein, 18 aa, Artificial Sequence
  Synthetic
KRKKEMANKSAPEAKKKK

SEQ ID NO: 350 - protein, 11 aa, Artificial Sequence
  Synthetic
APKRKSGVSKC

SEQ ID NO: 351 - protein, 18 aa, Artificial Sequence
  Synthetic
KRAAEDDEDDDVDTKKQK

SEQ ID NO: 352 - protein, 9 aa, Artificial Sequence
  Synthetic
KKKRRSREK

SEQ ID NO: 353 - protein, 14 aa, Artificial Sequence
  Synthetic
KKKKKEEEGEGKKK

SEQ ID NO: 354 - protein, 7 aa, Artificial Sequence
  Synthetic
KKKYKLK

SEQ ID NO: 355 - protein, 17 aa, Artificial Sequence
  Synthetic
KKQTTLAFKPIKKGKKR

SEQ ID NO: 356 - protein, 13 aa, Artificial Sequence
  Synthetic
CKRKTTNADRRKA

SEQ ID NO: 357 - protein, 9 aa, Artificial Sequence
  Synthetic
KKEKKKSKK

SEQ ID NO: 358 - protein, 7 aa, Artificial Sequence
  Synthetic
KHRKHPG

SEQ ID NO: 359 - protein, 15 aa, Artificial Sequence
  Synthetic
  misc_feature (3)..(11): Xaa can be any naturally occurring amino acid
KRXXXXXXXXXKTKK

SEQ ID NO: 360 - protein, 17 aa, Artificial Sequence
  Synthetic
CYGSKNTGAKKRKIDDA

SEQ ID NO: 361 - protein, 17 aa, Artificial Sequence
  Synthetic
KKSKKGRQEALERLKKA

SEQ ID NO: 362 - protein, 20 aa, Artificial Sequence
  Synthetic
KDCVINKHHRNRCQYCRLQR

SEQ ID NO: 363 - protein, 6 aa, Artificial Sequence
  Synthetic
KAKRQR

SEQ ID NO: 364 - protein, 21 aa, Artificial Sequence
  Synthetic
  misc_feature (3)..(17): Xaa can be any naturally occurring amino acid
KKXXXXXXXXXXXXXXXKKRK

SEQ ID NO: 365 - protein, 11 aa, Artificial Sequence
  Synthetic
EYLSRKGKLEL

SEQ ID NO: 366 - protein, 29 aa, Artificial Sequence
  Synthetic
  misc_feature (5)..(24): Xaa can be any naturally occurring amino acid
KRQRXXXXXXXXXXXXXXXXXXXXKKSKK

SEQ ID NO: 367 - protein, 17 aa, Artificial Sequence
  Synthetic
KRSAEGGNPPKPLKKLR

SEQ ID NO: 368 - protein, 8 aa, Artificial Sequence
  Synthetic
APTKRKGS

SEQ ID NO: 369 - protein, 9 aa, Artificial Sequence
  Synthetic
KKKKRKREK

SEQ ID NO: 370 - protein, 5 aa, Artificial Sequence
  Synthetic
KRPRP

SEQ ID NO: 371 - protein, 8 aa, Artificial Sequence
  Synthetic
GRKRKKRT

SEQ ID NO: 372 - protein, 40 aa, Artificial Sequence
  Synthetic
HKKKKIRTSPTFTTPKTLRLRRQPKYPRKSAPRRNKLDHY

SEQ ID NO: 373 - protein, 21 aa, Artificial Sequence
  Synthetic
  misc_feature (4)..(6): Xaa can be any naturally occurring amino acid
  misc_feature (11)..(16): Xaa can be any naturally occurring amino acid
GGGXXXKNRRXXXXXXRGGRN

SEQ ID NO: 374 - protein, 7 aa, Artificial Sequence
  Synthetic
GKKRSKA

SEQ ID NO: 375 - protein, 9 aa, Artificial Sequence
  Synthetic
GKKKYKLKH

SEQ ID NO: 376 - protein, 16 aa, Artificial Sequence
  Synthetic
DEKNIFRRDEKSTMNQ

SEQ ID NO: 377 - protein, 12 aa, Artificial Sequence
  Synthetic
DEKRMQNRMQNR

SEQ ID NO: 378 - protein, 11 aa, Artificial Sequence
  Synthetic
DEKKPLGLKGL

SEQ ID NO: 379 - protein, 26 aa, Artificial Sequence
  Synthetic
  misc_feature (4)..(9): Xaa can be any naturally occurring amino acid
  misc_feature (8)..(9): may not be present
  misc_feature (14)..(14): Xaa can be any naturally occurring amino acid
  misc_feature (17)..(19): Xaa can be any naturally occurring amino acid
  misc_feature (18)..(19): may not be present
RRKXXXXXXRKRKXRKXXXRKRKPLQ

SEQ ID NO: 380 - protein, 17 aa, Artificial Sequence
  Synthetic
  misc_feature (6)..(9): may not be present
RRKRKRKRKPLRKMNQR

SEQ ID NO: 381 - protein, 40 aa, Artificial Sequence
  Synthetic
  misc_feature (6)..(9): may not be present
  misc_feature (10)..(28): Xaa can be any naturally occurring amino acid
  misc_feature (25)..(28): may not be present
  misc_feature (31)..(36): may not be present
RRKRKRKRKXXXXXXXXXXXXXXXXXXXRKRKRKRKQLMK

SEQ ID NO: 382 - protein, 10 aa, Artificial Sequence
  Synthetic
RRKRKRKDEK

SEQ ID NO: 383 - protein, 10 aa, Artificial Sequence
  Synthetic
  misc_feature (4)..(4): Xaa can be any naturally occurring amino acid
DEKXRRKMNQ

SEQ ID NO: 384 - protein, 11 aa, Artificial Sequence
  Synthetic
DERKRRDEPLQ

SEQ ID NO: 385 - protein, 8 aa, Artificial Sequence
  Synthetic
  misc_feature (4)..(4): Xaa can be any naturally occurring amino acid
DERXKKKK

SEQ ID NO: 386 - protein, 17 aa, Artificial Sequence
  Synthetic
  misc_feature (7)..(10): may not be present
DERKRKRKRKGARPLGA

SEQ ID NO: 387 - protein, 14 aa, Artificial Sequence
  Synthetic
  misc_feature (4)..(5): Xaa can be any naturally occurring amino acid
  misc_feature (10)..(11): Xaa can be any naturally occurring amino acid
RPLXXKRKRXXKRV

SEQ ID NO: 388 - protein, 11 aa, Artificial Sequence
  Synthetic
DEKRRRKRFYW

SEQ ID NO: 389 - protein, 10 aa, Artificial Sequence
  Synthetic
RQMPLRRDER

SEQ ID NO: 390 - protein, 8 aa, Artificial Sequence
  Synthetic
RRKKRKKR

SEQ ID NO: 391 - protein, 13 aa, Artificial Sequence
  Synthetic
  misc_feature (4)..(4): Xaa can be any naturally occurring amino acid
  misc_feature (7)..(7): Xaa can be any naturally occurring amino acid
RRKXKRXRKRKDE

SEQ ID NO: 392 - protein, 16 aa, Artificial Sequence
  Synthetic
RSTCMNQRSTCMNQKR

SEQ ID NO: 393 - protein, 11 aa, Artificial Sequence
  Synthetic
  misc_feature (5)..(6): may not be present
  misc_feature (7)..(7): Xaa can be any naturally occurring amino acid
DERRRRXRKPL

SEQ ID NO: 394 - protein, 8 aa, Artificial Sequence
  Synthetic
  misc_feature (2)..(2): Xaa can be any naturally occurring amino acid
RXKKKKDE

SEQ ID NO: 395 - protein, 6 aa, Artificial Sequence
  Synthetic
RRPRRR

SEQ ID NO: 396 - protein, 7 aa, Artificial Sequence
  Synthetic
TPLVKRC

SEQ ID NO: 397 - protein, 10 aa, Artificial Sequence
  Synthetic
  misc_feature (2)..(3): may not be present
  misc_feature (4)..(4): Xaa can be any naturally occurring amino acid
  misc_feature (6)..(7): may not be present
RRRXKKKRST

SEQ ID NO: 398 - protein, 13 aa, Artificial Sequence
  Synthetic
  misc_feature (4)..(4): Xaa can be any naturally occurring amino acid
  misc_feature (4)..(4): may not be present
  misc_feature (11)..(12): may not be present
DKRXQLRKRKRKR

SEQ ID NO: 399 - protein, 12 aa, Artificial Sequence
  Synthetic
  misc_feature (3)..(3): may not be present
  misc_feature (7)..(7): may not be present
RRRKKKKPLRKE

SEQ ID NO: 400 - protein, 10 aa, Artificial Sequence
  Synthetic
  misc_feature (2)..(4): Xaa can be any naturally occurring amino acid
  misc_feature (4)..(4): may not be present
RXXXRRRRRR

SEQ ID NO: 401 - protein, 14 aa, Artificial Sequence
  Synthetic
  misc_feature (2)..(4): Xaa can be any naturally occurring amino acid
  misc_feature (4)..(4): may not be present
  misc_feature (6)..(10): Xaa can be any naturally occurring amino acid
  misc_feature (9)..(10): may not be present
RXXXHXXXXXRRRR

SEQ ID NO: 402 - protein, 9 aa, Artificial Sequence
  Synthetic
DRMNKKKKE

SEQ ID NO: 403 - protein, 8 aa, Artificial Sequence
  Synthetic
WKQKRRKF

SEQ ID NO: 404 - protein, 14 aa, Artificial Sequence
  Synthetic
  misc_feature (2)..(2): Xaa can be any naturally occurring amino acid
  misc_feature (9)..(10): Xaa can be any naturally occurring amino acid
RXKRKRKRXXRKKR

SEQ ID NO: 405 - protein, 12 aa, Artificial Sequence
  Synthetic
  misc_feature (2)..(2): Xaa can be any naturally occurring amino acid
RXKRKRKPLQMR

SEQ ID NO: 406 - protein, 10 aa, Artificial Sequence
  Synthetic
  misc_feature (2)..(2): Xaa can be any naturally occurring amino acid
  misc_feature (7)..(7): Xaa can be any naturally occurring amino acid
RXRRQLXSTR

SEQ ID NO: 407 - protein, 13 aa, Artificial Sequence
  Synthetic
  misc_feature (2)..(2): Xaa can be any naturally occurring amino acid
  misc_feature (4)..(4): Xaa can be any naturally occurring amino acid
  misc_feature (6)..(6): Xaa can be any naturally occurring amino acid
  misc_feature (8)..(8): Xaa can be any naturally occurring amino acid
  misc_feature (10)..(10): Xaa can be any naturally occurring amino acid
  misc_feature (12)..(12): Xaa can be any naturally occurring amino acid
RXRXRXRXRXRXK

SEQ ID NO: 408 - protein, 11 aa, Artificial Sequence
  Synthetic
  misc_feature (2)..(2): Xaa can be any naturally occurring amino acid
  misc_feature (4)..(4): Xaa can be any naturally occurring amino acid
  misc_feature (6)..(6): Xaa can be any naturally occurring amino acid
  misc_feature (8)..(8): Xaa can be any naturally occurring amino acid
  misc_feature (10)..(10): Xaa can be any naturally occurring amino acid
RXRXRXRXRXR

SEQ ID NO: 409 - protein, 10 aa, Artificial Sequence
  Synthetic
  misc_feature (2)..(2): Xaa can be any naturally occurring amino acid
  misc_feature (7)..(7): Xaa can be any naturally occurring amino acid
  misc_feature (7)..(7): may not be present
  misc_feature (9)..(9): Xaa can be any naturally occurring amino acid
RXRSRSXRXR

SEQ ID NO: 410 - protein, 8 aa, Artificial Sequence
  Synthetic
DKQLKKQL

SEQ ID NO: 411 - protein, 13 aa, Artificial Sequence
  Synthetic
  misc_feature (2)..(2): Xaa can be any naturally occurring amino acid
  misc_feature (5)..(10): Xaa can be any naturally occurring amino acid
  misc_feature (9)..(10): may not be present
RXRRXXXXXXRKK

SEQ ID NO: 412 - protein, 8 aa, Artificial Sequence
  Synthetic
RRQMNRRR

SEQ ID NO: 413 - protein, 12 aa, Artificial Sequence
  Synthetic
TSRKKKVLIRPL

SEQ ID NO: 414 - protein, 10 aa, Artificial Sequence
  Synthetic
  misc_feature (3)..(3): Xaa can be any naturally occurring amino acid
  misc_feature (6)..(6): Xaa can be any naturally occurring amino acid
  misc_feature (8)..(8): Xaa can be any naturally occurring amino acid
QLXKRXKXKK

SEQ ID NO: 415 - protein, 28 aa, Artificial Sequence
  Synthetic
  misc_feature (5)..(6): may not be present
  misc_feature (7)..(15): Xaa can be any naturally occurring amino acid
  misc_feature (15)..(18): may not be present
QLKKKKXXXXXXXXXXXXRKQLRKQLKR

SEQ ID NO: 416 - protein, 18 aa, Artificial Sequence
  Synthetic
PVLIRKRKRKRKRKQMNK

SEQ ID NO: 417 - protein, 13 aa, Artificial Sequence
  Synthetic
  misc_feature (3)..(4): Xaa can be any naturally occurring amino acid
PLXXKRIVKPLDE

SEQ ID NO: 418 - protein, 20 aa, Artificial Sequence
  Synthetic
  misc_feature (7)..(8): may not be present
  misc_feature (15)..(15): Xaa can be any naturally occurring amino acid
  misc_feature (19)..(19): Xaa can be any naturally occurring amino acid
PLRKRKRKKPLIRKXPLIXK

SEQ ID NO: 419 - protein, 18 aa, Artificial Sequence
  Synthetic
PLRKRKKRGAPLRKSTQM

SEQ ID NO: 420 - protein, 15 aa, Artificial Sequence
  Synthetic
PLRKRKDEPRRKFYW

SEQ ID NO: 421 - protein, 15 aa, Artificial Sequence
  Synthetic
  misc_feature (5)..(5): Xaa can be any naturally occurring amino acid
PLPLXKRRDEKRQST

SEQ ID NO: 422 - protein, 18 aa, Artificial Sequence
  Synthetic
  misc_feature (13)..(16): may not be present
PLKRKRKRKRKRKRKRPL

SEQ ID NO: 423 - protein, 9 aa, Artificial Sequence
  Synthetic
PLRDEKDER

SEQ ID NO: 424 - protein, 8 aa, Artificial Sequence
  Synthetic
PLRKRKPL

SEQ ID NO: 425 - protein, 16 aa, Artificial Sequence
  Synthetic
  misc_feature (6)..(8): Xaa can be any naturally occurring amino acid
  misc_feature (13)..(14): may not be present
  misc_feature (15)..(15): Xaa can be any naturally occurring amino acid
RKHRKXXXRKRKRKXR

SEQ ID NO: 426 - protein, 15 aa, Artificial Sequence
  Synthetic
  misc_feature (5)..(6): may not be present
  misc_feature (7)..(7): Xaa can be any naturally occurring amino acid
RKKKKKXRKQLRKPL

SEQ ID NO: 427 - protein, 10 aa, Artificial Sequence
  Synthetic
  misc_feature (7)..(7): Xaa can be any naturally occurring amino acid
RKRMSKXKKR

SEQ ID NO: 428 - protein, 16 aa, Artificial Sequence
  Synthetic
  misc_feature (4)..(6): Xaa can be any naturally occurring amino acid
  misc_feature (5)..(6): may not be present
  misc_feature (10)..(11): Xaa can be any naturally occurring amino acid
  misc_feature (11)..(11): may not be present
STGXXXGGGXXGGGST

SEQ ID NO: 429 - protein, 12 aa, Artificial Sequence
  Synthetic
STQMRRRKSTQM

SEQ ID NO: 430 - protein, 12 aa, Artificial Sequence
  Synthetic
STQMRKRRSTQM

SEQ ID NO: 431 - protein, 12 aa, Artificial Sequence
  Synthetic
STQMRKRKSTQM

SEQ ID NO: 432 - protein, 23 aa, Artificial Sequence
  Synthetic
  misc_feature (16)..(19): Xaa can be any naturally occurring amino acid
  misc_feature (19)..(19): may not be present
RKRKRKRKQMNPLRKXXXXRKRK

SEQ ID NO: 433 - protein, 30 aa, Artificial Sequence
  Synthetic
  misc_feature (7)..(22): Xaa can be any naturally occurring amino acid
  misc_feature (15)..(22): may not be present
RKRKRKXXXXXXXXXXXXXXXXRKRKRKRK

SEQ ID NO: 434 - protein, 27 aa, Artificial Sequence
  Synthetic
  misc_feature (7)..(7): Xaa can be any naturally occurring amino acid
  misc_feature (10)..(10): Xaa can be any naturally occurring amino acid
  misc_feature (13)..(21): Xaa can be any naturally occurring amino acid
  misc_feature (17)..(21): may not be present
RKRKRKXRKXRKXXXXXXXXXRKRKRK

SEQ ID NO: 435 - protein, 25 aa, Artificial Sequence
  Synthetic
  misc_feature (5)..(8): may not be present
  misc_feature (9)..(12): Xaa can be any naturally occurring amino acid
  misc_feature (11)..(12): may not be present
  misc_feature (20)..(21): may not be present
RKRKRKRKXXXXQLMRKRKRKRKKR

SEQ ID NO: 436 - protein, 45 aa, Artificial Sequence
  Synthetic
  misc_feature (5)..(8): may not be present
  misc_feature (9)..(10): Xaa can be any naturally occurring amino acid
  misc_feature (10)..(10): may not be present
  misc_feature (11)..(14): may not be present
  misc_feature (21)..(24): may not be present
  misc_feature (25)..(29): Xaa can be any naturally occurring amino acid
  misc_feature (28)..(29): may not be present
  misc_feature (32)..(33): Xaa can be any naturally occurring amino acid
  misc_feature (32)..(33): may not be present
  misc_feature (40)..(43): may not be present
RKRKRKRKXXRKRKRKRKRKRKRKXXXXXRKXXRKRKRKRKRKPL

SEQ ID NO: 437 - protein, 17 aa, Artificial Sequence
  Synthetic
  misc_feature (3)..(3): Xaa can be any naturally occurring amino acid
  misc_feature (6)..(6): Xaa can be any naturally occurring amino acid
  misc_feature (9)..(14): Xaa can be any naturally occurring amino acid
  misc_feature (13)..(14): may not be present
RKXRKXKRXXXXXXRKK

SEQ ID NO: 438 - protein, 21 aa, Artificial Sequence
  Synthetic
  misc_feature (13)..(16): may not be present
RKPLIVKRRKRKRKRKPLVIR

SEQ ID NO: 439 - protein, 8 aa, Artificial Sequence
  Synthetic
  misc_feature (4)..(5): Xaa can be any naturally occurring amino acid
PLKXXKRR

SEQ ID NO: 440 - protein, 11 aa, Artificial Sequence
  Synthetic
PLVRKSTRDEK

SEQ ID NO: 441 - protein, 9 aa, Artificial Sequence
  Synthetic
YFWRRRRPL

SEQ ID NO: 442 - protein, 18 aa, Artificial Sequence
  Synthetic
  misc_feature (9)..(10): Xaa can be any naturally occurring amino acid
KRDEKRDEXXKRKRKRKR

SEQ ID NO: 443 - protein, 18 aa, Artificial Sequence
  Synthetic
  misc_feature (3)..(4): Xaa can be any naturally occurring amino acid
  misc_feature (8)..(15): Xaa can be any naturally occurring amino acid
  misc_feature (14)..(15): may not be present
KRXXKNKXXXXXXXXKKR

SEQ ID NO: 444 - protein, 6 aa, Artificial Sequence
  Synthetic
KRKRKK

SEQ ID NO: 445 - protein, 11 aa, Artificial Sequence
  Synthetic
  misc_feature (5)..(6): Xaa can be any naturally occurring amino acid
KRGGXXGGGRK

SEQ ID NO: 446 - protein, 13 aa, Artificial Sequence
  Synthetic
  misc_feature (7)..(7): Xaa can be any naturally occurring amino acid
  misc_feature (9)..(9): Xaa can be any naturally occurring amino acid
QMNRRKXKXRKRK

SEQ ID NO: 447 - protein, 11 aa, Artificial Sequence
  Synthetic
  misc_feature (7)..(7): Xaa can be any naturally occurring amino acid
GAKRKRXKRGA

SEQ ID NO: 448 - protein, 14 aa, Artificial Sequence
  Synthetic
  misc_feature (4)..(4): Xaa can be any naturally occurring amino acid
  misc_feature (7)..(7): Xaa can be any naturally occurring amino acid
  misc_feature (12)..(12): Xaa can be any naturally occurring amino acid
GARXRKXRKRKXQM

SEQ ID NO: 449 - protein, 10 aa, Artificial Sequence
  Synthetic
  misc_feature (4)..(4): Xaa can be any naturally occurring amino acid
GAKXKKKMNQ

SEQ ID NO: 450 - protein, 11 aa, Artificial Sequence
  Synthetic
GAPLVRKRKKR

SEQ ID NO: 451 - protein, 8 aa, Artificial Sequence
  Synthetic
EDRRRRED

SEQ ID NO: 452 - protein, 11 aa, Artificial Sequence
  Synthetic
DESTPLKRSTC

SEQ ID NO: 453 - protein, 14 aa, Artificial Sequence
  Synthetic
KRKRKRKRKRKRKR

SEQ ID NO: 454 - protein, 14 aa, Artificial Sequence
  Synthetic
KRKRQMNRRKQMNR

SEQ ID NO: 455 - protein, 16 aa, Artificial Sequence
  Synthetic
  misc_feature (5)..(5): Xaa can be any naturally occurring amino acid
  misc_feature (12)..(12): Xaa can be any naturally occurring amino acid
KRKRXKRKRKRXKRKR

SEQ ID NO: 456 - protein, 15 aa, Artificial Sequence
  Synthetic
  misc_feature (7)..(7): Xaa can be any naturally occurring amino acid
PLVKRKXRKRKRKPL

SEQ ID NO: 457 - protein, 13 aa, Artificial Sequence
  Synthetic
  misc_feature (7)..(7): Xaa can be any naturally occurring amino acid
PLVKRKXQMNRKR

SEQ ID NO: 458 - protein, 13 aa, Artificial Sequence
  Synthetic
  misc_feature (6)..(9): Xaa can be any naturally occurring amino acid
  misc_feature (9)..(9): may not be present
PLQKRXXXXKKRK

SEQ ID NO: 459 - protein, 30 aa, Artificial Sequence
  Synthetic
  misc_feature (7)..(8): Xaa can be any naturally occurring amino acid
  misc_feature (8)..(8): may not be present
  misc_feature (11)..(16): Xaa can be any naturally occurring amino acid
  misc_feature (14)..(16): may not be present
  misc_feature (21)..(22): Xaa can be any naturally occurring amino acid
  misc_feature (22)..(22): may not be present
  misc_feature (25)..(26): Xaa can be any naturally occurring amino acid
  misc_feature (26)..(26): may not be present
PLQKRKXXRKXXXXXXRKRKXXRKXXRKRK

SEQ ID NO: 460 - protein, 22 aa, Artificial Sequence
  Synthetic
  misc_feature (14)..(14): Xaa can be any naturally occurring amino acid
PLQMNKRKKRKRRXKPLQMNKR

SEQ ID NO: 461 - protein, 16 aa, Artificial Sequence
  Synthetic
  misc_feature (15)..(15): Xaa can be any naturally occurring amino acid
PLQMKRRKRQMKRRXK

SEQ ID NO: 462 - protein, 13 aa, Artificial Sequence
  Synthetic
MIVWSRDHEQRRK

SEQ ID NO: 463 - protein, 20 aa, Artificial Sequence
  Synthetic
LFSTKVIQMKRRQMVISTKL

SEQ ID NO: 464 - protein, 54 aa, Artificial Sequence
  Synthetic
  misc_feature (5)..(5): Xaa can be any naturally occurring amino acid
  misc_feature (5)..(5): may not be present
  misc_feature (10)..(13): may not be present
  misc_feature (14)..(47): Xaa can be any naturally occurring amino acid
  misc_feature (39)..(47): may not be present
  misc_feature (50)..(51): may not be present
  misc_feature (52)..(53): Xaa can be any naturally occurring amino acid
  misc_feature (53)..(53): may not be present
KRKRXKRKRKRKRXXXXXXXXXXXXXXXXXXXXXXXXXXXXXXXXXXKKKKXXK

SEQ ID NO: 465 - protein, 22 aa, Artificial Sequence
  Synthetic
  misc_feature (7)..(10): Xaa can be any naturally occurring amino acid
  misc_feature (8)..(10): may not be present
  misc_feature (15)..(19): Xaa can be any naturally occurring amino acid
  misc_feature (16)..(19): may not be present
KRKRPLXXXXKRKRXXXXXKKK

SEQ ID NO: 466 - protein, 15 aa, Artificial Sequence
  Synthetic
  misc_feature (5)..(6): may not be present
  misc_feature (7)..(8): Xaa can be any naturally occurring amino acid
KRKRKRXXKRKRQLM

SEQ ID NO: 467 - protein, 15 aa, Artificial Sequence
  Synthetic
  misc_feature (9)..(9): Xaa can be any naturally occurring amino acid
DERKRKRKXKRKRPL

SEQ ID NO: 468 - protein, 6 aa, Artificial Sequence
  Synthetic
KKRKRT

SEQ ID NO: 469 - protein, 27 aa, Artificial Sequence
  Synthetic
  misc_feature (4)..(17): Xaa can be any naturally occurring amino acid
  misc_feature (14)..(17): may not be present
  misc_feature (24)..(24): Xaa can be any naturally occurring amino acid
KRKXXXXXXXXXXXXXXKRKRKRXKRK

SEQ ID NO: 470 - protein, 15 aa, Artificial Sequence
  Synthetic
PKKRKRPRAKKTVSE

SEQ ID NO: 471 - protein, 22 aa, Artificial Sequence
  Synthetic
  misc_feature (5)..(12): Xaa can be any naturally occurring amino acid
  misc_feature (10)..(12): may not be present
  misc_feature (14)..(17): Xaa can be any naturally occurring amino acid
  misc_feature (16)..(17): may not be present
  misc_feature (19)..(19): Xaa can be any naturally occurring amino acid
  misc_feature (21)..(21): Xaa can be any naturally occurring amino acid
PKRPXXXXXXXXLXXXXRXKXK

SEQ ID NO: 472 - protein, 11 aa, Artificial Sequence
  Synthetic
  misc_feature (4)..(7): Xaa can be any naturally occurring amino acid
  misc_feature (6)..(7): may not be present
KRKXXXXDRRK

SEQ ID NO: 473 - protein, 7 aa, Artificial Sequence
  Synthetic
  misc_feature (5)..(5): Xaa can be any naturally occurring amino acid
PKKKXRK

SEQ ID NO: 474 - protein, 18 aa, Artificial Sequence
  Synthetic
  misc_feature (4)..(13): Xaa can be any naturally occurring amino acid
  misc_feature (9)..(13): may not be present
KRKXXXXXXXXXXKKPLK

SEQ ID NO: 475 - protein, 15 aa, Artificial Sequence
  Synthetic
NQRRQRKEGKRIVLS

SEQ ID NO: 476 - protein, 6 aa, Artificial Sequence
  Synthetic
LKKIKQ

SEQ ID NO: 477 - protein, 15 aa, Artificial Sequence
  Synthetic
KRGPLRGPLRGLPRK

SEQ ID NO: 478 - protein, 12 aa, Artificial Sequence
  Synthetic
KRMNSQRMNSQR

SEQ ID NO: 479 - protein, 16 aa, Artificial Sequence
  Synthetic
  misc_feature (4)..(11): Xaa can be any naturally occurring amino acid
  misc_feature (4)..(11): may not be present
KRKXXXXXXXXKRPLK

SEQ ID NO: 480 - protein, 9 aa, Artificial Sequence
  Synthetic
KRGRGRPRK

SEQ ID NO: 481 - protein, 18 aa, Artificial Sequence
  Synthetic
  misc_feature (3)..(9): Xaa can be any naturally occurring amino acid
  misc_feature (4)..(9): may not be present
KKXXXXXXXKPLPLIVKK

SEQ ID NO: 482 - protein, 21 aa, Artificial Sequence
  Synthetic
  misc_feature (8)..(15): Xaa can be any naturally occurring amino acid
  misc_feature (12)..(15): may not be present
RERMNQKXXXXXXXXRMNQRR

SEQ ID NO: 483 - protein, 7 aa, Artificial Sequence
  Synthetic
KKRKRKR

SEQ ID NO: 484 - protein, 7 aa, Artificial Sequence
  Synthetic
KKRKRST

SEQ ID NO: 485 - protein, 11 aa, Artificial Sequence
  Synthetic
  misc_feature (9)..(9): Xaa can be any naturally occurring amino acid
QRKHRKRKXRR

SEQ ID NO: 486 - protein, 5 aa, Artificial Sequence
  Synthetic
KKRRK

SEQ ID NO: 487 - protein, 14 aa, Artificial Sequence
  Synthetic
  misc_feature (5)..(5): Xaa can be any naturally occurring amino acid
  misc_feature (7)..(7): Xaa can be any naturally occurring amino acid
QNRRXKXRKRKDQE

SEQ ID NO: 488 - protein, 17 aa, Artificial Sequence
  Synthetic
  misc_feature (2)..(2): Xaa can be any naturally occurring amino acid
  misc_feature (13)..(14): may not be present
  misc_feature (15)..(15): Xaa can be any naturally occurring amino acid
PXPQLVMNKRKRKRXKQ

SEQ ID NO: 489 - protein, 6 aa, Artificial Sequence
  Synthetic
  misc_feature (5)..(5): Xaa can be any naturally occurring amino acid
KKRRXK

SEQ ID NO: 490 - protein, 22 aa, Artificial Sequence
  Synthetic
KKMNQSTCRMNQSTCKMNQSTC

SEQ ID NO: 491 - protein, 14 aa, Artificial Sequence
  Synthetic
  misc_feature (3)..(3): Xaa can be any naturally occurring amino acid
  misc_feature (5)..(9): Xaa can be any naturally occurring amino acid
  misc_feature (8)..(9): may not be present
KKXRXXXXXRPVLK

SEQ ID NO: 492 - protein, 7 aa, Artificial Sequence
  Synthetic
  misc_feature (4)..(4): may not be present
  misc_feature (7)..(7): may not be present
KKKKRRR

SEQ ID NO: 493 - protein, 7 aa, Artificial Sequence
  Synthetic
KRRQMRR

SEQ ID NO: 494 - protein, 16 aa, Artificial Sequence
  Synthetic
  misc_feature (2)..(3): Xaa can be any naturally occurring amino acid
  misc_feature (5)..(5): Xaa can be any naturally occurring amino acid
  misc_feature (7)..(7): Xaa can be any naturally occurring amino acid
  misc_feature (9)..(13): Xaa can be any naturally occurring amino acid
KXXKXKXKXXXXXRKK

SEQ ID NO: 495 - protein, 10 aa, Artificial Sequence
  Synthetic
KGAKAGKKAG

SEQ ID NO: 496 - protein, 33 aa, Artificial Sequence
  Synthetic
  misc_feature (7)..(10): Xaa can be any naturally occurring amino acid
  misc_feature (9)..(10): may not be present
  misc_feature (13)..(14): may not be present
  misc_feature (13)..(14): Xaa can be any naturally occurring amino acid
  misc_feature (16)..(20): Xaa can be any naturally occurring amino acid
  misc_feature (19)..(20): may not be present
  misc_feature (23)..(24): Xaa can be any naturally occurring amino acid
  misc_feature (23)..(24): may not be present
  misc_feature (27)..(28): Xaa can be any naturally occurring amino acid
  misc_feature (27)..(28): may not be present
KRRKRKXXXXRKXXRXXXXXRKXXRKXXRKRKK

SEQ ID NO: 497 - protein, 14 aa, Artificial Sequence
  Synthetic
  misc_feature (6)..(7): Xaa can be any naturally occurring amino acid
  misc_feature (10)..(11): may not be present
KRSTRXXRRRRQLK

SEQ ID NO: 498 - protein, 16 aa, Artificial Sequence
  Synthetic
  misc_feature (3)..(3): Xaa can be any naturally occurring amino acid
  misc_feature (5)..(5): Xaa can be any naturally occurring amino acid
  misc_feature (7)..(12): Xaa can be any naturally occurring amino acid
  misc_feature (9)..(12): may not be present
KRXRXRXXXXXXRKRK

SEQ ID NO: 499 - protein, 10 aa, Artificial Sequence
  Synthetic
  misc_feature (3)..(3): Xaa can be any naturally occurring amino acid
  misc_feature (5)..(6): Xaa can be any naturally occurring amino acid
KRXRXXRRLK

SEQ ID NO: 500 - protein, 7 aa, Artificial Sequence
  Synthetic
KRRRLVI

SEQ ID NO: 501 - protein, 24 aa, Artificial Sequence
  Synthetic
  misc_feature (4)..(5): may not be present
  misc_feature (6)..(11): Xaa can be any naturally occurring amino acid
  misc_feature (9)..(11): may not be present
  misc_feature (16)..(19): may not be present
  misc_feature (20)..(23): Xaa can be any naturally occurring amino acid
  misc_feature (20)..(22): may not be present
KRRRRXXXXXXRKRKRKRKXXXKR

SEQ ID NO: 502 - protein, 7 aa, Artificial Sequence
  Synthetic
KKRRDEK

SEQ ID NO: 503 - protein, 11 aa, Artificial Sequence
  Synthetic
  misc_feature (3)..(3): Xaa can be any naturally occurring amino acid
  misc_feature (10)..(10): Xaa can be any naturally occurring amino acid
KRXDEKRKRXK

SEQ ID NO: 504 - protein, 10 aa, Artificial Sequence
  Synthetic
  misc_feature (3)..(4): Xaa can be any naturally occurring amino acid
  misc_feature (7)..(7): Xaa can be any naturally occurring amino acid
KRXXKKXKDE

SEQ ID NO: 505 - protein, 13 aa, Artificial Sequence
  Synthetic
KIVQMRRVISTKL

SEQ ID NO: 506 - protein, 9 aa, Artificial Sequence
  Synthetic
KKRKRRRKR

SEQ ID NO: 507 - protein, 13 aa, Artificial Sequence
  Synthetic
KKRQMNRKRQMNR

SEQ ID NO: 508 - protein, 11 aa, Artificial Sequence
  Synthetic
  misc_feature (2)..(2): Xaa can be any naturally occurring amino acid
KXPLVRKRKRK

SEQ ID NO: 509 - protein, 13 aa, Artificial Sequence
  Synthetic
  misc_feature (2)..(2): Xaa can be any naturally occurring amino acid
  misc_feature (4)..(4): Xaa can be any naturally occurring amino acid
  misc_feature (6)..(10): Xaa can be any naturally occurring amino acid
KXKXKXXXXXRKK

SEQ ID NO: 510 - protein, 6 aa, Artificial Sequence
  Synthetic
  misc_feature (2)..(2): Xaa can be any naturally occurring amino acid
KXKRQR

SEQ ID NO: 511 - protein, 12 aa, Artificial Sequence
  Synthetic
KRPLVGAKRKPL

SEQ ID NO: 512 - protein, 18 aa, Artificial Sequence
  Synthetic
  misc_feature (8)..(15): Xaa can be any naturally occurring amino acid
  misc_feature (11)..(15): may not be present
KRKRKQLXXXXXXXXRRR

SEQ ID NO: 513 - protein, 12 aa, Artificial Sequence
  Synthetic
  misc_feature (6)..(9): may not be present
KRKRKRKRKSTH

SEQ ID NO: 514 - protein, 9 aa, Artificial Sequence
  Synthetic
  misc_feature (7)..(7): Xaa can be any naturally occurring amino acid
KPLKKKXKK

SEQ ID NO: 515 - protein, 29 aa, Artificial Sequence
  Synthetic
  misc_feature (6)..(6): may not be present
  misc_feature (7)..(9): Xaa can be any naturally occurring amino acid
  misc_feature (8)..(9): may not be present
  misc_feature (14)..(17): may not be present
  misc_feature (18)..(26): Xaa can be any naturally occurring amino acid
  misc_feature (24)..(26): may not be present
KPLKKKXXXRKRKRKRKXXXXXXXXXKKR

SEQ ID NO: 516 - protein, 11 aa, Artificial Sequence
  Synthetic
KPLMNRRKMNQ

SEQ ID NO: 517 - protein, 13 aa, Artificial Sequence
  Synthetic
KMNQRRPLVIKPL

SEQ ID NO: 518 - protein, 16 aa, Artificial Sequence
  Synthetic
  misc_feature (3)..(5): Xaa can be any naturally occurring amino acid
  misc_feature (4)..(5): may not be present
  misc_feature (7)..(11): Xaa can be any naturally occurring amino acid
  misc_feature (10)..(11): may not be present
KRXXXHXXXXXRLQRR

SEQ ID NO: 519 - protein, 12 aa, Artificial Sequence
  Synthetic
  misc_feature (4)..(4): Xaa can be any naturally occurring amino acid
  misc_feature (6)..(6): Xaa can be any naturally occurring amino acid
  misc_feature (11)..(11): Xaa can be any naturally occurring amino acid
RPLXGXKRKRXK

SEQ ID NO: 520 - protein, 9 aa, Artificial Sequence
  Synthetic
  misc_feature (3)..(3): Xaa can be any naturally occurring amino acid
  misc_feature (5)..(5): Xaa can be any naturally occurring amino acid
  misc_feature (7)..(7): Xaa can be any naturally occurring amino acid
RRXRXKXKQ

SEQ ID NO: 521 - protein, 9 aa, Artificial Sequence
  Synthetic
  misc_feature (3)..(3): Xaa can be any naturally occurring amino acid
RRXRPVLRK

SEQ ID NO: 522 - protein, 8 aa, Artificial Sequence
  Synthetic
  misc_feature (3)..(3): Xaa can be any naturally occurring amino acid
RRXRRRRR

SEQ ID NO: 523 - protein, 10 aa, Artificial Sequence
  Synthetic
  misc_feature (3)..(3): Xaa can be any naturally occurring amino acid
  misc_feature (6)..(6): Xaa can be any naturally occurring amino acid
RRXKRXKPLV

SEQ ID NO: 524 - protein, 11 aa, Artificial Sequence
  Synthetic
  misc_feature (5)..(5): Xaa can be any naturally occurring amino acid
RRTSXQKKRKN

SEQ ID NO: 525 - protein, 12 aa, Artificial Sequence
  Synthetic
  misc_feature (5)..(5): Xaa can be any naturally occurring amino acid
RRTSXQKKRKNS

SEQ ID NO: 526 - protein, 12 aa, Artificial Sequence
  Synthetic
  misc_feature (8)..(8): Xaa can be any naturally occurring amino acid
RRPLQMNXRRRR

SEQ ID NO: 527 - protein, 10 aa, Artificial Sequence
  Synthetic
  misc_feature (9)..(9): Xaa can be any naturally occurring amino acid
RRPLIVRKXK

SEQ ID NO: 528 - protein, 12 aa, Artificial Sequence
  Synthetic
  misc_feature (3)..(4): may not be present
  misc_feature (7)..(9): Xaa can be any naturally occurring amino acid
  misc_feature (8)..(9): may not be present
GGGGRKXXXGGG

SEQ ID NO: 529 - protein, 8 aa, Artificial Sequence
  Synthetic
  misc_feature (6)..(7): may not be present
RRRRRRRT

SEQ ID NO: 530 - protein, 8 aa, Artificial Sequence
  Synthetic
  misc_feature (3)..(3): Xaa can be any naturally occurring amino acid
  misc_feature (5)..(5): Xaa can be any naturally occurring amino acid
RRXRXRKQ

SEQ ID NO: 531 - protein, 7 aa, Artificial Sequence
  Synthetic
  misc_feature (3)..(4): Xaa can be any naturally occurring amino acid
RRXXKRK

SEQ ID NO: 532 - protein, 8 aa, Artificial Sequence
  Synthetic
  misc_feature (3)..(3): may not be present
  misc_feature (3)..(3): Xaa can be any naturally occurring amino acid
RRXRRRRR

SEQ ID NO: 533 - protein, 18 aa, Artificial Sequence
  Synthetic
  misc_feature (5)..(12): Xaa can be any naturally occurring amino acid
  misc_feature (9)..(12): may not be present
RMNQXXXXXXXXRMNQRR

SEQ ID NO: 534 - protein, 10 aa, Artificial Sequence
  Synthetic
  misc_feature (9)..(9): Xaa can be any naturally occurring amino acid
RMNQRRRRXR

SEQ ID NO: 535 - protein, 12 aa, Artificial Sequence
  Synthetic
  misc_feature (8)..(9): may not be present
RKRKRKRKRKDE

SEQ ID NO: 536 - protein, 34 aa, Artificial Sequence
  Synthetic
  misc_feature (6)..(7): Xaa can be any naturally occurring amino acid
  misc_feature (6)..(7): may not be present
  misc_feature (10)..(11): Xaa can be any naturally occurring amino acid
  misc_feature (10)..(11): may not be present
  misc_feature (14)..(18): Xaa can be any naturally occurring amino acid
  misc_feature (17)..(18): may not be present
  misc_feature (21)..(22): may not be present
  misc_feature (21)..(22): Xaa can be any naturally occurring amino acid
RKRRKXXRKXXRKXXXXXRKXXRKRKRKRKPMQL

SEQ ID NO: 537 - protein, 9 aa, Artificial Sequence
  Synthetic
  misc_feature (8)..(8): Xaa can be any naturally occurring amino acid
RKRRRRRXR

SEQ ID NO: 538 - protein, 12 aa, Artificial Sequence
  Synthetic
RIVLPIVLPKRR

SEQ ID NO: 539 - protein, 12 aa, Artificial Sequence
  Synthetic
  misc_feature (11)..(11): Xaa can be any naturally occurring amino acid
RGVLIPRRRRXR

SEQ ID NO: 540 - protein, 16 aa, Artificial Sequence
  Synthetic
  misc_feature (4)..(5): Xaa can be any naturally occurring amino acid
  misc_feature (4)..(5): may not be present
  misc_feature (11)..(11): Xaa can be any naturally occurring amino acid
RGAXXGARGAXGARGA

SEQ ID NO: 541 - protein, 10 aa, Artificial Sequence
  Synthetic
RGAIVLPKRR

SEQ ID NO: 542 - protein, 16 aa, Artificial Sequence
  Synthetic
  misc_feature (7)..(10): may not be present
  misc_feature (11)..(12): Xaa can be any naturally occurring amino acid
GRRKRKRKRKXXRKQL

SEQ ID NO: 543 - protein, 7 aa, Artificial Sequence
  Synthetic
RRRPLRK

SEQ ID NO: 544 - protein, 11 aa, Artificial Sequence
  Synthetic
  misc_feature (6)..(7): Xaa can be any naturally occurring amino acid
RRRLPXXRPLQ

SEQ ID NO: 545 - protein, 8 aa, Artificial Sequence
  Synthetic
  misc_feature (6)..(6): Xaa can be any naturally occurring amino acid
RRRRRXRR

SEQ ID NO: 546 - protein, 9 aa, Artificial Sequence
  Synthetic
  misc_feature (6)..(7): may not be present
RKRRRRRST

SEQ ID NO: 547 - protein, 23 aa, Artificial Sequence
  Synthetic
  misc_feature (4)..(19): Xaa can be any naturally occurring amino acid
  misc_feature (16)..(19): may not be present
RKRXXXXXXXXXXXXXXXXRRKK

SEQ ID NO: 548 - protein, 15 aa, Artificial Sequence
  Synthetic
RKRPLQMNRPLQMNR

SEQ ID NO: 549 - protein, 7 aa, Artificial Sequence
  Synthetic
RKRKRKR

SEQ ID NO: 550 - protein, 13 aa, Artificial Sequence
  Synthetic
  misc_feature (6)..(11): Xaa can be any naturally occurring amino acid
  misc_feature (9)..(11): may not be present
KKKKKXXXXXXKK

SEQ ID NO: 551 - protein, 7 aa, Artificial Sequence
  Synthetic
KKKKRKR

SEQ ID NO: 552 - protein, 7 aa, Artificial Sequence
  Synthetic
  misc_feature (6)..(6): Xaa can be any naturally occurring amino acid
RKKRRXR

SEQ ID NO: 553 - protein, 9 aa, Artificial Sequence
  Synthetic
KKKRKRVPL

SEQ ID NO: 554 - protein, 6 aa, Artificial Sequence
  Synthetic
RKKRKR

SEQ ID NO: 555 - protein, 18 aa, Artificial Sequence
  Synthetic
  misc_feature (4)..(12): Xaa can be any naturally occurring amino acid
  misc_feature (10)..(12): may not be present
  misc_feature (14)..(16): Xaa can be any naturally occurring amino acid
  misc_feature (15)..(16): may not be present
KKPXXXXXXXXXKXXXRK

SEQ ID NO: 556 - protein, 14 aa, Artificial Sequence
  Synthetic
RKIVEWMLTQRNHF

SEQ ID NO: 557 - protein, 12 aa, Artificial Sequence
  Synthetic
RKPLPLVKKRKH

SEQ ID NO: 558 - protein, 11 aa, Artificial Sequence
  Synthetic
  misc_feature (10)..(10): Xaa can be any naturally occurring amino acid
RKRKQMLRKXR

SEQ ID NO: 559 - protein, 9 aa, Artificial Sequence
  Synthetic
  misc_feature (7)..(8): may not be present
RRRRRRXXR

SEQ ID NO: 560 - protein, 6 aa, Artificial Sequence
  Synthetic
RRRRRR

SEQ ID NO: 561 - protein, 8 aa, Artificial Sequence
  Synthetic
RRRKSTCK

SEQ ID NO: 562 - protein, 6 aa, Artificial Sequence
  Synthetic
RRRKKR

SEQ ID NO: 563 - protein, 13 aa, Artificial Sequence
  Synthetic
  misc_feature (4)..(10): Xaa can be any naturally occurring amino acid
  misc_feature (9)..(10): may not be present
RRKXXXXXXXRRR

SEQ ID NO: 564 - protein, 17 aa, Artificial Sequence
  Synthetic
  misc_feature (4)..(8): Xaa can be any naturally occurring amino acid
  misc_feature (7)..(8): may not be present
RRKXXXXXRDERRRPLV

SEQ ID NO: 565 - protein, 23 aa, Artificial Sequence
  Synthetic
  misc_feature (9)..(16): Xaa can be any naturally occurring amino acid
  misc_feature (13)..(16): may not be present
RRERMNQKXXXXXXXXRMNQRRR

SEQ ID NO: 566 - protein, 6 aa, Artificial Sequence
  Synthetic
KHLKGR

SEQ ID NO: 567 - protein, 23 aa, Artificial Sequence
  Synthetic
  misc_feature (3)..(14): Xaa can be any naturally occurring amino acid
  misc_feature (10)..(14): may not be present
RKXXXXXXXXXXXXRKSTMNQKK

SEQ ID NO: 568 - protein, 29 aa, Artificial Sequence
  Synthetic
  misc_feature (5)..(8): may not be present
  misc_feature (13)..(23): Xaa can be any naturally occurring amino acid
  misc_feature (20)..(23): may not be present
RKRKRKRKPLRKXXXXXXXXXXXRKQLKH

SEQ ID NO: 569 - protein, 20 aa, Artificial Sequence
  Synthetic
  misc_feature (6)..(9): Xaa can be any naturally occurring amino acid
  misc_feature (8)..(9): may not be present
  misc_feature (14)..(17): may not be present
RHRKHXXXXRKRKRKRKPLR

SEQ ID NO: 570 - protein, 8 aa, Artificial Sequence
  Synthetic
PKKKRAVE